(19) United States
(12) Patent Application Publication
(10) Pub. No.: US 2022/0290160 A1
(43) Pub. Date: Sep. 15, 2022

(54) COMPOSITIONS AND METHODS FOR CLL1 MODIFICATION

(71) Applicant: VOR BIOPHARMA INC., Cambridge, MA (US)

(72) Inventors: John Lydeard, Cambridge, MA (US); Chong Luo, Boston, MA (US); Michelle Lin, Arlington, MA (US)

(73) Assignee: VOR BIOPHARMA INC., Cambridge, MA (US)

(21) Appl. No.: 17/638,607

(22) PCT Filed: Aug. 28, 2020
(86) PCT No.: PCT/US2020/048617

§ 371 Date: Feb. 25, 2022

Related U.S. Application Data
(60) Provisional application No. 62/962,133, filed on Jan. 16, 2020.
(60) Provisional application No. 62/892,868, filed on Aug. 28, 2019.

(51) Int. Cl. C12N 15/113

(52) U.S. Cl. CPC C12N 15/1138; C12N 2310/20; C12N 2310/315; C12N 2310/346; C12N 2320/11; C12N 2320/31

(57) ABSTRACT

This disclosure provides, e.g., novel cells having a modification (e.g., insertion or deletion) in the endogenous CLL1 gene. The disclosure also provides compositions, e.g., gRNAs, that can be used to make such a modification.

### RELATED APPLICATIONS

This application claims priority to U.S. Ser. No. 62/892,868 filed Aug. 28, 2019 and U.S. Ser. No. 62/962,133 filed Jan. 16, 2020, the entire contents of each of which are incorporated herein by reference.

### SEQUENCE LISTING

The instant application contains a Sequence Listing which has been submitted electronically in ASCII format and is hereby incorporated by reference in its entirety. Said ASCII copy, created on Aug. 28, 2020, is named V0291_70005WO00_SL.txt and is 54,483 bytes in size.

### BACKGROUND

When a cancer patient is administered an anti-CLL1 cancer therapy, the therapy can deplete not only CLL1+ cancer cells, but also noncancerous CLL1+ cells in an “on-target, off-tumor” effect. Since certain hematopoietic cells typically express CLL1, the loss of the noncancerous CLL1+ cells can deplete the hematopoietic system of the patient. To address this depletion, the subject can be administered rescue cells (e.g., HSCs and/or HPCs) comprising a modification in the CLL1 gene. These CLL1-modified cells can be resistant to the anti-CLL1 cancer therapy, and can therefore repopulate the hematopoietic system during or after anti-CLL1 therapy.

### SUMMARY OF THE INVENTION

Some aspects of this disclosure provide, e.g., novel cells having a modification (e.g., substitution, insertion or deletion) in the endogenous CLL1 gene. Some aspects of this disclosure also provide compositions, e.g., gRNAs, that can be used to make such a modification. Some aspects of this disclosure provide methods of using the compositions provided herein, e.g., methods of using certain gRNAs provided to create genetically engineered cells, e.g., cells having a modification in the endogenous CLL1 gene. Some aspects of this disclosure provide methods of administering genetically engineered cells provided herein, e.g., cells having a modification in the endogenous CLL1 gene, to a subject in need thereof. Some aspects of this disclosure provide strategies, compositions, methods, and treatment modalities for the treatment of patients having cancer and receiving or in need of receiving an anti-CLL1 cancer therapy.

### ENUMERATED EMBODIMENTS

1. A gRNA comprising a targeting domain which binds a target domain of Table 1 (e.g., a target domain of any of SEQ ID NOS: 1-20 or 40-43).
2. A gRNA comprising a targeting domain capable of directing cleavage or editing of a target domain of Table 1 (e.g., a target domain of any of SEQ ID NOS: 1-20 or 40-43).
3. A gRNA comprising a targeting domain which binds a target domain of any of SEQ ID NOS: 1-3 or 5-10, or SEQ ID NOS: 11-13 or 15-20.
4. A gRNA comprising a targeting domain which binds a target domain of SEQ ID NO: 4.
5. A gRNA comprising a targeting domain which binds a target domain SEQ ID NO: 14, wherein the targeting domain does not comprise SEQ ID NO: 4.
6. A gRNA comprising a targeting domain which binds a target domain SEQ ID NO: 14, wherein the targeting domain is at least 21 nucleotides in length.
7. The gRNA of any of the preceding embodiments, wherein the targeting domain base pairs or is complementary with at least 10, 11, 12, 13, 14, 15, 16, 17, 18, 19, or 20 nucleotides of the target domain, or wherein the targeting domain comprises 0, 1, 2, or 3 mismatches with the target domain.
8. The gRNA of any of the preceding embodiments, wherein the targeting domain comprises at least 16 (e.g., 17, 18, 19, 20, 21, 22, 23, 24, 25, or 26) consecutive nucleotides of SEQ ID NO: 31.
9. The gRNA of any of the preceding embodiments, wherein the targeting domain comprises at least 16 (e.g., 17, 18, 19, 20, 21, 22, 23, 24, 25, or 26) consecutive nucleotides of SEQ ID NO: 31, and base pairs or is complementary with at least 10, 11, 12, 13, 14, 15, 16, 17, 18, 19, or 20 nucleotides of the target domain.
10. The gRNA of any of the preceding embodiments, wherein the targeting domain comprises at least 16 (e.g., 17, 18, 19, 20, 21, 22, 23, 24, 25, or 26) consecutive nucleotides of SEQ ID NO: 46.
11. The gRNA of any of the preceding embodiments, wherein the targeting domain comprises at least 16 (e.g., 17, 18, 19, 20, 21, 22, 23, 24, 25, or 26) consecutive nucleotides of SEQ ID NO: 46, and base pairs or is complementary with at least 10, 11, 12, 13, 14, 15, 16, 17, 18, 19, or 20 nucleotides of the target domain.
12. The gRNA of any of the preceding embodiments, wherein the targeting domain comprises at least 16 (e.g., 17, 18, 19, 20, 21, 22, 23, 24, 25, or 26) consecutive nucleotides of SEQ ID NO: 270.
13. The gRNA of any of the preceding embodiments, wherein the targeting domain comprises at least 16 (e.g., 17, 18, 19, 20, 21, 22, 23, 24, 25, or 26) consecutive nucleotides of SEQ ID NO: 270, and base pairs or is complementary with at least 10, 11, 12, 13, 14, 15, 16, 17, 18, 19, or 20 nucleotides of the target domain.
14. The gRNA of any of the preceding embodiments, wherein the targeting domain comprises at least 16 (e.g., 17, 18, 19, 20, 21, 22, 23, 24, 25, or 26) consecutive nucleotides of SEQ ID NO: 271.
15. The gRNA of any of the preceding embodiments, wherein the targeting domain comprises at least 16 (e.g., 17, 18, 19, 20, 21, 22, 23, 24, 25, or 26) consecutive nucleotides of SEQ ID NO: 271, and base pairs or is complementary with at least 10, 11, 12, 13, 14, 15, 16, 17, 18, 19, or 20 nucleotides of the target domain.
16. The gRNA of any of the preceding embodiments, wherein the targeting domain comprises at least 16 (e.g., 17, 18, 19, 20, 21, 22, 23, 24, 25, or 26) consecutive nucleotides of SEQ ID NO: 272.
17. The gRNA of any of the preceding embodiments, wherein the targeting domain comprises at least 16 (e.g., 17, 18, 19, 20, 21, 22, 23, 24, 25, or 26) consecutive nucleotides of SEQ ID NO: 272, and base pairs or is complementary with at least 10, 11, 12, 13, 14, 15, 16, 17, 18, 19, or 20 nucleotides of the target domain.
18. The gRNA of any of the preceding embodiments, wherein said targeting domain is configured to provide a cleavage event (e.g., a single strand break or double strand break) within the target domain, e.g., immediately after nucleotide position 10, 11, 12, 13, 14, 15, 16, 17, 18, 19, or 20 of the target domain.
19. A gRNA comprising a targeting domain, wherein the targeting domain comprises a sequence of SEQ ID NO: 21.
20. A gRNA comprising a targeting domain, wherein the targeting domain comprises a sequence of SEQ ID NO: 22.
21. A gRNA comprising a targeting domain, wherein the targeting domain comprises a sequence of SEQ ID NO: 23.
22. A gRNA comprising a targeting domain, wherein the targeting domain comprises a sequence of SEQ ID NO: 24.
23. A gRNA comprising a targeting domain, wherein the targeting domain comprises a sequence of SEQ ID NO: 25.
24. A gRNA comprising a targeting domain, wherein the targeting domain comprises a sequence of SEQ ID NO: 26.
25. A gRNA comprising a targeting domain, wherein the targeting domain comprises a sequence of SEQ ID NO: 27.
26. A gRNA comprising a targeting domain, wherein the targeting domain comprises a sequence of SEQ ID NO: 28.
27. A gRNA comprising a targeting domain, wherein the targeting domain comprises a sequence of SEQ ID NO: 29.
28. A gRNA comprising a targeting domain, wherein the targeting domain comprises a sequence of SEQ ID NO: 30.
29. A gRNA comprising a targeting domain, wherein the targeting domain comprises a sequence of SEQ ID NO: 44.
30. A gRNA comprising a targeting domain, wherein the targeting domain comprises a sequence of SEQ ID NO: 45.
31. A gRNA comprising a targeting domain, wherein the targeting domain comprises a sequence of SEQ ID NO: 119.
32. A gRNA comprising a targeting domain, wherein the targeting domain comprises a sequence of SEQ ID NO: 98.
33. A gRNA comprising a targeting domain, wherein the targeting domain comprises a sequence of Table 2 or 6.
34. A gRNA comprising a targeting domain, wherein the targeting domain comprises a sequence of Table 8 (e.g., any of SEQ ID NOS: 1-10, 40, 42, 98, or 119).
35. A gRNA comprising a targeting domain capable of directing cleavage or editing of a target domain of Table 2 (e.g., a target domain of any of SEQ ID NOS: 1-10, 40, or 42).
36. A gRNA comprising a targeting domain capable of directing cleavage or editing of a target domain of Table 6 (e.g., a target domain of any of SEQ ID NOS: 67-177).
37. A gRNA comprising a targeting domain capable of directing cleavage or editing of a target domain of Table 8 (e.g., a target domain of any of SEQ ID NOS: 1-10, 40, 42, 98, or 119).
38. The gRNA of any of the preceding embodiments, wherein the target domain is in exon 1, exon 2, exon 3, exon 4, exon 5, or exon 6 of the CLL1 sequence of SEQ ID NO: 31.
39. The gRNA of any of the preceding embodiments, wherein the target domain is in exon 1, exon2, exon 3, exon 4, exon 5, exon 6, or exon 7 of the CLL1 sequence of SEQ ID NO: 46.
40. The gRNA of any of the preceding embodiments, wherein the target domain is in exon 1, exon2, exon 3, exon 4, exon 5, exon 6, or exon 7 of the CLL1 sequence of SEQ ID NO: 270.
41. The gRNA of any of the preceding embodiments, wherein the target domain is in exon 1, exon2, exon 3, exon 4, exon 5, or exon 6 of the CLL1 sequence of SEQ ID NO: 271.
42. The gRNA of any of the preceding embodiments, wherein the target domain is in exon 1, exon2, exon 3, exon 4, or exon 5 of the CLL1 sequence of SEQ ID NO: 272.
43. The gRNA of any of the preceding embodiments, which is a single guide RNA (sgRNA).
44. The gRNA of any of the preceding embodiments, wherein the targeting domain is 16 nucleotides or more in length.
45. The gRNA of any of the preceding embodiments, wherein the targeting domain is 16, 17, 18, 19, 20, 21, 22, 23, 24, 25, or 26 nucleotides in length.
46. The gRNA of any of the preceding embodiments, wherein the targeting domain comprises a sequence of any of SEQ ID NOS: 1-10, 21-30, 40, 42, 44 or 45 or the reverse complement thereof, or a sequence having at least 90% or 95% identity to any of the foregoing, or a sequence having no more than 1, 2, or 3 mutations relative to any of the foregoing.
47. The gRNA of embodiment 46, wherein the 2 mutations are not adjacent to each other.
48. The gRNA of embodiment 46, wherein none of the 3 mutations are adjacent to each other.
49. The gRNA of any of embodiments 46-48, wherein the 1, 2, or 3 mutations are substitutions.
50. The gRNA of any of embodiments 46-48, wherein one or more of the mutations is an insertion or deletion.
51. The gRNA of any of the preceding embodiments, wherein the targeting domain comprises a sequence of any of SEQ ID NOS: 1-10, 40 or 42.
52. The gRNA of any of the preceding embodiments, wherein the targeting domain comprises a sequence of SEQ ID NO: 1.
53. The gRNA of any of the preceding embodiments, wherein the targeting domain comprises a sequence of SEQ ID NO: 2.
54. The gRNA of any of the preceding embodiments, wherein the targeting domain comprises a sequence of SEQ ID NO: 3.
55. The gRNA of any of the preceding embodiments, wherein the targeting domain comprises a sequence of SEQ ID NO: 4.
56. The gRNA of any of the preceding embodiments, wherein the targeting domain comprises a sequence of SEQ ID NO: 5.
57. The gRNA of any of the preceding embodiments, wherein the targeting domain comprises a sequence of SEQ ID NO: 6.
58. The gRNA of any of the preceding embodiments, wherein the targeting domain comprises a sequence of SEQ ID NO: 7.
59. The gRNA of any of the preceding embodiments, wherein the targeting domain comprises a sequence of SEQ ID NO: 8.
60. The gRNA of any of the preceding embodiments, wherein the targeting domain comprises a sequence of SEQ ID NO: 9.
61. The gRNA of any of the preceding embodiments, wherein the targeting domain comprises a sequence of SEQ ID NO: 10.
62. The gRNA of any of the preceding embodiments, wherein the targeting domain comprises a sequence of SEQ ID NO: 40.
63. The gRNA of any of the preceding embodiments, wherein the targeting domain comprises a sequence of SEQ ID NO: 42.
64. The gRNA of any of the preceding embodiments, wherein the targeting domain comprises a sequence of SEQ ID NO: 119.
65. The gRNA of any of the preceding embodiments, wherein the targeting domain comprises a sequence of SEQ ID NO: 98.
66. The gRNA of any of the preceding embodiments, wherein the targeting domain comprises a sequence of any of SEQ ID NOS: 1-10, 40, 42, 98, or 119.
67. The gRNA of any of the preceding embodiments, wherein the targeting domain comprises a sequence of any of SEQ ID NOS: 67-177.
68. The gRNA of any of the preceding embodiments, wherein the targeting domain comprises a sequence of any of SEQ ID NOS: 21-30 or 44-45.
69. The gRNA of any of the preceding embodiments, wherein the targeting domain comprises a sequence of SEQ ID NO: 21.
70. The gRNA of any of the preceding embodiments, wherein the targeting domain comprises a sequence of SEQ ID NO: 22.
71. The gRNA of any of the preceding embodiments, wherein the targeting domain comprises a sequence of SEQ ID NO: 23.
72. The gRNA of any of the preceding embodiments, wherein the targeting domain comprises a sequence of SEQ ID NO: 24.
73. The gRNA of any of the preceding embodiments, wherein the targeting domain comprises a sequence of SEQ ID NO: 25.
74. The gRNA of any of the preceding embodiments, wherein the targeting domain comprises a sequence of SEQ ID NO: 26.
75. The gRNA of any of the preceding embodiments, wherein the targeting domain comprises a sequence of SEQ ID NO: 27.
76. The gRNA of any of the preceding embodiments, wherein the targeting domain comprises a sequence of SEQ ID NO: 28.
77. The gRNA of any of the preceding embodiments, wherein the targeting domain comprises a sequence of SEQ ID NO: 29.
78. The gRNA of any of the preceding embodiments, wherein the targeting domain comprises a sequence of SEQ ID NO: 30.
79. The gRNA of any of the preceding embodiments, wherein the targeting domain comprises a sequence of SEQ ID NO: 44.
80. The gRNA of any of the preceding embodiments, wherein the targeting domain comprises a sequence of SEQ ID NO: 45.
81. The gRNA of any of the preceding embodiments, wherein the targeting domain comprises a sequence of SEQ ID NO: 239.
82. The gRNA of any of the preceding embodiments, wherein the targeting domain comprises a sequence of SEQ ID NO: 257.
83. The gRNA of any of the preceding embodiments, wherein the targeting domain comprises a sequence of any of SEQ ID NOs: 216-269.
84. The gRNA of any of the preceding embodiments, which comprises one or more chemical modifications (e.g., a chemical modification to a nucleobase, sugar, or backbone portion).
85. The gRNA of any of the preceding embodiments, which comprises one or more 2′O-methyl nucleotide, e.g., at a position described herein.
86. The gRNA of any of the preceding embodiments, which comprises one or more phosphorothioate or thioPACE linkage, e.g., at a position described herein.
87. The gRNA of any of the preceding embodiments, which binds a Cas9 molecule.
88. The gRNA of any one of the preceding embodiments, wherein the targeting domain is about 18-23, e.g., 20 nucleotides in length.
89. The gRNA of any of embodiments 1-88, which binds to a tracrRNA.
90. The gRNA of any of embodiments 1-88, which comprises a scaffold sequence.
91. The gRNA of any of the preceding embodiments, which comprises one or more of (e.g., all of):

a first complementarity domain;

a linking domain;

a second complementarity domain which is complementary to the first complementarity domain;

a proximal domain; and

a tail domain.

92. The gRNA of any of the preceding embodiments, which comprises a first complementarity domain.
93. The gRNA of any of the preceding embodiments, which comprises a linking domain.
94. The gRNA of embodiment 92 or 93, which comprises a second complementarity domain which is complementary to the first complementarity domain
95. The gRNA of any of the preceding embodiments, which comprises a proximal domain.
96. The gRNA of any of the preceding embodiments, which comprises a tail domain.
97. The gRNA of any of embodiments 91-96, wherein the targeting domain is heterologous to one or more of (e.g., all of):

the first complementarity domain;

the linking domain;

the second complementarity domain which is complementary to the first complementarity domain;

the proximal domain; and

the tail domain.

98. The gRNA of any of the preceding embodiments, wherein the gRNA has an editing frequency as measured by ICE of 70-100, e.g., 75-100, 80-100, 85-100, 90-100, or 95-100, or at least 70, at least 75, at least 80, at least 85, at least 90, at least 95, at least 99, or at least 100.
99. The gRNA of any of embodiments 1-97, wherein the gRNA has an editing frequency as measured by ICE of 10-90, e.g., 20-70, 25-70, 30-70, 35-70, 40-70, 45-70, 50-70, 55-70, 60-70, or 65-70, or at least 10, at least 20, at least 25, at least 30, at least 35, at least 40, at least 45, at least 50, at least 55, at least 60, at least 65, or at least 70.
100. The gRNA of any of the preceding embodiments, wherein the gRNA has an editing frequency as measured by ICE of at least 80.
101. The gRNA of any of the preceding embodiments, wherein the gRNA has an R² value of the editing frequency as measured by ICE of 0.8-1, e.g., 0.85-1, 0.9-1, 0.95-1, or at least 0.8, at least 0.85, at least 0.9, at least 0.95, at least 0.98, at least 0.99, or at least 1.
102. The gRNA of any of the preceding embodiments, wherein the gRNA has an R² value of the editing frequency as measured by ICE of at least 0.85.
103. The gRNA of any of the preceding embodiments, wherein the gRNA has an editing frequency as measured by ICE of at least 80 and an R² value of the editing frequency as measured by ICE at least 0.85.
104. The gRNA of any of the preceding embodiments, wherein the gRNA has an editing frequency, e.g., as measured by Sanger sequencing followed by ICE or TIDE analysis, of 70-100, e.g., 75-100, 80-100, 85-100, 90-100, 95-100, or at least 70, at least 75, at least 80, at least 85, at least 90, at least 95, at least 99, or at least 100.
105. The gRNA of any of the preceding embodiments, wherein the gRNA has an editing frequency, e.g., as measured by Next Generation-Targeted Amplicon Sequencing (Amplicon sequencing), of 70-100, e.g., 75-100, 80-100, 85-100, 90-100, 95-100, or at least 70, at least 75, at least 80, at least 85, at least 90, at least 95, at least 99, or at least 100.
106. A kit or composition comprising:

a) a gRNA of any of embodiments 1-105, or a nucleic acid encoding the gRNA, and

b) a second gRNA, or a nucleic acid encoding the second gRNA.

107. The kit or composition of embodiment 106, wherein the first gRNA comprises a targeting domain that comprises a sequence of GGTGGCTATTGTTTGCAGTG (SEQ ID NO: 4).
108. The kit or composition of embodiment 106 or 107, wherein the second gRNA targets a lineage-specific cell-surface antigen.
109. The kit or composition of any of embodiments 106-108 wherein the second gRNA targets a lineage-specific cell-surface antigen other than CLL1.
110. The kit or composition of any of embodiments 106-109, wherein the second gRNA targets CD33 (e.g., wherein the second gRNA comprises a targeting domain that comprises a sequence of CCCCAGGACTACTCACTCCT (SEQ ID NO: 64).
111. The kit or composition of any of embodiments 106-110, wherein the second gRNA targets CD123 (e.g., wherein the second gRNA comprises a targeting domain that comprises a sequence of TTTCTTGAGCTGCAGCTGGG (SEQ ID NO: 65) or AGTTCCCACATCCTGGTGCG (SEQ ID NO: 66)).
112. The kit or composition of any of embodiments 106-111, wherein the second gRNA comprises a targeting domain that comprises a sequence of TTTCTTGAGCTGCAGCTGGG (SEQ ID NO: 65).
113. The kit or composition of any of embodiments 106-112, wherein the second gRNA comprises a targeting domain that comprises a sequence of AGTTCCCACATCCTGGTGCG (SEQ ID NO: 66).
114. The kit or composition of any of embodiments 106-113, wherein the second gRNA comprises a targeting domain that comprises a sequence of Table A.
115. The kit or composition of any of embodiments 106-114, wherein the first gRNA comprises a targeting domain that comprises a sequence of GGTGGCTATTGTTTGCAGTG (SEQ ID NO: 4) and the second gRNA comprises a targeting domain that comprises a sequence of CCCCAGGACTACTCACTCCT (SEQ ID NO: 64).
116. The kit or composition of any of embodiments 106-115, wherein the first gRNA comprises a targeting domain that comprises a sequence of GGTGGCTATTGTTTGCAGTG (SEQ ID NO: 4) and the second gRNA comprises a targeting domain that comprises a sequence of TTTCTTGAGCTGCAGCTGGG (SEQ ID NO: 65).
117. The kit or composition of any of embodiments 106-116, wherein the first gRNA comprises a targeting domain that comprises a sequence of GGTGGCTATTGTTTGCAGTG (SEQ ID NO: 4) and the second gRNA comprises a targeting domain that comprises a sequence of AGTTCCCACATCCTGGTGCG (SEQ ID NO: 66).
118. The kit or composition of any of embodiments 106-117, which further comprises a third gRNA, or a nucleic acid encoding the third gRNA.
119. The kit or composition of embodiment 118, wherein the third gRNA targets a lineage-specific cell-surface antigen.
120. The kit or composition of embodiment 118, wherein the third gRNA targets CD33, CLL-1, or CD123.
121. The kit or composition of any of embodiments 118-120, wherein the first gRNA comprises a targeting domain that comprises a sequence of GGTGGCTATTGTTTGCAGTG (SEQ ID NO: 4), the second gRNA comprises a targeting domain that comprises a sequence of TTTCTTGAGCTGCAGCTGGG (SEQ ID NO: 65), and the third gRNA comprises a targeting domain that comprises a sequence of AGTTCCCACATCCTGGTGCG (SEQ ID NO: 66).
122. The kit or composition of any of embodiments 118-120, wherein the first gRNA comprises a targeting domain that comprises a sequence of GGTGGCTATTGTTTGCAGTG (SEQ ID NO: 4), the second gRNA comprises a targeting domain that comprises a sequence of CCCCAGGACTACTCACTCCT (SEQ ID NO: 64), and the third gRNA comprises a targeting domain that comprises a sequence of AGTTCCCACATCCTGGTGCG (SEQ ID NO: 66).
123. The kit or composition of any of embodiments 118-120, wherein the first gRNA comprises a targeting domain that comprises a sequence of GGTGGCTATTGTTTGCAGTG (SEQ ID NO: 4), the second gRNA comprises a targeting domain that comprises a sequence of TTTCTTGAGCTGCAGCTGGG (SEQ ID NO: 65), and the third gRNA comprises a targeting domain that comprises a sequence of CCCCAGGACTACTCACTCCT (SEQ ID NO: 64).
124. The kit or composition of any of embodiments 118-123, which further comprises a fourth gRNA, or a nucleic acid encoding the fourth gRNA.
125. The kit or composition of embodiment 124, wherein the fourth gRNA targets a lineage-specific cell-surface antigen.
126. The kit or composition of embodiment 124, wherein the fourth gRNA targets CD33, CLL-1, or CD123.
127. The kit or composition of any of embodiments 124-126, wherein the gRNA of (a) comprises a targeting domain that comprises a sequence of GGTGGCTATTGTTTGCAGTG (SEQ ID NO: 4), the second gRNA comprises a targeting domain that comprises a sequence of TTTCTTGAGCTGCAGCTGGG (SEQ ID NO: 65), the third gRNA comprises a targeting domain that comprises a sequence of AGTTCCCACATCCTGGTGCG (SEQ ID NO: 66), and the fourth gRNA comprises a targeting domain that comprises a sequence of CCCCAGGACTACTCACTCCT (SEQ ID NO: 64).
128. The kit or composition of any of embodiments 124-127, wherein the gRNA of (a), the second gRNA, the third gRNA, and the fourth gRNA are admixed.
129. The kit or composition of any of embodiments 124-127, wherein the gRNA of (a), the second gRNA, the third gRNA, and the fourth gRNA are in separate containers.
130. The kit or composition of any of embodiments 106-129, wherein (a) and (b) are admixed.
131. The kit or composition of any of embodiments 106-129, wherein (a) and (b) are in separate containers.
132. The kit or composition of any of embodiments 106-131, wherein the nucleic acid of (a) and the nucleic acid of (b) are part of the same nucleic acid.
133. The kit or composition of any of embodiments 106-131, wherein the nucleic acid of (a) and the nucleic acid of (b) are separate nucleic acids.
134. A genetically engineered hematopoietic cell (e.g., hematopoietic stem or progenitor cell), which comprises:

(a) a mutation at a target domain of Table 1 (e.g., a target domain of any of SEQ ID NOS: 1-20, or 40-43); and

(b) a second mutation at a gene encoding a lineage-specific cell surface antigen other than CLL1.

135. The genetically engineered hematopoietic cell of embodiment 134, wherein the mutation of (a) is at a target domain of SEQ ID NO: 1.
136. The genetically engineered hematopoietic cell of embodiment 134, wherein the mutation of (a) is at a target domain of SEQ ID NO: 2.
137. The genetically engineered hematopoietic cell of embodiment 134, wherein the mutation of (a) is at a target domain of SEQ ID NO: 3.
138. The genetically engineered hematopoietic cell of embodiment 134, wherein the mutation of (a) is at a target domain of SEQ ID NO: 4.
139. The genetically engineered hematopoietic cell of embodiment 134, wherein the mutation of (a) is at a target domain of SEQ ID NO: 5.
140. The genetically engineered hematopoietic cell of embodiment 134, wherein the mutation of (a) is at a target domain of SEQ ID NO: 6.
141. The genetically engineered hematopoietic cell of embodiment 134, wherein the mutation of (a) is at a target domain of SEQ ID NO: 7.
142. The genetically engineered hematopoietic cell of embodiment 134, wherein the mutation of (a) is at a target domain of SEQ ID NO: 8.
143. The genetically engineered hematopoietic cell of embodiment 134, wherein the mutation of (a) is at a target domain of SEQ ID NO: 9.
144. The genetically engineered hematopoietic cell of embodiment 134, wherein the mutation of (a) is at a target domain of SEQ ID NO: 10.
145. The genetically engineered hematopoietic cell of embodiment 134, wherein the mutation of (a) is at a target domain of SEQ ID NO: 40.
146. The genetically engineered hematopoietic cell of embodiment 134, wherein the mutation of (a) is at a target domain of SEQ ID NO: 42.
147. The genetically engineered hematopoietic cell of embodiment 134, wherein the mutation of (a) is at a target domain of SEQ ID NO: 98.
148. The genetically engineered hematopoietic cell of embodiment 134, wherein the mutation of (a) is at a target domain of SEQ ID NO: 119.
149. The genetically engineered hematopoietic cell of embodiment 134, wherein the mutation of (a) is at a target domain of any of Tables 2 or 6.
150. The genetically engineered hematopoietic cell of any of embodiments 134-149, wherein the mutation of (a) comprises an insertion, a deletion, or a substitution (e.g., a single nucleotide variant).
151. The genetically engineered hematopoietic cell of any of embodiments 134-149, which comprises an insertion of 1 nt, or a deletion of 1 nt, 2 nt, or 3 nt, or 4 nt in CLL1.
152. The genetically engineered hematopoietic cell of any of embodiments 134-151, which comprises an indel as described herein, e.g., an indel produced by or producible by a gRNA described herein (e.g., any of gRNA D, gRNA F, gRNA G, gRNA 02, or gRNA P2).
153. The genetically engineered hematopoietic cell of any of embodiments 134-152, which comprises an indel produced by or producible by a CRISPR system described herein, e.g., a method of Example 1, 2, 3, 4, or 5.
154. The genetically engineered cell of any of embodiments 150-153, wherein the deletion is fully within the target domain of any of SEQ ID NOS: 1-10.
155. The genetically engineered cell of embodiment 154, wherein the deletion is 1, 2, 4, 5, 7, 8, 10, 11, 13, 14, 16, or 17 nucleotides in length.
156. The genetically engineered cell of any of embodiments 150-153, wherein the deletion has one or both endpoints outside of the target domain of any of SEQ ID NOS: 1-10.
157. The genetically engineered cell of any of embodiments 134-156, wherein the mutation results in a frameshift.
158. The genetically engineered hematopoietic cell of any of embodiments 134-157, wherein the second mutation comprises an insertion, a deletion, or a substitution (e.g., a single nucleotide variant).
159. Use of a gRNA of any of embodiments 1-105, or gRNAs of a composition or kit of any of embodiments 106-133, for reducing expression of CLL1 in a sample of hematopoietic cells stem or progenitor cells using a CRISPR/Cas9 system.
160. Use of a CRISPR/Cas9 system for reducing expression of CLL1 in a sample of hematopoietic cells stem or progenitor cells, wherein the gRNA of the CRISPR/Cas9 system is a gRNA of any of embodiments 1-105 or gRNAs of a composition or kit of any of embodiments 106-133.
161. A method of producing a genetically engineered cell, comprising:

(i) providing a cell (e.g., a hematopoietic stem or progenitor cell, e.g., a wild-type hematopoietic stem or progenitor cell), and

(ii) introducing into the cell (a) a guide RNA (gRNA) of any of embodiments 1-105 or gRNAs of a composition or kit of any of embodiments 106-133; and (b) an endonuclease that binds the gRNA (e.g., a Cas9 molecule), thereby producing the genetically engineered cell.

162. A method of producing a genetically engineered cell, comprising:

(i) providing a cell (e.g., a hematopoietic stem or progenitor cell, e.g., a wild-type hematopoietic stem or progenitor cell), and

(ii) introducing into the cell (a) a gRNA of any of embodiments 1-105 or gRNAs of a composition or kit of any of embodiments 106-133; and (b) a Cas9 molecule that binds the gRNA, thereby producing the genetically engineered cell.

163. The method or use of any of embodiments 159-162, which results in the genetically engineered hematopoietic stem or progenitor cell having a reduced expression level of CLL1 as compared with a wild-type counterpart cell.
164. The method or use of any of embodiments 159-163, which results in the genetically engineered hematopoietic stem or progenitor cell having a reduced expression level of CLL1 that is less than 20% of the level of CLL1 in a wild-type counterpart cell.
165. The method or use of any of embodiments 159-164, which is performed on a plurality of hematopoietic stem or progenitor cells.
166. The method or use of any of embodiments 159-165, which is performed on a cell population comprising a plurality of hematopoietic stem cells and a plurality of hematopoietic progenitor cells.
167. The method or use of any of embodiments 159-166, which produces a cell population according to any of embodiments 285-383.
168. The method of any of embodiments 161-167, wherein the nucleic acids of (a) and (b) are encoded on one vector, which is introduced into the cell.
169. The method of embodiment 168, wherein the vector is a viral vector.
170. The method of embodiment 169, wherein (a) and (b) are introduced into the cell as a pre-formed ribonucleoprotein complex.
171. The method of embodiment 170, wherein the ribonucleoprotein complex is introduced into the cell via electroporation.
172. The method of any of any of embodiments 161-168, wherein the endonuclease (e.g., a Cas9 molecule) is introduced into the cell by delivering into the cell a nucleic acid molecule (e.g., an mRNA molecule or a viral vector, e.g., AAV) encoding the endonuclease.
173. The method of any of embodiments 161-172, wherein the cell (e.g., the hematopoietic stem or progenitor cell) is CD34+.
174. The method of any of embodiments 161-173, wherein cell viability of a population of the cells is at least 80%, 90%, 95%, or 98% of the cell viability of control cells (e.g., mock electroporated cells) with 48 hours after introduction of the gRNA into the cells.
175. The method of any of embodiments 161-174, wherein at least 80%, 85%, 90%, 95%, or 98% of cells in the population are viable 48 hours after introduction of the gRNA into the cells.
176. The method of any of embodiments 161-175, wherein the hematopoietic stem or progenitor cell is from bone marrow cells or peripheral blood mononuclear cells (PBMCs) of a subject.
177. The method of any of embodiments 161-176, wherein the subject has a hematopoietic disorder, e.g., a hematopoietic malignancy, e.g., a leukemia, e.g., AML.
178. The method of any of embodiments 161-176, wherein the subject has a hematological disorder, e.g., a precancerous condition, e.g., a myelodysplasia, a myelodysplastic syndrome (MDS), or a preleukemia.
179. The method of any of embodiments 161-177, wherein the subject has a cancer, wherein cells of the cancer express CLL1 (e.g., wherein at least a plurality of the cancer cells express CLL1).
180. The method or use of any of embodiments 159-179, which results in a mutation that causes a reduced expression level of CLL1 as compared with a wild-type counterpart cell.
181. The method or use of any of embodiments 159-180, which results in a mutation that causes a reduced expression level of wild-type CLL1 as compared with a wild-type counterpart cell.
182. The method or use of any of embodiments 159-181, which produces a genetically engineered hematopoietic stem or progenitor cell having a reduced expression level of CLL1 as compared with a wild-type counterpart cell.
183. The method or use of any of embodiments 159-182, which produces a genetically engineered hematopoietic stem or progenitor cell having a reduced expression level of wild-type CLL1 as compared with a wild-type counterpart cell.
184. A genetically engineered hematopoietic stem or progenitor cell, which is produced by a method of any of embodiments 159-183.
185. A nucleic acid (e.g., DNA) encoding the gRNA of any of embodiments 1-105.
186. A genetically engineered cell (e.g., a hematopoietic stem or progenitor cell), which comprises a mutation at a target domain of Table 1 (e.g., a target domain of any of SEQ ID NOS: 1-20), e.g., wherein the mutation is a result of the genetic engineering.
187. A genetically engineered cell (e.g., a hematopoietic stem or progenitor cell), which comprises a mutation at a target domain of Table 8 (e.g., a target domain of any of SEQ ID NOS: 1-10, 40, 42, 98, or 119), e.g., wherein the mutation is a result of the genetic engineering.
188. A genetically engineered cell (e.g., a hematopoietic stem or progenitor cell), which comprises a mutation at a target domain of Table 6 (e.g., a target domain of any of SEQ ID NOS: 67-177), e.g., wherein the mutation is a result of the genetic engineering.
189. A genetically engineered cell (e.g., a hematopoietic stem or progenitor cell), which comprises a mutation within 100, 90, 80, 70, 60, 50, 40, 30, 20, or 10 nucleotides (upstream or downstream) of a target domain of Table 1 (e.g., a target domain of any of SEQ ID NOS: 1-20 or 40-43).
190. A genetically engineered cell (e.g., a hematopoietic stem or progenitor cell), which comprises a mutation within 100, 90, 80, 70, 60, 50, 40, 30, 20, or 10 nucleotides (upstream or downstream) of a target domain of Table 6 (e.g., a target domain of any of SEQ ID NOS: 67-177).
191. A genetically engineered cell (e.g., a hematopoietic stem or progenitor cell), which comprises a mutation within 100, 90, 80, 70, 60, 50, 40, 30, 20, or 10 nucleotides (upstream or downstream) of a target domain of Table 8 (e.g., a target domain of any of SEQ ID NOS: 1-10, 40, 42, 98, or 119).
192. The genetically engineered cell of embodiment 198, wherein the mutation is within 100, 90, 80, 70, 60, 50, 40, 30, 20, or 10 nucleotides (upstream or downstream) of any of SEQ ID NOS: 4, 6, or 7.
193. The genetically engineered cell of embodiment 198, wherein the mutation is within 100, 90, 80, 70, 60, 50, 40, 30, 20, or 10 nucleotides downstream of SEQ ID NO: 4.
194. The genetically engineered cell of embodiment 198, wherein the mutation is within 100, 90, 80, 70, 60, 50, 40, 30, 20, or 10 nucleotides upstream of SEQ ID NO: 4.
195. A genetically engineered hematopoietic stem or progenitor cell, which comprises a mutation at a target domain of SEQ ID NO: 1.
196. A genetically engineered hematopoietic stem or progenitor cell, which comprises a mutation at a target domain of SEQ ID NO: 1, wherein the mutation results in a reduced expression level of CLL1 as compared with a wild-type counterpart cell.
197. A genetically engineered hematopoietic stem or progenitor cell, which comprises a mutation at a target domain of SEQ ID NO: 1, wherein the mutation results in a reduced expression level of CLL1 that is less than 20% of the level of CLL1 in a wild-type counterpart cell.
198. A genetically engineered hematopoietic stem or progenitor cell, which comprises a mutation at a target domain of SEQ ID NO: 2.
199. A genetically engineered hematopoietic stem or progenitor cell, which comprises a mutation at a target domain of SEQ ID NO: 2, wherein the mutation results in a reduced expression level of CLL1 as compared with a wild-type counterpart cell.
200. A genetically engineered hematopoietic stem or progenitor cell, which comprises a mutation at a target domain of SEQ ID NO: 2, wherein the mutation results in a reduced expression level of CLL1 that is less than 20% of the level of CLL1 in a wild-type counterpart cell.
201. A genetically engineered hematopoietic stem or progenitor cell, which comprises a mutation at a target domain of SEQ ID NO: 3.
202. A genetically engineered hematopoietic stem or progenitor cell, which comprises a mutation at a target domain of SEQ ID NO: 3, wherein the mutation results in a reduced expression level of CLL1 as compared with a wild-type counterpart cell.
203. A genetically engineered hematopoietic stem or progenitor cell, which comprises a mutation at a target domain of SEQ ID NO: 3, wherein the mutation results in a reduced expression level of CLL1 that is less than 20% of the level of CLL1 in a wild-type counterpart cell.
204. A genetically engineered hematopoietic stem or progenitor cell, which comprises a mutation at a target domain of SEQ ID NO: 4.
205. A genetically engineered hematopoietic stem or progenitor cell, which comprises a mutation at a target domain of SEQ ID NO: 4, wherein the mutation results in a reduced expression level of CLL1 as compared with a wild-type counterpart cell.
206. A genetically engineered hematopoietic stem or progenitor cell, which comprises a mutation at a target domain of SEQ ID NO: 4, wherein the mutation results in a reduced expression level of CLL1 that is less than 20% of the level of CLL1 in a wild-type counterpart cell.
207. A genetically engineered hematopoietic stem or progenitor cell, which comprises a mutation at a target domain of SEQ ID NO: 5.
208. A genetically engineered hematopoietic stem or progenitor cell, which comprises a mutation at a target domain of SEQ ID NO: 5, wherein the mutation results in a reduced expression level of CLL1 as compared with a wild-type counterpart cell.
209. A genetically engineered hematopoietic stem or progenitor cell, which comprises a mutation at a target domain of SEQ ID NO: 5, wherein the mutation results in a reduced expression level of CLL1 that is less than 20% of the level of CLL1 in a wild-type counterpart cell.
210. A genetically engineered hematopoietic stem or progenitor cell, which comprises a mutation at a target domain of SEQ ID NO: 6.
211. A genetically engineered hematopoietic stem or progenitor cell, which comprises a mutation at a target domain of SEQ ID NO: 6, wherein the mutation results in a reduced expression level of CLL1 as compared with a wild-type counterpart cell.
212. A genetically engineered hematopoietic stem or progenitor cell, which comprises a mutation at a target domain of SEQ ID NO: 6, wherein the mutation results in a reduced expression level of CLL1 that is less than 20% of the level of CLL1 in a wild-type counterpart cell.
213. A genetically engineered hematopoietic stem or progenitor cell, which comprises a mutation at a target domain of SEQ ID NO: 7.
214. A genetically engineered hematopoietic stem or progenitor cell, which comprises a mutation at a target domain of SEQ ID NO: 7, wherein the mutation results in a reduced expression level of CLL1 as compared with a wild-type counterpart cell.
215. A genetically engineered hematopoietic stem or progenitor cell, which comprises a mutation at a target domain of SEQ ID NO: 7, wherein the mutation results in a reduced expression level of CLL1 that is less than 20% of the level of CLL1 in a wild-type counterpart cell.
216. A genetically engineered hematopoietic stem or progenitor cell, which comprises a mutation at a target domain of SEQ ID NO: 8.
217. A genetically engineered hematopoietic stem or progenitor cell, which comprises a mutation at a target domain of SEQ ID NO: 8, wherein the mutation results in a reduced expression level of CLL1 as compared with a wild-type counterpart cell.
218. A genetically engineered hematopoietic stem or progenitor cell, which comprises a mutation at a target domain of SEQ ID NO: 8, wherein the mutation results in a reduced expression level of CLL1 that is less than 20% of the level of CLL1 in a wild-type counterpart cell.
219. A genetically engineered hematopoietic stem or progenitor cell, which comprises a mutation at a target domain of SEQ ID NO: 9.
220. A genetically engineered hematopoietic stem or progenitor cell, which comprises a mutation at a target domain of SEQ ID NO: 9, wherein the mutation results in a reduced expression level of CLL1 as compared with a wild-type counterpart cell.
221. A genetically engineered hematopoietic stem or progenitor cell, which comprises a mutation at a target domain of SEQ ID NO: 9, wherein the mutation results in a reduced expression level of CLL1 that is less than 20% of the level of CLL1 in a wild-type counterpart cell.
222. A genetically engineered hematopoietic stem or progenitor cell, which comprises a mutation at a target domain of SEQ ID NO: 10.
223. A genetically engineered hematopoietic stem or progenitor cell, which comprises a mutation at a target domain of SEQ ID NO: 10, wherein the mutation results in a reduced expression level of CLL1 as compared with a wild-type counterpart cell.
224. A genetically engineered hematopoietic stem or progenitor cell, which comprises a mutation at a target domain of SEQ ID NO: 10, wherein the mutation results in a reduced expression level of CLL1 that is less than 20% of the level of CLL1 in a wild-type counterpart cell.
225. A genetically engineered hematopoietic stem or progenitor cell, which comprises a mutation at a target domain of SEQ ID NO: 40.
226. A genetically engineered hematopoietic stem or progenitor cell, which comprises a mutation at a target domain of SEQ ID NO: 40, wherein the mutation results in a reduced expression level of CLL1 as compared with a wild-type counterpart cell.
227. A genetically engineered hematopoietic stem or progenitor cell, which comprises a mutation at a target domain of SEQ ID NO: 40, wherein the mutation results in a reduced expression level of CLL1 that is less than 20% of the level of CLL1 in a wild-type counterpart cell.
228. A genetically engineered hematopoietic stem or progenitor cell, which comprises a mutation at a target domain of SEQ ID NO: 42.
229. A genetically engineered hematopoietic stem or progenitor cell, which comprises a mutation at a target domain of SEQ ID NO: 42, wherein the mutation results in a reduced expression level of CLL1 as compared with a wild-type counterpart cell.
230. A genetically engineered hematopoietic stem or progenitor cell, which comprises a mutation at a target domain of SEQ ID NO: 42, wherein the mutation results in a reduced expression level of CLL1 that is less than 20% of the level of CLL1 in a wild-type counterpart cell.
231. A genetically engineered hematopoietic stem or progenitor cell, which comprises a mutation at a target domain of SEQ ID NO: 119.
232. A genetically engineered hematopoietic stem or progenitor cell, which comprises a mutation at a target domain of SEQ ID NO: 119, wherein the mutation results in a reduced expression level of CLL1 as compared with a wild-type counterpart cell.
233. A genetically engineered hematopoietic stem or progenitor cell, which comprises a mutation at a target domain of SEQ ID NO: 119, wherein the mutation results in a reduced expression level of CLL1 that is less than 20% of the level of CLL1 in a wild-type counterpart cell.
234. A genetically engineered hematopoietic stem or progenitor cell, which comprises a mutation at a target domain of SEQ ID NO: 98.
235. A genetically engineered hematopoietic stem or progenitor cell, which comprises a mutation at a target domain of SEQ ID NO: 98, wherein the mutation results in a reduced expression level of CLL1 as compared with a wild-type counterpart cell.
236. A genetically engineered hematopoietic stem or progenitor cell, which comprises a mutation at a target domain of SEQ ID NO: 98, wherein the mutation results in a reduced expression level of CLL1 that is less than 20% of the level of CLL1 in a wild-type counterpart cell.
237. The genetically engineered cell of any of embodiments 186-236, comprising a predicted off target site which does not comprise a mutation or sequence change relative to the sequence of the site prior to gene editing of CLL1.
238. The genetically engineered cell of any of embodiments 186-237, comprising two predicted off target sites which do not comprise a mutation or sequence change relative to the sequence of the site prior to gene editing of CLL1.
239. The genetically engineered cell of any of embodiments 186-238, comprising at least 3, 4, 5, 6, 7, 8, 9, 10, 11, 12, 13, 14, 15, 16, 17, 18, 19, or 20 predicted off target sites which do not comprise a mutation or sequence change relative to the sequence of the site prior to gene editing of CLL1.
240. The genetically engineered cell of any of the preceding embodiments, which does not comprise a mutation in any predicted off-target site, e.g., in any site in the human genome having 1, 2, 3, or 4 mismatches relative to the target domain.
241. The genetically engineered cell of any of the preceding embodiments, which does not comprise a mutation in any site in the human genome having 1 mismatch relative to the target domain.
242. The genetically engineered cell of any of the preceding embodiments, which does not comprise a mutation in any site in the human genome having 1 or 2 mismatches relative to the target domain.
243. The genetically engineered cell of any of the preceding embodiments, which does not comprise a mutation in any site in the human genome having 1, 2, or 3 mismatches relative to the target domain.
244. The genetically engineered cell of any of the preceding embodiments, which does not comprise a mutation in any site in the human genome having 1, 2, 3, or 4 mismatches relative to the target domain.
245. The genetically engineered cell of any of embodiments 184 or 186-244, wherein the mutation comprises an insertion, a deletion, or a substitution (e.g., a single nucleotide variant).
246. The genetically engineered cell of embodiment 245, wherein the deletion is fully within the target domain of any of SEQ ID NOS: 1-20, 40-43, 98, or 119.
247. The genetically engineered cell of embodiment 246, wherein the deletion is 1, 2, 4, 5, 7, 8, 10, 11, 13, 14, 16, or 17 nucleotides in length.
248. The genetically engineered cell of embodiment 167, wherein the deletion has one or both endpoints outside of the target domain of any of SEQ ID NOS: 1-20, 40-43, 98, or 119.
249. The genetically engineered cell of any of embodiments 184 or 186-248, wherein the mutation results in a frameshift.
250. The genetically engineered cell (e.g., hematopoietic stem or progenitor cell) of any of embodiments 184 or 186-249, wherein the mutation results in a reduced expression level of wild-type CLL1 as compared with a wild-type counterpart cell (e.g., less than 50%, 40%, 30%, 20%, 15%, 10%, or 5% of the level in the wild-type counterpart cell).
251. The genetically engineered cell of any of embodiments 184 or 186-250, wherein the cell has a reduced level of wild-type CLL1 protein as compared with a wild-type counterpart cell (e.g., less than 50%, 40%, 30%, 20%, 15%, 10%, or 5% of the level in the wild-type counterpart cell).
252. The genetically engineered cell of any of embodiments 184 or 186-251, which does not express CLL1.
253. The genetically engineered cell (e.g., hematopoietic stem or progenitor cell) of any of the preceding embodiments, wherein the mutation results in a lack of expression of CLL1.
254. The genetically engineered cell (e.g., hematopoietic stem or progenitor cell) of any of the preceding embodiments, which expresses less than 20% of the CLL1 expressed by a wild-type counterpart cell.
255. The genetically engineered cell (e.g., hematopoietic stem or progenitor cell) of any of the preceding embodiments, wherein the reduced expression level of CLL1 is in a cell differentiated from (e.g., terminally differentiated from) the hematopoietic stem or progenitor cell, and the wild-type counterpart cell is a cell differentiated from (e.g., terminally differentiated from) a wild-type hematopoietic stem or progenitor cell.
256. The genetically engineered cell (e.g., hematopoietic stem or progenitor cell) of embodiment 255, wherein the cell differentiated from the hematopoietic stem or progenitor cell is a myeloblast, monocyte, or myeloid dendritic cell.
257. The genetically engineered cell of any of embodiments 184 or 186-256, which is CD34+.
258. The genetically engineered cell of any of embodiments 184 or 186-257, which is from bone marrow cells or peripheral blood mononuclear cells of a subject.
259. The genetically engineered cell of embodiment 258, wherein the subject is a human patient having a hematopoietic malignancy, e.g., AML.
260. The genetically engineered cell of embodiment 258, wherein the subject is a human patient having a hematological disorder, e.g., a precancerous condition, e.g., a myelodysplasia, a myelodysplastic syndrome (MDS), or a preleukemia.
261. The genetically engineered cell of any one of embodiments 258-260, wherein the subject has a cancer, wherein cells of the cancer express CLL1 (e.g., wherein at least a plurality of the cancer cells express CLL1).
262. The genetically engineered cell of embodiment 258, wherein the subject is a healthy human donor (e.g., an HLA-matched donor).
263. The genetically engineered cell of any of embodiments 184 or 186-262, which further comprises a nuclease chosen from a CRISPR endonuclease, a zinc finger nuclease (ZFN), a transcription activator-like effector-based nuclease (TALEN), or a meganuclease, or a nucleic acid (e.g., DNA or RNA) encoding the nuclease, wherein optionally the nuclease is specific for CLL1.
264. The genetically engineered cell of any of embodiments 184 or 186-263, which further comprises a gRNA (e.g., a single guide RNA) specific for CLL1, or a nucleic acid encoding the gRNA.
265. The genetically engineered cell of embodiment 264, wherein the gRNA is a gRNA described herein, e.g., a gRNA of any of embodiments 1-105.
266. The genetically engineered cell of any of embodiments 184 or 186-265, which was made by a process comprising contacting the cell with a nuclease chosen from a CRISPR endonuclease, a zinc finger nuclease (ZFN), a transcription activator-like effector-based nuclease (TALEN), or a meganuclease (e.g., by contacting the cell with the nuclease or a nucleic acid encoding the nuclease).
267. The genetically engineered cell of any of embodiments 184 or 186-266, which was made by a process comprising contacting the cell with a nickase or a catalytically inactive Cas9 molecule (dCas9), e.g., fused to a function domain (e.g., by contacting the cell with the nuclease or a nucleic acid encoding the nuclease).
268. The genetically engineered cell of any of embodiments 184 or 186-267, in which both copies of CLL1 are mutant.
269. The genetically engineered cell of embodiment 286, wherein both copies of CLL1 have the same mutation.
270. The genetically engineered cell of embodiment 286, wherein the copies of CLL1 have different mutations.
271. The genetically engineered cell of any of embodiments 184 or 186-186, comprising a first copy of CLL1 having a first mutation and a second copy of CLL1 having a second mutation, wherein the first and second mutations are different.
272. The genetically engineered cell of embodiment 271, wherein the first copy of CLL1 comprises a first deletion.
273. The genetically engineered cell of embodiment 271 or 272, wherein the second copy of CLL1 comprises a second deletion.
274. The genetically engineered cell of any of embodiments 271-273, wherein the first and second deletions overlap.
275. The genetically engineered cell of any of embodiments 271-274, wherein an endpoint of the first deletion is within the second deletion.
276. The genetically engineered cell of any of embodiments 271-275, wherein both endpoints of the first deletion are within the second deletion.
277. The genetically engineered cell of any of embodiments 271-273, wherein the first and second deletion share an endpoint.
278. The genetically engineered cell of any of embodiments 184 or 186-277, wherein the first and second mutations are each independently selected from: an insertion of 1 nt, or a deletion of 1 nt, 2 nt, 3 nt, or 4 nt.
279. The genetically engineered cell of any of embodiments 184 or 186-278, which is capable of forming a BFU-E colony, a CFU-G colony, a CFU-M colony, a CFU-GM colony, or a CFU-GEMM colony.
280. The genetically engineered cell of any of embodiments 184 or 186-279, which is capable of producing a cytokine, e.g., an inflammatory cytokine, e.g., IL-6, TNF-α, IL-1β, or MIP-1α.
281. The genetically engineered cell of any of embodiments 184 or 186-280, which is capable of producing a cytokine, e.g., an inflammatory cytokine, e.g., IL-6, TNF-α, IL-1β, or MIP-1α, at a level comparable to an otherwise similar cell that is CLL1 wildtype.
282. The genetically engineered cell of any of embodiments 184 or 186-281, which is capable of producing a cytokine, e.g., an inflammatory cytokine, e.g., IL-6, TNF-α, IL-1β, or MIP-1α, at a level that is at least 70%, 80%, 85%, 90%, or 95% of the levels produced by an otherwise similar cell that is CLL1 wildtype.
283. The genetically engineered cell of any of embodiments 280-282, which is capable of producing the cytokine when simulated with a TLR agonist, e.g., LPS or R848, e.g., as described in Example 5.
284. The genetically engineered cell of any of embodiments 280-283, which is capable of phagocytosis.
285. A cell population, comprising a plurality of the genetically engineered hematopoietic stem or progenitor cells of any embodiments 184 or 186-284 (e.g., comprising hematopoietic stem cells, hematopoietic progenitor cells, or a combination thereof).
286. A cell population, comprising a plurality of genetically engineered hematopoietic stem or progenitor cells which comprise a mutation at a target domain of SEQ ID NO: 1.
287. A cell population, comprising a plurality of genetically engineered hematopoietic stem or progenitor cells which comprise a mutation at a target domain of SEQ ID NO: 1, wherein the mutation results in a reduced expression level of CLL1 as compared with a wild-type counterpart cell population.
288. A cell population, comprising a plurality of genetically engineered hematopoietic stem or progenitor cells which comprise a mutation at a target domain of SEQ ID NO: 1, wherein the mutation results in a reduced expression level of CLL1 that is less than 20% of the level of CLL1 in a wild-type counterpart cell population.
289. A cell population, comprising a plurality of genetically engineered hematopoietic stem or progenitor cells which comprise a mutation at a target domain of SEQ ID NO: 2.
290. A cell population, comprising a plurality of genetically engineered hematopoietic stem or progenitor cells which comprise a mutation at a target domain of SEQ ID NO: 2, wherein the mutation results in a reduced expression level of CLL1 as compared with a wild-type counterpart cell population.
291. A cell population, comprising a plurality of genetically engineered hematopoietic stem or progenitor cells which comprise a mutation at a target domain of SEQ ID NO: 2, wherein the mutation results in a reduced expression level of CLL1 that is less than 20% of the level of CLL1 in a wild-type counterpart cell population.
292. A cell population, comprising a plurality of genetically engineered hematopoietic stem or progenitor cells which comprise a mutation at a target domain of SEQ ID NO: 3.
293. A cell population, comprising a plurality of genetically engineered hematopoietic stem or progenitor cells which comprise a mutation at a target domain of SEQ ID NO: 3, wherein the mutation results in a reduced expression level of CLL1 as compared with a wild-type counterpart cell population.
294. A cell population, comprising a plurality of genetically engineered hematopoietic stem or progenitor cells which comprise a mutation at a target domain of SEQ ID NO: 3, wherein the mutation results in a reduced expression level of CLL1 that is less than 20% of the level of CLL1 in a wild-type counterpart cell population.
295. A cell population, comprising a plurality of genetically engineered hematopoietic stem or progenitor cells which comprise a mutation at a target domain of SEQ ID NO: 4.
296. A cell population, comprising a plurality of genetically engineered hematopoietic stem or progenitor cells which comprise a mutation at a target domain of SEQ ID NO: 4, wherein the mutation results in a reduced expression level of CLL1 as compared with a wild-type counterpart cell population.
297. A cell population, comprising a plurality of genetically engineered hematopoietic stem or progenitor cells which comprise a mutation at a target domain of SEQ ID NO: 4, wherein the mutation results in a reduced expression level of CLL1 that is less than 20% of the level of CLL1 in a wild-type counterpart cell population.
298. A cell population, comprising a plurality of genetically engineered hematopoietic stem or progenitor cells which comprise a mutation at a target domain of SEQ ID NO: 5.
299. A cell population, comprising a plurality of genetically engineered hematopoietic stem or progenitor cells which comprise a mutation at a target domain of SEQ ID NO: 5, wherein the mutation results in a reduced expression level of CLL1 as compared with a wild-type counterpart cell population.
300. A cell population, comprising a plurality of genetically engineered hematopoietic stem or progenitor cells which comprise a mutation at a target domain of SEQ ID NO: 5, wherein the mutation results in a reduced expression level of CLL1 that is less than 20% of the level of CLL1 in a wild-type counterpart cell population.
301. A cell population, comprising a plurality of genetically engineered hematopoietic stem or progenitor cells which comprise a mutation at a target domain of SEQ ID NO: 6.
302. A cell population, comprising a plurality of genetically engineered hematopoietic stem or progenitor cells which comprise a mutation at a target domain of SEQ ID NO: 6, wherein the mutation results in a reduced expression level of CLL1 as compared with a wild-type counterpart cell population.
303. A cell population, comprising a plurality of genetically engineered hematopoietic stem or progenitor cells which comprise a mutation at a target domain of SEQ ID NO: 6, wherein the mutation results in a reduced expression level of CLL1 that is less than 20% of the level of CLL1 in a wild-type counterpart cell population.
304. A cell population, comprising a plurality of genetically engineered hematopoietic stem or progenitor cells which comprise a mutation at a target domain of SEQ ID NO: 7.
305. A cell population, comprising a plurality of genetically engineered hematopoietic stem or progenitor cells which comprise a mutation at a target domain of SEQ ID NO: 7, wherein the mutation results in a reduced expression level of CLL1 as compared with a wild-type counterpart cell population.
306. A cell population, comprising a plurality of genetically engineered hematopoietic stem or progenitor cells which comprise a mutation at a target domain of SEQ ID NO: 7, wherein the mutation results in a reduced expression level of CLL1 that is less than 20% of the level of CLL1 in a wild-type counterpart cell population.
307. A cell population, comprising a plurality of genetically engineered hematopoietic stem or progenitor cells which comprise a mutation at a target domain of SEQ ID NO: 8.
308. A cell population, comprising a plurality of genetically engineered hematopoietic stem or progenitor cells which comprise a mutation at a target domain of SEQ ID NO: 8, wherein the mutation results in a reduced expression level of CLL1 as compared with a wild-type counterpart cell population.
309. A cell population, comprising a plurality of genetically engineered hematopoietic stem or progenitor cells which comprise a mutation at a target domain of SEQ ID NO: 8, wherein the mutation results in a reduced expression level of CLL1 that is less than 20% of the level of CLL1 in a wild-type counterpart cell population.
310. A cell population, comprising a plurality of genetically engineered hematopoietic stem or progenitor cells which comprise a mutation at a target domain of SEQ ID NO: 9.
311. A cell population, comprising a plurality of genetically engineered hematopoietic stem or progenitor cells which comprise a mutation at a target domain of SEQ ID NO: 9, wherein the mutation results in a reduced expression level of CLL1 as compared with a wild-type counterpart cell population.
312. A cell population, comprising a plurality of genetically engineered hematopoietic stem or progenitor cells which comprise a mutation at a target domain of SEQ ID NO: 9, wherein the mutation results in a reduced expression level of CLL1 that is less than 20% of the level of CLL1 in a wild-type counterpart cell population.
313. A cell population, comprising a plurality of genetically engineered hematopoietic stem or progenitor cells which comprise a mutation at a target domain of SEQ ID NO: 10.
314. A cell population, comprising a plurality of genetically engineered hematopoietic stem or progenitor cells which comprise a mutation at a target domain of SEQ ID NO: 10, wherein the mutation results in a reduced expression level of CLL1 as compared with a wild-type counterpart cell population.
315. A cell population, comprising a plurality of genetically engineered hematopoietic stem or progenitor cells which comprise a mutation at a target domain of SEQ ID NO: 10, wherein the mutation results in a reduced expression level of CLL1 that is less than 20% of the level of CLL1 in a wild-type counterpart cell population.
316. A cell population, comprising a plurality of genetically engineered hematopoietic stem or progenitor cells which comprise a mutation at a target domain of SEQ ID NO: 40.
317. A cell population, comprising a plurality of genetically engineered hematopoietic stem or progenitor cells which comprise a mutation at a target domain of SEQ ID NO: 40, wherein the mutation results in a reduced expression level of CLL1 as compared with a wild-type counterpart cell population.
318. A cell population, comprising a plurality of genetically engineered hematopoietic stem or progenitor cells which comprise a mutation at a target domain of SEQ ID NO: 40, wherein the mutation results in a reduced expression level of CLL1 that is less than 20% of the level of CLL1 in a wild-type counterpart cell population.
319. A cell population, comprising a plurality of genetically engineered hematopoietic stem or progenitor cells which comprise a mutation at a target domain of SEQ ID NO: 42.
320. A cell population, comprising a plurality of genetically engineered hematopoietic stem or progenitor cells which comprise a mutation at a target domain of SEQ ID NO: 42, wherein the mutation results in a reduced expression level of CLL1 as compared with a wild-type counterpart cell population.
321. A cell population, comprising a plurality of genetically engineered hematopoietic stem or progenitor cells which comprise a mutation at a target domain of SEQ ID NO: 42, wherein the mutation results in a reduced expression level of CLL1 that is less than 20% of the level of CLL1 in a wild-type counterpart cell population.
322. A cell population, comprising a plurality of genetically engineered hematopoietic stem or progenitor cells which comprise a mutation at a target domain of SEQ ID NO: 98.
323. A cell population, comprising a plurality of genetically engineered hematopoietic stem or progenitor cells which comprise a mutation at a target domain of SEQ ID NO: 98, wherein the mutation results in a reduced expression level of CLL1 as compared with a wild-type counterpart cell population.
324. A cell population, comprising a plurality of genetically engineered hematopoietic stem or progenitor cells which comprise a mutation at a target domain of SEQ ID NO: 98, wherein the mutation results in a reduced expression level of CLL1 that is less than 20% of the level of CLL1 in a wild-type counterpart cell population.
325. A cell population, comprising a plurality of genetically engineered hematopoietic stem or progenitor cells which comprise a mutation at a target domain of SEQ ID NO: 119.
326. A cell population, comprising a plurality of genetically engineered hematopoietic stem or progenitor cells which comprise a mutation at a target domain of SEQ ID NO: 119, wherein the mutation results in a reduced expression level of CLL1 as compared with a wild-type counterpart cell population.
327. A cell population, comprising a plurality of genetically engineered hematopoietic stem or progenitor cells which comprise a mutation at a target domain of SEQ ID NO: 119, wherein the mutation results in a reduced expression level of CLL1 that is less than 20% of the level of CLL1 in a wild-type counterpart cell population.
328. The cell population of any of embodiments 285-218, wherein the cell population can differentiate into a cell type which expresses CLL1 at a level that is reduced with regard to the level of CLL1 expressed by the same differentiated cell type which is derived from a CLL1-wildtype hematopoietic stem or progenitor cell.
329. The cell population of any of embodiments 285-328, wherein the hematopoietic stem or progenitor cells are engineered such that a myeloid progenitor cell descended therefrom is deficient in CLL1 levels as compared with a myeloid progenitor cell descended from a CLL1-wildtype hematopoietic stem or progenitor cell.
330. The cell population of any of embodiments 285-328, wherein the hematopoietic stem or progenitor cells are engineered such that a myeloid cell (e.g., a terminally differentiated myeloid cell) descended therefrom is deficient in CLL1 levels as compared with a myeloid cell (e.g., a terminally differentiated myeloid cell) descended from a CLL1-wildtype hematopoietic stem or progenitor cell.
331. The cell population of any of embodiments 285-330, which further comprises one or more cells that comprise one or more non-engineered CLL1 genes.
332. The cell population of any of embodiments 285-331, which further comprises one or more cells that are homozygous wild-type for CLL1.
333. The cell population of any of embodiments 285-332, wherein about 0-1%, 1-2%, 2-5%, 5-10%, 10-15%, or 15-20% of cells in the population are homozygous wild-type for CLL1, e.g., are hematopoietic stem or progenitor cells that are homozygous wild-type for CLL1.
334. The cell population of any of embodiments 285-333 which further comprises one or more cells that are heterozygous for CLL1, e.g., comprise one wild-type copy of CLL1 and one mutant copy of CLL1.
335. The cell population of any of embodiments 285-334, wherein about 0-1%, 1-2%, 2-5%, 5-10%, 10-15%, or 15-20% of cells in the population are heterozygous wild-type for CLL1, e.g., are hematopoietic stem or progenitor cells that comprise one wild-type copy of CLL1 and one mutant copy of CLL1.
336. The cell population of any of embodiments 285-335, wherein at least 40%, 50%, 60%, 70%, 75%, 80%, 85%, 90%, or 95% of the copies of CLL1 in the population are mutant.
337. The cell population of any of embodiments 285-336, which comprises a plurality of different CLL1 mutations, e.g., which comprises at least 2, 3, 4, 5, 6, 7, 8, 9, or 10 different mutations.
338. The cell population of any of embodiments 285-337, which comprises at least 2, 3, 4, 5, 6, 7, 8, 9, or 10 different mutations.
339. The cell population of any of embodiments 285-338, which comprises at 2, 3, 4, 5, 6, 7, 8, 9, or 10 different insertions.
340. The cell population of any of embodiments 285-339, which comprises a plurality of insertions and a plurality of deletions.
341. The cell population of any of embodiments 285-340, which expresses less than 20% of the CLL1 expressed by a wild-type counterpart cell population.
342. The cell population of any of embodiments 285-341, wherein the reduced expression level of CLL1 is in a cell differentiated from (e.g., terminally differentiated from) the hematopoietic stem or progenitor cell, and the wild-type counterpart cell is a cell differentiated from (e.g., terminally differentiated from) a wild-type hematopoietic stem or progenitor cell.
343. The cell population of embodiment 342, wherein the cell differentiated from the hematopoietic stem or progenitor cell is a myeloblast, monocyte, or myeloid dendritic cell.
344. The cell population of any of embodiments 285-343, which, when administered to a subject, produces hCD45+ cells in the subject, e.g., when assayed at 16 weeks after administration.
345. The cell population of embodiment 344, which produces levels of hCD45+ cells comparable to the levels of hCD45+ cells produced with an otherwise similar cell population that is CLL1 wildtype.
346. The cell population of embodiments 344 or 345, which produces levels of hCD45+ cells that is at least 70%, 80%, 85%, 90%, or 95% the levels of hCD45+ cells produced by an otherwise similar cell population that is CLL1 wildtype.
347. The cell population of any of embodiments 285-346, which, when administered to a subject, produces hCD34+ cells in the subject, e.g., when assayed at 16 weeks after administration.
348. The cell population of embodiment 347, which produces levels of hCD34+ cells comparable to the levels of hCD34+ cells produced with an otherwise similar cell population that is CLL1 wildtype.
349. The cell population of embodiments 347 or 348, which produces levels of hCD34+ cells that is at least 70%, 80%, 85%, 90%, or 95% the levels of hCD34+ cells produced by an otherwise similar cell population that is CLL1 wildtype.
350. The cell population of any of embodiments 285-349, which, when administered to a subject, produces mast cells, basophils, eosinophils, common dendric cells (cDCs), plasmacytoid dendric cells (pDCs), neutrophils, monocytes, T cells, B, cells or any combination thereof, in the subject, e.g., when assayed at 16 weeks after administration.
351. The cell population of embodiment 350, which produces levels of mast cells, basophils, eosinophils, common dendric cells (cDCs), plasmacytoid dendric cells (pDCs), neutrophils, monocytes, T cells, B, cells or any combination thereof comparable to the levels of said cell type produced with an otherwise similar cell population that is CLL1 wildtype.
352. The cell population of embodiments 350 or 351, which produces levels of mast cells, basophils, eosinophils, common dendric cells (cDCs), plasmacytoid dendric cells (pDCs), neutrophils, monocytes, T cells, B, cells or any combination thereof that is at least 70%, 80%, 85%, 90%, or 95% the levels of said cell type produced by an otherwise similar cell population that is CLL1 wildtype.
353. The cell population of any of embodiments 350-352, wherein the produced cells are detected in a blood sample, a bone marrow sample, or a spleen sample obtained from the subject.
354. The cell population of any of embodiments 285-353, which, when administered to a subject, persists for at least 8, 12, or 16 weeks in the subject.
355. The cell population of any of embodiments 285-354, which, when administered to a subject, provides multilineage hematopoietic reconstitution.
356. The cell population of any of embodiments 285-355, which, produces CD14+ cells, e.g., when assayed at 7 or 14 days after genetic engineering.
357. The cell population of embodiment 356, which produces levels of CD14+ cells comparable to the levels of CD14+ cells produced with an otherwise similar cell population that is CLL1 wildtype.
358. The cell population of embodiments 356 or 357, which produces levels of CD14+ cells that is at least 70%, 80%, 85%, 90%, or 95% the levels of CD14+ cells produced by an otherwise similar cell population that is CLL1 wildtype.
359. The cell population of embodiments any of 356-358, wherein CD14+ levels are assayed after culturing in vitro in myeloid differentiation media.
360. The cell population of any of embodiments 285-359, which, produces CD11b+ cells, e.g., when assayed at 7 or 14 days after genetic engineering.
361. The cell population of embodiment 360, which produces levels of CD11b+ cells comparable to the levels of CD11b+ cells produced with an otherwise similar cell population that is CLL1 wildtype.
362. The cell population of embodiments 360 or 361, which produces levels of CD11b+ cells that is at least 70%, 80%, 85%, 90%, or 95% the levels of CD11b+ cells produced by an otherwise similar cell population that is CLL1 wildtype.
363. The cell population of embodiments any of 356-362, wherein CD15+ levels are assayed after culturing in vitro in myeloid differentiation media.
364. The cell population of any of embodiments 285-362, which, produces CD15+ cells, e.g., when assayed at 7 or 14 days after genetic engineering.
365. The cell population of embodiment 364, which produces levels of CD15+ cells comparable to the levels of CD11b+ cells produced with an otherwise similar cell population that is CLL1 wildtype.
366. The cell population of embodiments 364 or 365, which produces levels of CD15+ cells that is at least 70%, 80%, 85%, 90%, or 95% the levels of CD15+ cells produced by an otherwise similar cell population that is CLL1 wildtype.
367. The cell population of embodiments any of 356-366, wherein CD15+ levels are assayed after culturing in vitro in myeloid differentiation media.
368. The cell population of any of embodiments 285-367, wherein the most abundant mutation in CLL1 in the cell population is a deletion, e.g., a deletion of 1 nt, 2 nt, 3 nt, or 4 nt.
369. The cell population of any of embodiments 285-367, wherein the most abundant mutation in CLL1 in the cell population is an insertion, e.g., an insertion of 1 nt.
370. The cell population of any of embodiments 285-368, wherein the most abundant mutation in the cell population within the sequence of SEQ ID NO: 16 in CLL1 is a deletion, e.g., a deletion of 1 nt.
371. The cell population of embodiment 285-370, which further comprises one or both of a 2 nt deletion or a 1 nt insertion within the sequence of SEQ ID NO: 16 in a copy of CLL1.
372. The cell population of any of embodiments 285-368, wherein the most abundant mutation in the cell population within the sequence of SEQ ID NO: 14 in CLL1 is an insertion, e.g., an insertion of 1 nt.
373. The cell population of embodiment 285-368 or 224aa, which further comprises a 1 nt deletion within the sequence of SEQ ID NO: 14 in a copy of CLL1.
374. The cell population of any of embodiments 188-368, wherein the most abundant mutation in the cell population within the sequence of SEQ ID NO: 17 in CLL1 is a deletion, e.g., a deletion of 2 nt.
375. The cell population of embodiment 285-368 or 374, which further comprises a 1 nt insertion within the sequence of SEQ ID NO: 17 in a copy of CLL1.
376. The cell population of any of embodiments 285-368, wherein the most abundant mutation in the cell population within the sequence of SEQ ID NO: 17 in CLL1 is an insertion, e.g., an insertion of 1 nt.
377. The cell population of embodiment 285-368 or 376, which further comprises a 2 nt deletion within the sequence of SEQ ID NO: 40 in a copy of CLL1.
378. The cell population of any of embodiments 285-368, wherein the most abundant mutation in the cell population within the sequence of SEQ ID NO: 43 in CLL1 is a deletion, e.g., a deletion of 4 nt.
379. The cell population of embodiment 285-368 or 378, which further comprises a 1 nt insertion within the sequence of SEQ ID NO: 43 in a copy of CLL1.
380. The cell population of any of embodiments 285-379, which comprises hematopoietic stem cells and hematopoietic progenitor cells.
381. The cell population of any of embodiments 285-380, wherein at least 80%, 85%, 90%, 95%, or 98% of cells in the population are viable.
382. The cell population of any of embodiments 285-381, wherein at least 50%, 60%, 70%, 80%, or 90% of copies of CLL1 comprise a mutation.
383. The cell population of any of embodiments 285-382, wherein at least 50%, 60%, 65%, 70%, 75%, 80%, 85%, 90%, or 95% of cells in the population are negative for cell surface expression of CLL1, e.g., using a flow cytometry assay for CLL1 cell surface expression.
384. A pharmaceutical composition comprising the genetically engineered hematopoietic stem or progenitor cell of any of embodiments 184 or 186-284.
385. A pharmaceutical composition comprising the cell population of any of embodiments 285-383.
386. A mixture, e.g., a reaction mixture comprising:
a) a gRNA of any of embodiments 1-105, or gRNAs of a composition or kit of any of embodiments 106-133; and
b) a cell, e.g., a hematopoietic cell, e.g., an HSC or HPC, e.g., a genetically engineered cell of any of embodiments 184 or 186-284.
   387. The mixture of embodiment 386, wherein the cell is a wild-type cell or a cell having a mutation in CLL1.
   388. A kit comprising any two or more (e.g., three or all) of:
a) a gRNA of any of embodiments 1-105, or gRNAs of a composition or kit of any of embodiments 106-133;
b) a cell, e.g., a hematopoietic cell, e.g., an HSC or HPC, e.g., a genetically engineered cell of any of embodiments 184 or 186-284;
c) a Cas9 molecule; and
d) agent that targets CLL1, e.g., an agent as described herein.
   389. The kit of embodiment 388, which comprises (a) and (b), (a) and (c), (a) and d), (b) and (c), (b) and (d), or (c) and (d).
   390. A method of making the genetically engineered cell (e.g., hematopoietic stem or progenitor cell) of any of embodiments 184 or 186-284, or the cell population of any of embodiments 285-383, which comprises:

(i) providing a cell (e.g., a hematopoietic stem or progenitor cell, e.g., a wild-type hematopoietic stem or progenitor cell), and

(ii) introducing into the cell a nuclease (e.g., an endonuclease) that cleaves the target domain,

thereby producing a genetically engineered hematopoietic stem or progenitor cell.

391. The method of embodiment 390, wherein (ii) comprises introducing into the cell a gRNA that binds the target domain (e.g., a gRNA of any of embodiments 1-105 and an endonuclease that binds the gRNA).
392. The method of embodiment 390, wherein the endonuclease is a ZFN, TALEN, or meganuclease.
393. A method of supplying HSCs, HPCs, or HSPCs to a subject, comprising administering to the subject a plurality of cells of any of embodiments 184 or 186-284, or the cell population of any of embodiments 285-383.
394. A method, comprising administering to a subject a subject in need thereof a plurality of cells of any of embodiments 184 or 186-284, or the cell population of any of embodiments 285-383.
395. The method of embodiment 393 or 394, wherein the subject has a cancer, wherein cells of the cancer express CLL1 (e.g., wherein at least a plurality of the cancer cells express CLL1).
396. The method of any of embodiments 393-395, which further comprises administering to the subject an effective amount of an agent that targets CLL1, and wherein the agent comprises an antigen-binding fragment that binds CLL1.
397. The method of embodiment 396, wherein the agent that targets CLL1 is an immune cell expressing a chimeric antigen receptor (CAR), which comprises the antigen-binding fragment that binds CLL1.
398. A genetically engineered hematopoietic stem or progenitor cell of any of embodiments 184 or 186-284 or a cell population of any of embodiments 285-383 for use in treating a hematopoietic disorder, wherein the treating comprises administering to a subject in need thereof an effective amount of the genetically engineered hematopoietic stem or progenitor cell or the cell population, and further comprises administering to the subject an effective amount of an agent that targets CLL1, wherein the agent comprises an antigen-binding fragment that binds CLL1.
399. An agent that targets CLL1, wherein the agent comprises an antigen-binding fragment that binds CLL1, for use in treating a hematopoietic disorder, wherein the treating comprises administering to a subject in need thereof an effective amount of the agent that targets CLL1, and further comprises administering to the subject an effective amount of a genetically engineered hematopoietic stem or progenitor cell of any of embodiments 184 or 186-284 or a cell population of any of embodiments 285-383.
400. A combination of a genetically engineered hematopoietic stem or progenitor cell of any of embodiments 184 or 186-284 or a cell population of any of embodiments 285-383, and an agent that targets CLL1, wherein the agent comprises an antigen-binding fragment that binds CLL1, for use in treating a hematopoietic disorder, wherein the treating comprises administering to a subject in need thereof an effective amount of the genetically engineered hematopoietic stem or progenitor cell or the cell population, and the agent that binds CLL1.
401. A genetically engineered hematopoietic stem or progenitor cell of any of embodiments 184 or 186-284 or a cell population of any of embodiments 285-384 for use in cancer immunotherapy.
402. A genetically engineered hematopoietic stem or progenitor cell of any of embodiments 184 or 186-284 or a cell population of any of embodiments 285-384 for use in cancer immunotherapy, wherein the subject has a hematopoietic disorder.
403. A genetically engineered hematopoietic stem or progenitor cell of any of embodiments 184 or 186-284 or a cell population of any of embodiments 285-384 for use in hematopoietic repopulation of a subject having a hematopoietic disorder.
404. A genetically engineered hematopoietic stem or progenitor cell of any of embodiments 184 or 186-284 or a cell population of any of embodiments 285-384 for use in a method of treating a hematopoietic disorder, whereby the genetically engineered hematopoietic stem or progenitor cell described herein or a cell population described herein repopulate the subject.
405. A genetically engineered hematopoietic stem or progenitor cell of any of embodiments 184 or 186-284 or a cell population of any of embodiments 285-384 for use in reducing cytotoxic effects of an agent that targets CLL1 in immunotherapy.
406. A genetically engineered hematopoietic stem or progenitor cell of any of embodiments 184 or 186-284 or a cell population of any of embodiments 285-384 for use in an immunotherapy method using an agent that targets CLL1, whereby the genetically engineered hematopoietic stem or progenitor cell described herein or a cell population described herein reduces cytotoxic effects of the agent that targets CLL1.
407. The method, cell, agent, or combination of any of embodiments 285-384, wherein the genetically engineered hematopoietic stem or progenitor cell or the cell population is administered concomitantly with the agent that targets CLL1.
408. The method, cell, agent, or combination of any of embodiments 393-407, wherein the genetically engineered hematopoietic stem or progenitor cell or the cell population is administered prior to the agent that targets CLL1.
409. The method, cell, agent, or combination of any of embodiments 393-408, wherein the agent that targets CLL1 is administered prior to the genetically engineered hematopoietic stem or progenitor cell or the cell population.
410. The method, cell, agent, or combination of any of embodiments 393-409, wherein the immune cell is a T cell.
411. The method, cell, agent, or combination of any of embodiments 393-410, wherein the immune cell, the genetically engineered hematopoietic stem and/or progenitor cell, or both, are allogeneic.
412. The method, cell, agent, or combination of any of embodiments 393-411, wherein the immune cell, the genetically engineered hematopoietic stem and/or progenitor cell, or both, are autologous.
413. The method, cell, agent, or combination of any of embodiments 393-412, wherein the antigen-binding fragment in the chimeric receptor is a single-chain antibody fragment (scFv) that specifically binds human CLL1.
414. The method, cell, agent, or combination of any of embodiments 393-413, wherein hematopoietic disorder is a cancer, and wherein at least a plurality of cancer cells in the cancer express CLL1.
415. The method, cell, agent, or combination of any of embodiments 393-414, wherein the subject has a hematopoietic malignancy, e.g., a hematopoietic malignancy chosen from Hodgkin's lymphoma, non-Hodgkin's lymphoma, leukemia (e.g., acute myeloid leukemia, acute lymphoid leukemia, chronic myelogenous leukemia, acute lymphoblastic leukemia or chronic lymphoblastic leukemia, and chronic lymphoid leukemia), or multiple myeloma.
416. The method, cell, agent, or combination of any of embodiments 393-415, wherein the subject has a hematological disorder, e.g., a precancerous condition, e.g., a myelodysplasia, a myelodysplastic syndrome (MDS), or a preleukemia.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF THE DRAWINGS

FIG. 1 is a graph showing CLL1 gRNA screening on CD34⁺ cells. Human CD34⁺ cells were electroporated with Cas9 protein and CLL1-targeting gRNAs (listed on the y-axis). Editing efficiency of CLEC12A locus, shown on the x-axis, was determined by Sanger sequencing and TIDE analysis.

FIGS. 2A-2D are a series of diagrams showing survival and differentiation of CLL1-edited CD34⁺ cells. (A) Schematic showing the workflow of the experiment. Human CD34⁺ cells were electroporated with Cas9 protein and CLL1-targeting gRNA, followed by analysis of editing efficiency by TIDE and a CFU assay to assess in vitro differentiation. (B) Cell viability was measured 48 hours post electroporation. No Cas9 RNP group was used as control. (C) Editing efficiency of CLEC12A locus was determined by Sanger sequencing and TIDE analysis. (D) Control or CLL1-edited CD34⁺ cells were plated in Methocult 2 days after electroporation and scored for colony formation after 14 days. BFU-E: burst forming unit-erythroid; CFU-GM: colony forming unit-granulocyte/macrophage; CFU-GEMM: colony forming unit of multipotential myeloid progenitor cells (generate granulocytes, erythrocytes, monocytes, and megakaryocytes). Student's t-test was used.

FIG. 3 shows target expression on AML cell lines. The expression of CD33, CD123 and CLL1 in MOLM-13 and THP-1 cells and an unstained control was determined by flow cytometric analysis. The X-axis indicates the intensity of antibody staining and the Y-axis corresponds to number of cells.

FIG. 4 shows CD33- and CLL1-modified HL-60 cells. The expression of CD33 and CLL1 in wild-type (WT), CD33^{-/-}, CLL1^{-/-} and CD33^{-/-} CLL1^{-/-} HL-60 cells was assessed by flow cytometry. For the generation of CD33^{-/-} or CLL1^{-/-} HL-60 cells, WT HL-60 cells were electroporated with CD33- or CLL1-targeting RNP, followed by flow cytometric sorting of CD33- or CLL1-negative cells. CD33^{-/-}CLL1^{-/-} HL-60 cells were generated by electroporating CD33^{-/-} cells with CLL1-targeting RNP and sorted for CLL1-negative population. The X-axis indicates the intensity of antibody staining and the Y-axis corresponds to number of cells.

FIG. 5 shows an in vitro cytotoxicity assay of CD33 and CLL1 CAR-Ts. Anti-CD33 CAR-T and anti-CLL1 CAR-T were incubated with wild-type (WT), CD33^{-/-}, CLL1^{-/-} and CD33^{-/-} CLL1^{-/-} HL-60 cells, and cytotoxicity was assessed by flow cytometry. Non-transduced T cells were used as mock CAR-T control. The CARpool group was composed of 1:1 pooled combination of anti-CD33 and anti-CLL1 CAR-T cells. Student's t test was used. ns=not significant; *P<0.05; **P<0.01, ***P<0.001, ****P<0.0001. The Y-axis indicates the percentage of specific killing.

FIG. 6 shows gene-editing efficiency of CD34+ cells. Human CD34+ cells were electroporated with Cas9 protein and CD33-, CD123- or CLL1-targeting gRNAs, either alone or in combination. Editing efficiency of CD33, CD123 or CLL1 locus was determined by Sanger sequencing and TIDE analysis. The Y-axis indicates the editing efficiency (% by TIDE).

FIGS. 7A-7C shows in vitro colony formation of gene-edited CD34+ cells. Control or CD33, CD123, CLL-1-modified CD34+ cells were plated in Methocult 2 days after electroporation and scored for colony formation after 14 days. BFU-E: burst forming unit-erythroid; CFU-GM: colony forming unit-granulocyte/macrophage; CFU-GEMM: colony forming unit of multipotential myeloid progenitor cells (generate granulocytes, erythrocytes, monocytes, and megakaryocytes). Student's t test was used.

FIG. 8 shows gene editing frequency of CD34+ cells. Human CD34+ cells were electroporated with ribonucleoprotein (RNP) complexes composed of Cas9 protein and the CLL1-targeting gRNAs indicated on the X-axis, the sequences of which are found in Table 8. Editing frequency of the CLL1 locus was determined by Sanger sequencing. The Y-axis indicates the editing frequency.

FIG. 9 shows gene editing frequency of CD34+ cells. Human CD34+ cells were electroporated with Cas9 protein and the CLL1-targeting gRNAs indicated on the X-axis, specifically from left to right, gRNA D, F, G, O2, and P2. Editing frequency of the CLL1 locus was determined by Sanger sequencing. The Y-axis indicates the editing frequency. All gRNAs in FIG. 9 led to an editing frequency ≥80%.

FIG. 10 shows the INDEL (insertion/deletion) distribution for human CD34+ cells edited with the CLL1-targeting gRNAs, specifically gRNA D (top left), gRNA F (middle left), gRNA G (bottom left), gRNA 02 (top right), and gRNA P2 (middle right). The X-axis indicates the size of the INDEL and the Y-axis indicates the percentage of the specific INDEL in the mixture.

FIG. 11 is a schematic and overview of the protocol and experimental procedure/timeline used for in vivo characterization of CLL1-edited HSPCs in NBSGW mice.

FIGS. 12A-12D depict long-term lineage engraftment of CLL1-edited cells in the bone marrow of mice 16 weeks post-engraftment of non-edited control cells or CLL1KO cells. FIG. 12A shows the rates of human leukocyte chimerism calculated as percentage of human CD45+ (hCD45+) cells in the total CD45+ cell population (the sum of human and mouse CD45+ cells) in the bone marrow at week 16 post-engraftment of control cells (EP ctrl) or CLL1 KO cells edited with gRNA F. FIG. 12B shows the percentage of hCD45+ cells that were also positive for human CD34 (hCD34+) in the bone marrow at week 16 post-engraftment of control cells (EP ctrl) or CLL1KO cells edited with gRNA F. FIG. 12C shows the percentage of hCD45+ cells that were B-cells, T cells, monocytes, neutrophils, conventional dendritic cells (cDCs), plasmacytoid dendritic cells (pDCs), eosinophils, basophils, and mast cells) in the bone marrow at week 16 post-engraftment of control cells (EP ctrl) or CLL1KO cells edited with gRNA F. FIG. 12D shows the percentages of hCD45+ that were also CLL1+ quantified in the bone marrow at week 16 post-engraftment of control cells (EP ctrl) or CLL1KO cells edited with gRNA F.

FIG. 13A shows cell-surface expression of CLL1 in vitro as measured by FACs in, from top to bottom, non-edited control cells, CLL1 KO cells edited by gRNA F (editing frequency of 79.2% as measured by TIDE), and an FMO (fluorescence minus one) control. FIG. 13B shows the quantification granulocytes produced over time from in vitro culturing of non-edited control cells (EP ctrl) or CLL1 KO cells edited by gRNA F. FIG. 13C shows the quantification monocytes produced over time from in vitro culture of non-edited control cells (EP ctrl) or CLL1KO cells edited by gRNA F.

FIG. 14A shows the percentage of CLL1+ granulocytes (top) or monocytes (bottom) produced over time from in vitro culturing non-edited control cells (EP cntrl) or CLL1 KO cells edited by gRNA F. FIG. 14B shows the percentage of CD15+ (top left) or CD11b+ positive granulocytes (top right) or the percentage of CD14+ (bottom left) or CD11b+ positive monocytes (bottom right) quantified at day 0, 7, and 14 following editing and culture of non-edited control cells or CLL1KO cells edited by gRNA F.

FIG. 15 shows the percentage of phagocytosis measured in granulocytes (top) or monocytes (bottom) produced from non-edited control cells (EP ctrl) or CLL1 KO cells edited by the gRNA F.

FIG. 16A shows the production of IL-6 in pg/mL (right) or TNF-α in pg/mL (left) by granulocytes produced from non-edited control cells (EP ctrl) or CLL1 KO cells edited by gRNA F, that were unstimulated, stimulated by LPS, or stimulated by R848. FIG. 16B shows the production of IL-6 in pg/mL (right) or TNF-α in pg/mL (left) by monocytes produced from non-edited control cells (EP ctrl) or CLL1KO cells edited by gRNA F that were unstimulated, stimulated by LPS, or stimulated by R848.

FIG. 17A-17B shows in vitro colony formation of gene-edited CD34+ cells. Control or CLL1-modified CD34+ cells were plated in after electroporation and scored for colony formation after 14 days. BFU-E: burst forming unit-erythroid; CFU-GM: colony forming unit-granulocyte/macrophage; CFU-GEMM: colony forming unit of multipotential myeloid progenitor cells (generate granulocytes, erythrocytes, monocytes, and megakaryocytes). FIG. 17A shows colony count of BFU-E, CFU-G/M/GM, or CFU-GEMM that resulted from non-edited cells (EP ctrl) or CLL1KO cells edited by gRNA F (editing frequency of 79.2%). FIG. 17B shows percent colony distribution of BFU-E, CFU-G/M/GM, or CFU-GEMM that resulted from non-edited cells (EP ctrl) or CLL1KO cells edited by gRNA F.

### DETAILED DESCRIPTION OF THE INVENTION

### Definitions

The term “binds”, as used herein with reference to a gRNA interaction with a target domain, refers to the gRNA molecule and the target domain forming a complex. The complex may comprise two strands forming a duplex structure, or three or more strands forming a multi-stranded complex. The binding may constitute a step in a more extensive process, such as the cleavage of the target domain by a Cas endonuclease. In some embodiments, the gRNA binds to the target domain with perfect complementarity, and in other embodiments, the gRNA binds to the target domain with partial complementarity, e.g., with one or more mismatches. In some embodiments, when a gRNA binds to a target domain, the full targeting domain of the gRNA base pairs with the targeting domain. In other embodiments, only a portion of the target domain and/or only a portion of the targeting domain base pairs with the other. In an embodiment, the interaction is sufficient to mediate a target domain-mediated cleavage event.

A “Cas9 molecule” as that term is used herein, refers to a molecule or polypeptide that can interact with a gRNA and, in concert with the gRNA, home or localize to a site which comprises a target domain. Cas9 molecules include naturally occurring Cas9 molecules and engineered, altered, or modified Cas9 molecules that differ, e.g., by at least one amino acid residue, from a naturally occurring Cas9 molecule.

The terms “gRNA” and “guide RNA” are used interchangeably throughout and refer to a nucleic acid that promotes the specific targeting or homing of a gRNA/Cas9 molecule complex to a target nucleic acid. A gRNA can be unimolecular (having a single RNA molecule), sometimes referred to herein as sgRNAs, or modular (comprising more than one, and typically two, separate RNA molecules). A gRNA may bind to a target domain in the genome of a host cell. The gRNA may comprise a targeting domain that may be partially or completely complementary to the target domain. The gRNA may also comprise a “scaffold sequence,” (e.g., a tracrRNA sequence), that recruits a Cas9 molecule to a target domain bound to a gRNA sequence (e.g., by the targeting domain of the gRNA sequence). The scaffold sequence may comprise at least one stem loop structure and recruits an endonuclease. Exemplary scaffold sequences can be found, for example, in Jinek, et al. Science (2012) 337(6096):816-821, Ran, et al. Nature Protocols (2013) 8:2281-2308, PCT Application No. WO2014/093694, and PCT Application No. WO2013/176772.

The term “mutation” is used herein to refer to a genetic change (e.g., insertion, deletion, or substitution) in a nucleic acid compared to a reference sequence, e.g., the corresponding wild-type nucleic acid. In some embodiments, a mutation to a gene detargetizes the protein produced by the gene. In some embodiments, a detargetized CLL1 protein is not bound by, or is bound at a lower level by, an agent that targets CLL1.

The “targeting domain” of the gRNA is complementary to the “target domain” on the target nucleic acid. The strand of the target nucleic acid comprising the nucleotide sequence complementary to the core domain of the gRNA is referred to herein as the “complementary strand” of the target nucleic acid. Guidance on the selection of targeting domains can be found, e.g., in Fu Y et al, Nat Biotechnol 2014 (doi: 10.1038/nbt.2808) and Sternberg S H et al., Nature 2014 (doi: 10.1038/nature13011).

### Nucleases

In some embodiments, a cell (e.g., HSC or HPC) described herein is made using a nuclease described herein. Exemplary nucleases include Cas molecules (e.g., Cas9, TALENs, ZFNs, and meganucleases. In some embodiments, a nuclease is used in combination with a CLL1 gRNA described herein (e.g., according to Table 2, 6, or 8).

Cas9 Molecules

In some embodiments, a CLL1 gRNA described herein is complexed with a Cas9 molecule. Various Cas9 molecules can be used. In some embodiments, a Cas9 molecule is selected that has the desired PAM specificity to target the gRNA/Cas9 molecule complex to the target domain in CLL1. In some embodiments, genetically engineering a cell also comprises introducing one or more (e.g., 1, 2, 3 or more) Cas9 molecules into the cell.

Cas9 molecules of a variety of species can be used in the methods and compositions described herein. In embodiments, the Cas9 molecule is of, or derived from, S. pyogenes (SpCas9), S. aureus (SaCas9) or S. thermophilus. Additional suitable Cas9 molecules include those of, or derived from, Staphylococcus aureus, Neisseria meningitidis (NmCas9), Acidovorax avenae, Actinobacillus pleuropneumoniae, Actinobacillus succinogenes, Actinobacillus suis, Actinomyces sp., cycliphilus denitrificans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacter jejuni (CjCas9), Campylobacter lari, Candidatus Puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, gamma proteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria meningitidis, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum sp., Tistrella mobilis, Treponema sp., or Verminephrobacter eiseniae.

In some embodiments, the Cas9 molecule is a naturally occurring Cas9 molecule. In some embodiments, the Cas9 molecule is an engineered, altered, or modified Cas9 molecule that differs, e.g., by at least one amino acid residue, from a reference sequence, e.g., the most similar naturally occurring Cas9 molecule or a sequence of Table 50 of WO2015157070, which is herein incorporated by reference in its entirety. In some embodiments, the Cas9 molecule comprises Cpf1 or a fragment or variant thereof.

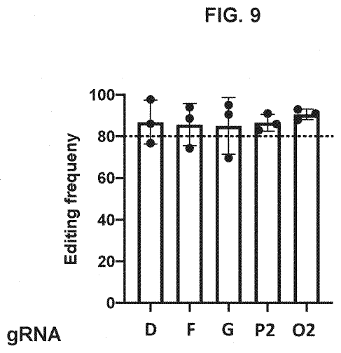

A naturally occurring Cas9 molecule typically comprises two lobes: a recognition (REC) lobe and a nuclease (NUC) lobe; each of which further comprises domains described, e.g., in WO2015157070, e.g., in FIGS. 9A-9B therein (which application is incorporated herein by reference in its entirety).

The REC lobe comprises the arginine-rich bridge helix (BH), the REC1 domain, and the REC2 domain. The REC lobe appears to be a Cas9-specific functional domain. The BH domain is a long alpha helix and arginine rich region and comprises amino acids 60-93 of the sequence of S. pyogenes Cas9. The REC1 domain is involved in recognition of the repeat:anti-repeat duplex, e.g., of a gRNA or a tracrRNA. The REC1 domain comprises two REC1 motifs at amino acids 94 to 179 and 308 to 717 of the sequence of S. pyogenes Cas9. These two REC1 domains, though separated by the REC2 domain in the linear primary structure, assemble in the tertiary structure to form the REC1 domain. The REC2 domain, or parts thereof, may also play a role in the recognition of the repeat: anti-repeat duplex. The REC2 domain comprises amino acids 180-307 of the sequence of S. pyogenes Cas9.

The NUC lobe comprises the RuvC domain (also referred to herein as RuvC-like domain), the HNH domain (also referred to herein as HNH-like domain), and the PAM-interacting (PI) domain. The RuvC domain shares structural similarity to retroviral integrase superfamily members and cleaves a single strand, e.g., the non-complementary strand of the target nucleic acid molecule. The RuvC domain is assembled from the three split RuvC motifs (RuvC I, RuvCII, and RuvCIII, which are often commonly referred to in the art as RuvCI domain, or N-terminal RuvC domain, RuvCII domain, and RuvCIII domain) at amino acids 1-59, 718-769, and 909-1098, respectively, of the sequence of S. pyogenes Cas9. Similar to the REC1 domain, the three RuvC motifs are linearly separated by other domains in the primary structure, however in the tertiary structure, the three RuvC motifs assemble and form the RuvC domain. The HNH domain shares structural similarity with HNH endonucleases, and cleaves a single strand, e.g., the complementary strand of the target nucleic acid molecule. The HNH domain lies between the RuvC II-III motifs and comprises amino acids 775-908 of the sequence of S. pyogenes Cas9. The PI domain interacts with the PAM of the target nucleic acid molecule, and comprises amino acids 1099-1368 of the sequence of S. pyogenes Cas9.

Crystal structures have been determined for naturally occurring bacterial Cas9 molecules (Jinek et al., Science, 343(6176): 1247997, 2014) and for S. pyogenes Cas9 with a guide RNA (e.g., a synthetic fusion of crRNA and tracrRNA) (Nishimasu et al., Cell, 156:935-949, 2014; and Anders et al., Nature, 2014, doi: 10.1038/nature13579).

In some embodiments, a Cas9 molecule described herein has nuclease activity, e.g., double strand break activity. In some embodiments, the Cas9 molecule has been modified to inactivate one of the catalytic residues of the endonuclease. In some embodiments, the Cas9 molecule is a nickase and produces a single stranded break. See, e.g., Dabrowska et al. Frontiers in Neuroscience (2018) 12(75). It has been shown that one or more mutations in the RuvC and HNH catalytic domains of the enzyme may improve Cas9 efficiency. See, e.g., Sarai et al. Currently Pharma. Biotechnol. (2017) 18(13). In some embodiments, the Cas9 molecule is fused to a second domain, e.g., a domain that modifies DNA or chromatin, e.g., a deaminase or demethylase domain. In some such embodiments, the Cas9 molecule is modified to eliminate its endonuclease activity.

In some embodiments, a Cas9 molecule described herein is administered together with a template for homology directed repair (HDR). In some embodiments, a Cas9 molecule described herein is administered without a HDR template.

In some embodiments, the Cas9 molecule is modified to enhance specificity of the enzyme (e.g., reduce off-target effects, maintain robust on-target cleavage). In some embodiments, the Cas9 molecule is an enhanced specificity Cas9 variant (e.g., eSPCas9). See, e.g., Slaymaker et al. Science (2016) 351 (6268): 84-88. In some embodiments, the Cas9 molecule is a high fidelity Cas9 variant (e.g., SpCas9-HF1). See, e.g., Kleinstiver et al. Nature (2016) 529: 490-495.

Various Cas9 molecules are known in the art and may be obtained from various sources and/or engineered/modified to modulate one or more activities or specificities of the enzymes. In some embodiments, the Cas9 molecule has been engineered/modified to recognize one or more PAM sequence. In some embodiments, the Cas9 molecule has been engineered/modified to recognize one or more PAM sequence that is different than the PAM sequence the Cas9 molecule recognizes without engineering/modification. In some embodiments, the Cas9 molecule has been engineered/modified to reduce off-target activity of the enzyme.

In some embodiments, the nucleotide sequence encoding the Cas9 molecule is modified further to alter the specificity of the endonuclease activity (e.g., reduce off-target cleavage, decrease the endonuclease activity or lifetime in cells, increase homology-directed recombination and reduce non-homologous end joining). See, e.g., Komor et al. Cell (2017) 168: 20-36. In some embodiments, the nucleotide sequence encoding the Cas9 molecule is modified to alter the PAM recognition of the endonuclease. For example, the Cas9 molecule SpCas9 recognizes PAM sequence NGG, whereas relaxed variants of the SpCas9 comprising one or more modifications of the endonuclease (e.g., VQR SpCas9, EQR SpCas9, VRER SpCas9) may recognize the PAM sequences NGA, NGAG, NGCG. PAM recognition of a modified Cas9 molecule is considered “relaxed” if the Cas9 molecule recognizes more potential PAM sequences as compared to the Cas9 molecule that has not been modified. For example, the Cas9 molecule SaCas9 recognizes PAM sequence NNGRRT, whereas a relaxed variant of the SaCas9 comprising one or more modifications (e.g., KKH SaCas9) may recognize the PAM sequence NNNRRT. In one example, the Cas9 molecule FnCas9 recognizes PAM sequence NNG, whereas a relaxed variant of the FnCas9 comprising one or more modifications of the endonuclease (e.g., RHA FnCas9) may recognize the PAM sequence YG. In one example, the Cas9 molecule is a Cpf1 endonuclease comprising substitution mutations S542R and K607R and recognize the PAM sequence TYCV. In one example, the Cas9 molecule is a Cpf1 endonuclease comprising substitution mutations S542R, K607R, and N552R and recognize the PAM sequence TATV. See, e.g., Gao et al. Nat. Biotechnol. (2017) 35(8): 789-792.

In some embodiments, more than one (e.g., 2, 3, or more) Cas9 molecules are used. In some embodiments, at least one of the Cas9 molecule is a Cas9 enzyme. In some embodiments, at least one of the Cas molecules is a Cpf1 enzyme. In some embodiments, at least one of the Cas9 molecule is derived from Streptococcus pyogenes. In some embodiments, at least one of the Cas9 molecule is derived from Streptococcus pyogenes and at least one Cas9 molecule is derived from an organism that is not Streptococcus pyogenes. In some embodiments, the Cas9 molecule is a base editor. Base editor endonuclease generally comprises a catalytically inactive Cas9 molecule fused to a function domain. See, e.g., Eid et al. Biochem. J. (2018) 475(11): 1955-1964; Rees et al. Nature Reviews Genetics (2018) 19:770-788. In some embodiments, the catalytically inactive Cas9 molecule is dCas9. In some embodiments, the endonuclease comprises a dCas9 fused to one or more uracil glycosylase inhibitor (UGI) domains. In some embodiments, the endonuclease comprises a dCas9 fused to an adenine base editor (ABE), for example an ABE evolved from the RNA adenine deaminase TadA. In some embodiments, the endonuclease comprises a dCas9 fused to cytidine deaminase enzyme (e.g., APOBEC deaminase, pmCDA1, activation-induced cytidine deaminase (AID)). In some embodiments, the catalytically inactive Cas9 molecule has reduced activity and is nCas9. In some embodiments, the catalytically inactive Cas9 molecule (dCas9) is fused to one or more uracil glycosylase inhibitor (UGI) domains. In some embodiments, the Cas9 molecule comprises a nCas9 fused to an adenine base editor (ABE), for example an ABE evolved from the RNA adenine deaminase TadA. In some embodiments, the Cas9 molecule comprises a nCas9 fused to cytidine deaminase enzyme (e.g., APOBEC deaminase, pmCDA1, activation-induced cytidine deaminase (AID)).

Examples of base editors include, without limitation, BE1, BE2, BE3, HF-BE3, BE4, BE4max, BE4-Gam, YE1-BE3, EE-BE3, YE2-BE3, YEE-CE3, VQR-BE3, VRER-BE3, SaBE3, SaBE4, SaBE4-Gam, Sa(KKH)-BE3, Target-AID, Target-AID-NG, xBE3, eA3A-BE3, BE-PLUS, TAM, CRISPR-X, ABE7.9, ABE7.10, ABE7.10*, xABE, ABESa, VQR-ABE, VRER-ABE, Sa(KKH)-ABE, and CRISPR-SKIP. Additional examples of base editors can be found, for example, in US Publication No. 2018/0312825A1, US Publication No. 2018/0312828A1, and PCT Publication No. WO 2018/165629A1, which are incorporated by reference herein in their entireties.

In some embodiments, the base editor has been further modified to inhibit base excision repair at the target site and induce cellular mismatch repair. Any of the Cas9 molecules described herein may be fused to a Gam domain (bacteriophage Mu protein) to protect the Cas9 molecule from degradation and exonuclease activity. See, e.g., Eid et al. Biochem. J. (2018) 475(11): 1955-1964.

In some embodiments, the Cas9 molecule belongs to class 2 type V of Cas endonuclease. Class 2 type V Cas endonucleases can be further categorized as type V-A, type V-B, type V-C, and type V-U. See, e.g., Stella et al. Nature Structural & Molecular Biology (2017). In some embodiments, the Cas molecule is a type V-A Cas endonuclease, such as a Cpf1 nuclease. In some embodiments, the Ca Cas9 molecule is a type V-B Cas endonuclease, such as a C2c1 endonuclease. See, e.g., Shmakov et al. Mol Cell (2015) 60: 385-397. In some embodiments, the Cas molecule is Mad7. Alternatively or in addition, the Cas9 molecule is a Cpf1 nuclease or a variant thereof. As will be appreciated by one of skill in the art, the Cpf1 nuclease may also be referred to as Cas12a. See, e.g., Strohkendl et al. Mol. Cell (2018) 71: 1-9. In some embodiments, a composition or method described herein involves, or a host cell expresses a Cpf1 nuclease derived from Provetella spp. or Francisella spp., Acidaminococcus sp. (AsCpf1), Lachnospiraceae bacterium (LpCpf1), or Eubacterium rectale. In some embodiments, the nucleotide sequence encoding the Cpf1 nuclease may be codon optimized for expression in a host cell. In some embodiments, the nucleotide sequence encoding the Cpf1 endonuclease is further modified to alter the activity of the protein.

In some embodiments, catalytically inactive variants of Cas molecules (e.g., of Cas9 or Cas12a) are used according to the methods described herein. A catalytically inactive variant of Cpf1 (Cas12a) may be referred to dCas12a. As described herein, catalytically inactive variants of Cpf1 maybe fused to a function domain to form a base editor. See, e.g., Rees et al. Nature Reviews Genetics (2018) 19:770-788. In some embodiments, the catalytically inactive Cas9 molecule is dCas9. In some embodiments, the endonuclease comprises a dCas12a fused to one or more uracil glycosylase inhibitor (UGI) domains. In some embodiments, the Cas9 molecule comprises a dCas12a fused to an adenine base editor (ABE), for example an ABE evolved from the RNA adenine deaminase TadA. In some embodiments, the Cas molecule comprises a dCas12a fused to cytidine deaminase enzyme (e.g., APOBEC deaminase, pmCDA1, activation-induced cytidine deaminase (AID)).

Alternatively or in addition, the Cas9 molecule may be a Cas14 endonuclease or variant thereof. Cas14 endonucleases are derived from archaea and tend to be smaller in size (e.g., 400-700 amino acids). Additionally Cas14 endonucleases do not require a PAM sequence. See, e.g., Harrington et al. Science (2018).

Any of the Cas9 molecules described herein may be modulated to regulate levels of expression and/or activity of the Cas9 molecule at a desired time. For example, it may be advantageous to increase levels of expression and/or activity of the Cas9 molecule during particular phase(s) of the cell cycle. It has been demonstrated that levels of homology-directed repair are reduced during the G1 phase of the cell cycle, therefore increasing levels of expression and/or activity of the Cas9 molecule during the S phase, G2 phase, and/or M phase may increase homology-directed repair following the Cas endonuclease editing. In some embodiments, levels of expression and/or activity of the Cas9 molecule are increased during the S phase, G2 phase, and/or M phase of the cell cycle. In one example, the Cas9 molecule fused to the N-terminal region of human Geminin. See, e.g., Gutschner et al. Cell Rep. (2016) 14(6): 1555-1566. In some embodiments, levels of expression and/or activity of the Cas9 molecule are reduced during the G1 phase. In one example, the Cas9 molecule is modified such that it has reduced activity during the G1 phase. See, e.g., Lomova et al. Stem Cells (2018).

Alternatively or in addition, any of the Cas9 molecules described herein may be fused to an epigenetic modifier (e.g., a chromatin-modifying enzyme, e.g., DNA methylase, histone deacetylase). See, e.g., Kungulovski et al. Trends Genet. (2016) 32(2):101-113. Cas9 molecule fused to an epigenetic modifier may be referred to as “epieffectors” and may allow for temporal and/or transient endonuclease activity. In some embodiments, the Cas9 molecule is a dCas9 fused to a chromatin-modifying enzyme.

Zinc Finger Nucleases

In some embodiments, a cell or cell population described herein is produced using zinc finger (ZFN) technology. In some embodiments, the ZFN recognizes a target domain described herein, e.g., in Table 1. In general, zinc finger mediated genomic editing involves use of a zinc finger nuclease, which typically comprises a zinc finger DNA binding domain and a nuclease domain. The zinc finger binding domain may be engineered to recognize and bind to any target domain of interest, e.g., may be designed to recognize a DNA sequence ranging from about 3 nucleotides to about 21 nucleotides in length, or from about 8 to about 19 nucleotides in length. Zinc finger binding domains typically comprise at least three zinc finger recognition regions (e.g., zinc fingers).

Restriction endonucleases (restriction enzymes) capable of sequence-specific binding to DNA (at a recognition site) and cleaving DNA at or near the site of binding are known in the art and may be used to form ZFN for use in genomic editing. For example, Type IIS restriction endonucleases cleave DNA at sites removed from the recognition site and have separable binding and cleavage domains. In one example, the DNA cleavage domain may be derived from the FokI endonuclease.

TALENs

In some embodiments, a cell or cell population described herein is produced using TALEN technology. In some embodiments, the TALEN recognizes a target domain described herein, e.g., in Table 1. In general, TALENs are engineered restriction enzymes that can specifically bind and cleave a desired target DNA molecule. A TALEN typically contains a Transcriptional Activator-Like Effector (TALE) DNA-binding domain fused to a DNA cleavage domain. The DNA binding domain may contain a highly conserved 33-34 amino acid sequence with a divergent 2 amino acid RVD (repeat variable dipeptide motif) at positions 12 and 13. The RVD motif determines binding specificity to a nucleic acid sequence and can be engineered to specifically bind a desired DNA sequence. In one example, the DNA cleavage domain may be derived from the FokI endonuclease. In some embodiments, the FokI domain functions as a dimer, using two constructs with unique DNA binding domains for sites in the target genome with proper orientation and spacing.

A TALEN specific to a target gene of interest can be used inside a cell to produce a double-stranded break (DSB). A mutation can be introduced at the break site if the repair mechanisms improperly repair the break via non-homologous end joining. For example, improper repair may introduce a frame shift mutation. Alternatively, a foreign DNA molecule having a desired sequence can be introduced into the cell along with the TALEN. Depending on the sequence of the foreign DNA and chromosomal sequence, this process can be used to correct a defect or introduce a DNA fragment into a target gene of interest, or introduce such a defect into the endogenous gene, thus decreasing expression of the target gene.

Some exemplary, non-limiting embodiments of endonucleases and nuclease variants suitable for use in connection with the guide RNAs and genetic engineering methods provided herein have been described above. Additional suitable nucleases and nuclease variants will be apparent to those of skill in the art based on the present disclosure and the knowledge in the art. The disclosure is not limited in this respect.

gRNA Sequences and Configurations

gRNA Configuration Generally

A gRNA can comprise a number of domains. In an embodiment, a unimolecular, sgRNA, or chimeric, gRNA comprises, e.g., from 5′ to 3′:
a targeting domain (which is complementary, or partially complementary, to a target nucleic acid sequence in a target gene, e.g., in the CLL1 gene;
a first complementarity domain;
a linking domain;
a second complementarity domain (which is complementary to the first complementarity domain);
a proximal domain; and
optionally, a tail domain.

Each of these domains is now described in more detail.

The targeting domain may comprise a nucleotide sequence that is complementary, e.g., at least 80, 85, 90, or 95% complementary, e.g., fully complementary, to the target sequence on the target nucleic acid. The targeting domain is part of an RNA molecule and will therefore typically comprise the base uracil (U), while any DNA encoding the gRNA molecule will comprise the base thymine (T). While not wishing to be bound by theory, in an embodiment, it is believed that the complementarity of the targeting domain with the target sequence contributes to specificity of the interaction of the gRNA/Cas9 molecule complex with a target nucleic acid. It is understood that in a targeting domain and target sequence pair, the uracil bases in the targeting domain will pair with the adenine bases in the target sequence. In an embodiment, the target domain itself comprises in the 5′ to 3′ direction, an optional secondary domain, and a core domain. In an embodiment, the core domain is fully complementary with the target sequence. In an embodiment, the targeting domain is 5 to 50 nucleotides in length. The targeting domain may be between 15-25 nucleotides, 18-22 nucleotides, or 19-21 nucleotides in length. In some embodiments, the targeting domain is 15, 16, 17, 18, 19, 20, 21, 22, 23, 24, or 25 nucleotides in length. In some embodiments, the targeting domain is between 10-30, or between 15-25, nucleotides in length.

In some embodiments, a targeting domain comprises a core domain and a secondary targeting domain, e.g., as described in International Application WO2015157070, which is incorporated by reference in its entirety. In an embodiment, the core domain comprises about 8 to about 13 nucleotides from the 3′ end of the targeting domain (e.g., the most 3′ 8 to 13 nucleotides of the targeting domain). In an embodiment, the secondary domain is positioned 5′ to the core domain. In many embodiments, the core domain has exact complementarity with the corresponding region of the target sequence. In other embodiments, the core domain can have 1 or more nucleotides that are not complementary with the corresponding nucleotide of the target sequence.

The first complementarity domain is complementary with the second complementarity domain, and in an embodiment, has sufficient complementarity to the second complementarity domain to form a duplexed region under at least some physiological conditions. In an embodiment, the first complementarity domain is 5 to 30 nucleotides in length. In an embodiment, the first complementarity domain comprises 3 subdomains, which, in the 5′ to 3′ direction are: a 5′ subdomain, a central subdomain, and a 3′ subdomain. In an embodiment, the 5′ subdomain is 4 to 9, e.g., 4, 5, 6, 7, 8 or 9 nucleotides in length. In an embodiment, the central subdomain is 1, 2, or 3, e.g., 1, nucleotide in length. In an embodiment, the 3′ subdomain is 3 to 25, e.g., 4 to 22, 4 to 18, or 4 to 10, or 3, 4, 5, 6, 7, 8, 9, 10, 11, 12, 13, 14, 15, 16, 17, 18, 19, 20, 21, 22, 23, 24, or 25 nucleotides in length. The first complementarity domain can share homology with, or be derived from, a naturally occurring first complementarity domain. In an embodiment, it has at least 50% homology with a S. pyogenes, S. aureus or S. thermophilus, first complementarity domain.

The sequence and placement of the above-mentioned domains are described in more detail in WO2015157070, which is herein incorporated by reference in its entirety, including p. 88-112 therein.

A linking domain serves to link the first complementarity domain with the second complementarity domain of a unimolecular gRNA. The linking domain can link the first and second complementarity domains covalently or non-covalently. In an embodiment, the linkage is covalent. In an embodiment, the linking domain is, or comprises, a covalent bond interposed between the first complementarity domain and the second complementarity domain. In some embodiments, the linking domain comprises one or more, e.g., 2, 3, 4, 5, 6, 7, 8, 9, or 10 nucleotides. In some embodiments, the linking domain comprises at least one non-nucleotide bond, e.g., as disclosed in WO2018126176, the entire contents of which are incorporated herein by reference.

The second complementarity domain is complementary, at least in part, with the first complementarity domain, and in an embodiment, has sufficient complementarity to the second complementarity domain to form a duplexed region under at least some physiological conditions. In an embodiment, the second complementarity domain can include a sequence that lacks complementarity with the first complementarity domain, e.g., a sequence that loops out from the duplexed region. In an embodiment, the second complementarity domain is 5 to 27 nucleotides in length. In an embodiment, the second complementarity domain is longer than the first complementarity region. In an embodiment, the complementary domain is 5, 6, 7, 8, 9, 10, 11, 12, 13, 14, 15, 16, 17, 18, 19, 20, 21, 22, 23, 24 or 25 nucleotides in length. In an embodiment, the second complementarity domain comprises 3 subdomains, which, in the 5′ to 3′ direction are: a 5′ subdomain, a central subdomain, and a 3′ subdomain. In an embodiment, the 5′ subdomain is 3 to 25, e.g., 4 to 22, 4 to 18, or 4 to 10, or 3, 4, 5, 6, 7, 8, 9, 10, 11, 12, 13, 14, 15, 16, 17, 18, 19, 20, 21, 22, 23, 24, or 25 nucleotides in length. In an embodiment, the central subdomain is 1, 2, 3, 4 or 5, e.g., 3, nucleotides in length. In an embodiment, the 3′ subdomain is 4 to 9, e.g., 4, 5, 6, 7, 8 or 9 nucleotides in length. In an embodiment, the 5′ subdomain and the 3′ subdomain of the first complementarity domain, are respectively, complementary, e.g., fully complementary, with the 3′ subdomain and the 5′ subdomain of the second complementarity domain.

In an embodiment, the proximal domain is 5 to 20 nucleotides in length. In an embodiment, the proximal domain can share homology with or be derived from a naturally occurring proximal domain. In an embodiment, it has at least 50% homology with an S. pyogenes, S. aureus or S. thermophilus, proximal domain.

A broad spectrum of tail domains are suitable for use in gRNAs. In an embodiment, the tail domain is 0 (absent), 1, 2, 3, 4, 5, 6, 7, 8, 9, or 10 nucleotides in length. In some embodiments, the tail domain nucleotides are from or share homology with a sequence from the 5′ end of a naturally occurring tail domain. In an embodiment, the tail domain includes sequences that are complementary to each other and which, under at least some physiological conditions, form a duplexed region. In an embodiment, the tail domain is absent or is 1 to 50 nucleotides in length. In an embodiment, the tail domain can share homology with or be derived from a naturally occurring proximal tail domain. In an embodiment, it has at least 50% homology with an S. pyogenes, S. aureus or S. thermophilus, tail domain. In an embodiment, the tail domain includes nucleotides at the 3′ end that are related to the method of in vitro or in vivo transcription.

In some embodiments, modular gRNA comprises:
a first strand comprising, e.g., from 5′ to 3′:
   a targeting domain (which is complementary to a target nucleic acid in the CLL1 gene) and
   a first complementarity domain; and
a second strand, comprising, preferably from 5′ to 3′:
   optionally, a 5′ extension domain;
   a second complementarity domain;
   a proximal domain; and
   optionally, a tail domain.

In some embodiments, the gRNA is chemically modified. For instance, the gRNA may comprise one or more modification chosen from phosphorothioate backbone modification, 2′-O-Me-modified sugars (e.g., at one or both of the 3′ and 5′ termini), 2′F-modified sugar, replacement of the ribose sugar with the bicyclic nucleotide-cEt, 3′thioPACE (MSP), or any combination thereof. Suitable gRNA modifications are described, e.g., in Randar et al. PNAS Dec. 22, 2015 112 (51) E7110-E7117 and Hendel et al., Nat Biotechnol. 2015 September; 33(9): 985-989, each of which is incorporated herein by reference in its entirety. In some embodiments, a gRNA described herein comprises one or more 2′-O-methyl-3′-phosphorothioate nucleotides, e.g., at least 2, 3, 4, 5, or 6 2′-O-methyl-3′-phosphorothioate nucleotides. In some embodiments, a gRNA described herein comprises modified nucleotides (e.g., 2′-O-methyl-3′-phosphorothioate nucleotides) at the three terminal positions and the 5′ end and/or at the three terminal positions and the 3′ end. In some embodiments, the gRNA may comprise one or more modified nucleotides, e.g., as described in International Applications WO/2017/214460, WO/2016/089433, and WO/2016/164356, which are incorporated by reference their entirety.

In some embodiments, a gRNA described herein is chemically modified. For example, the gRNA may comprise one or more 2′-O modified nucleotides, e.g., 2′-O-methyl nucleotides. In some embodiments, the gRNA comprises a 2′-O modified nucleotide, e.g., 2′-O-methyl nucleotide at the 5′ end of the gRNA. In some embodiments, the gRNA comprises a 2′-O modified nucleotide, e.g., 2′-O-methyl nucleotide at the 3′ end of the gRNA. In some embodiments, the gRNA comprises a 2′-O-modified nucleotide, e.g., 2′-O-methyl nucleotide at both the 5′ and 3′ ends of the gRNA. In some embodiments, the gRNA is 2′-O-modified, e.g. 2′-O-methyl-modified at the nucleotide at the 5′ end of the gRNA, the second nucleotide from the 5′ end of the gRNA, and the third nucleotide from the 5′ end of the gRNA. In some embodiments, the gRNA is 2′-O-modified, e.g. 2′-O-methyl-modified at the nucleotide at the 3′ end of the gRNA, the second nucleotide from the 3′ end of the gRNA, and the third nucleotide from the 3′ end of the gRNA. In some embodiments, the gRNA is 2′-O-modified, e.g. 2′-O-methyl-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified, e.g. 2′-O-methyl-modified at the second nucleotide from the 3′ end of the gRNA, the third nucleotide from the 3′ end of the gRNA, and at the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified, e.g. 2′-O-methyl-modified, at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA. In some embodiments, the 2′-O-methyl nucleotide comprises a phosphate linkage to an adjacent nucleotide. In some embodiments, the 2′-O-methyl nucleotide comprises a phosphorothioate linkage to an adjacent nucleotide. In some embodiments, the 2′-O-methyl nucleotide comprises a thioPACE linkage to an adjacent nucleotide.

In some embodiments, the gRNA may comprise one or more 2′-O-modified and 3′phosphorous-modified nucleotide, e.g., a 2′-O-methyl 3′phosphorothioate nucleotide. In some embodiments, the gRNA comprises a 2′-O-modified and 3′phosphorous-modified, e.g., 2′-O-methyl 3′phosphorothioate nucleotide at the 5′ end of the gRNA. In some embodiments, the gRNA comprises a 2′-O-modified and 3′phosphorous-modified, e.g., 2′-O-methyl 3′phosphorothioate nucleotide at the 3′ end of the gRNA. In some embodiments, the gRNA comprises a 2′-O-modified and 3′phosphorous-modified, e.g., 2′-O-methyl 3′phosphorothioate nucleotide at the 5′ and 3′ ends of the gRNA. In some embodiments, the gRNA comprises a backbone in which one or more non-bridging oxygen atoms has been replaced with a sulfur atom. In some embodiments, the gRNA is 2′-O-modified and 3′phosphorous-modified, e.g. 2′-O-methyl 3′phosphorothioate-modified at the nucleotide at the 5′ end of the gRNA, the second nucleotide from the 5′ end of the gRNA, and the third nucleotide from the 5′ end of the gRNA. In some embodiments, the gRNA is 2′-O-modified and 3′phosphorous-modified, e.g. 2′-O-methyl 3′phosphorothioate-modified at the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′phosphorothioate-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′phosphorothioate-modified at the second nucleotide from the 3′ end of the gRNA, the third nucleotide from the 3′ end of the gRNA, and the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified and 3′phosphorous-modified, e.g. 2′-O-methyl 3′phosphorothioate-modified at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA.

In some embodiments, the gRNA may comprise one or more 2′-O-modified and 3′-phosphorous-modified, e.g., 2′-O-methyl 3′thioPACE nucleotide. In some embodiments, the gRNA comprises a 2′-O-modified and 3′phosphorous-modified, e.g., 2′-O-methyl 3′thioPACE nucleotide at the 5′ end of the gRNA. In some embodiments, the gRNA comprises a 2′-O-modified and 3′phosphorous-modified, e.g., 2′-O-methyl 3′thioPACE nucleotide at the 3′ end of the gRNA. In some embodiments, the gRNA comprises a 2′-O-modified and 3′phosphorous-modified, e.g., 2′-O-methyl 3′thioPACE nucleotide at the 5′ and 3′ ends of the gRNA. In some embodiments, the gRNA comprises a backbone in which one or more non-bridging oxygen atoms have been replaced with a sulfur atom and one or more non-bridging oxygen atoms have been replaced with an acetate group. In some embodiments, the gRNA is 2′-O-modified and 3′phosphorous-modified, e.g. 2′-O-methyl 3′ thioPACE-modified at the nucleotide at the 5′ end of the gRNA, the second nucleotide from the 5′ end of the gRNA, and the third nucleotide from the 5′ end of the gRNA. In some embodiments, the gRNA is 2′-O-modified and 3′phosphorous-modified, e.g. 2′-O-methyl 3′thioPACE-modified at the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′thioPACE-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′thioPACE-modified at the second nucleotide from the 3′ end of the gRNA, the third nucleotide from the 3′ end of the gRNA, and the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified and 3′phosphorous-modified, e.g. 2′-O-methyl 3′thioPACE-modified at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA.

In some embodiments, the gRNA comprises a chemically modified backbone. In some embodiments, the gRNA comprises a phosphorothioate linkage. In some embodiments, one or more non-bridging oxygen atoms have been replaced with a sulfur atom. In some embodiments, the nucleotide at the 5′ end of the gRNA, the second nucleotide from the 5′ end of the gRNA, and the third nucleotide from the 5′ end of the gRNA each comprise a phosphorothioate linkage. In some embodiments, the nucleotide at the 3′ end of the gRNA, the second nucleotide from the 3′ end of the gRNA, and the third nucleotide from the 3′ end of the gRNA each comprise a phosphorothioate linkage. In some embodiments, the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA each comprise a phosphorothioate linkage. In some embodiments, the second nucleotide from the 3′ end of the gRNA, the third nucleotide from the 3′ end of the gRNA, and at the fourth nucleotide from the 3′ end of the gRNA each comprise a phosphorothioate linkage. In some embodiments, the nucleotide at the 5′ end of the gRNA, the second nucleotide from the 5′ end of the gRNA, the third nucleotide from the 5′ end, the second nucleotide from the 3′ end of the gRNA, the third nucleotide from the 3′ end of the gRNA, and the fourth nucleotide from the 3′ end of the gRNA each comprise a phosphorothioate linkage.

In some embodiments, the gRNA comprises a thioPACE linkage. In some embodiments, the gRNA comprises a backbone in which one or more non-bridging oxygen atoms have been replaced with a sulfur atom and one or more non-bridging oxygen atoms have been replaced with an acetate group. In some embodiments, the nucleotide at the 5′ end of the gRNA, the second nucleotide from the 5′ end of the gRNA, and the third nucleotide from the 5′ end of the gRNA each comprise a thioPACE linkage. In some embodiments, the nucleotide at the 3′ end of the gRNA, the second nucleotide from the 3′ end of the gRNA, and the third nucleotide from the 3′ end of the gRNA each comprise a thioPACE linkage. In some embodiments, the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA each comprise a thioPACE linkage. In some embodiments, the second nucleotide from the 3′ end of the gRNA, the third nucleotide from the 3′ end of the gRNA, and at the fourth nucleotide from the 3′ end of the gRNA each comprise a thioPACE linkage. In some embodiments, the nucleotide at the 5′ end of the gRNA, the second nucleotide from the 5′ end of the gRNA, the third nucleotide from the 5′ end, the second nucleotide from the 3′ end of the gRNA, the third nucleotide from the 3′ end of the gRNA, and the fourth nucleotide from the 3′ end of the gRNA each comprise a thioPACE linkage.

Some exemplary, non-limiting embodiments of modifications, e.g., chemical modifications, suitable for use in connection with the guide RNAs and genetic engineering methods provided herein have been described above. Additional suitable modifications, e.g., chemical modifications, will be apparent to those of skill in the art based on the present disclosure and the knowledge in the art, including, but not limited to those described in Hendel, A. et al., Nature Biotech., 2015, Vol 33, No. 9; in WO/2017/214460; in WO/2016/089433; and/or in WO/2016/164356; each one of which is herein incorporated by reference in its entirety.

gRNAs Targeting CLL1

The present disclosure provides a number of useful gRNAs that can target an endonuclease to human CLL1. Table 1 below illustrates target domains in human endogenous CLL1 that can be bound by gRNAs described herein.

TABLE 1
Exemplary target domains of human CLL1 bound
by various gRNAs are described herein. For
each target domain, the first sequence
represents a 20-nucleotide DNA sequence
corresponding to the target domain sequence
that can be targeted by a suitable gRNA, which
may comprise an equivalent RNA targeting domain
sequence (comprising RNA nucleotides instead
of the DNA nucleotides in the sequences
provided below), and the second sequence is the
reverse complement thereof. Bolding indicates
that the sequence is present in the human CLL1
cDNA sequence shown below as SEQ ID NO: 31.

| | Target Domain Sequences |
|---|---|
| gRNA A | CCCAGAAATTGGCAAATTTG (SEQ ID NO: 1) |
| | CAAATTTGCCAATTTCTGGG (SEQ ID NO: 11) |
| gRNA B | ATTCCAGAACTCCAGTGAGA (SEQ ID NO: 2) |
| | TCTCACTGGAGTTCTGGAAT (SEQ ID NO: 12) |
| gRNA C | GAGCTATATAGCAAAGAACA (SEQ ID NO: 3) |
| | TGTTCTTTGCTATATAGCTC (SEQ ID NO: 13) |
| gRNA D | GGTGGCTATTGTTTGCAGTG (SEQ ID NO: 4) |
| | CACTGCAAACAATAGCCACC (SEQ ID NO: 14) |
| gRNA E | TTGTTTGCAGTGTGGTGGAG (SEQ ID NO: 5) |
| | CTCCACCACACTGCAAACAA (SEQ ID NO: 15) |
| gRNA F | TAGCTCACGACATAATTTGG (SEQ ID NO: 6) |
| | CCAAATTATGTCGTGAGCTA (SEQ ID NO: 16) |
| gRNA G | GGCTATTGTTTGCAGTGTGG (SEQ ID NO: 7) |
| | CCACACTGCAAACAATAGCC (SEQ ID NO: 17) |
| gRNA H | GGAGAGGTTCCTGATCTTGT (SEQ ID NO: 8) |
| | ACAAGATCAGGAACCTCTCC (SEQ ID NO: 18) |
| gRNA I | TGAATATCTCCAACAAGATC (SEQ ID NO: 9) |
| | GATCTTGTTGGAGATATTCA (SEQ ID NO: 19) |
| gRNA J | GTTGTAGAGAAATATTTCTC (SEQ ID NO: 10) |
| | GAGAAATATTTCTCTACAAC (SEQ ID NO: 20) |
| gRNA O2 | ATATAATCAACTCCTCTGCC (SEQ ID NO: 40) |
| | GGCAGAGGAGTTGATTATAT (SEQ ID NO: 41) |
| gRNA P2 | AATAGACACTTACCAGGCAG (SEQ ID NO: 42) |
| | CTGCCTGGTAAGTGTCTATT (SEQ ID NO: 43) |

TABLE 2
Exemplary target domain sequences of human CLL1
bound by various gRNAs are provided herein. For
each target domain, the first sequence
represents a DNA target sequence adjacent to
a suitable PAM in the human CLL1 genomic
sequence, and the second sequence represents an
exemplary suitable gRNA targeting domain sequence.

| | Sequences | PAM |
|---|---|---|
| gRNA A | CCCAGAAATTGGCAAATTTG (SEQ ID NO: 1) | GGG |
| | CCCAGAAAUUGGCAAAUUUG (SEQ ID NO: 21) | |
| gRNA B | ATTCCAGAACTCCAGTGAGA (SEQ ID NO: 2) | TGG |
| | AUUCCAGAACUCCAGUGAGA (SEQ ID NO: 22) | |
| gRNA C | GAGCTATATAGCAAAGAACA (SEQ ID NO: 3) | AGG |
| | GAGCUAUAUAGCAAAGAACA (SEQ ID NO: 23) | |
| gRNA D | GGTGGCTATTGTTTGCAGTG (SEQ ID NO: 4) | TGG |
| | GGUGGCUAUUGUUUGCAGUG (SEQ ID NO: 24) | |
| gRNA E | TTGTTTGCAGTGTGGTGGAG (SEQ ID NO: 5) | AGG |
| | UUGUUUGCAGUGUGGUGGAG (SEQ ID NO: 25) | |
| gRNA F | TAGCTCACGACATAATTTGG (SEQ ID NO: 6) | TGG |
| | UAGCUCACGACAUAAUUUGG (SEQ ID NO: 26) | |
| gRNA G | GGCTATTGTTTGCAGTGTGG (SEQ ID NO: 7) | TGG |
| | GGCUAUUGUUUGCAGUGUGG (SEQ ID NO: 27) | |
| gRNA H | GGAGAGGTTCCTGATCTTGT (SEQ ID NO: 8) | TGG |
| | GGAGAGGUUCCUGAUCUUGU (SEQ ID NO: 28) | |
| gRNA I | TGAATATCTCCAACAAGATC (SEQ ID NO: 9) | AGG |
| | UGAAUAUCUCCAACAAGAUC (SEQ ID NO: 29) | |
| gRNA J | GTTGTAGAGAAATATTTCTC (SEQ ID NO: 10) | TGG |
| | GUUGUAGAGAAAUAUUUCUC (SEQ ID NO: 30) | |
| gRNA O2 | ATATAATCAACTCCTCTGCC (SEQ ID NO: 40) | TGG |
| | AUAUAAUCAACUCCUCUGCC (SEQ ID NO: 44) | |
| gRNA P2 | AATAGACACTTACCAGGCAG (SEQ ID NO: 42) | AGG |
| | AAUAGACACUUACCAGGCAG (SEQ ID NO: 45) | |

TABLE 6
Exemplary target domain sequences of human CLL1
bound by various gRNAs are provided herein. For
each target domain, a DNA target sequence adjacent
to a suitable PAM in the human CLL1 genomic
sequence is provided. A gRNA targeting a target
domain provided herein may comprise an equivalent
RNA sequence within its targeting domain.

| | SEQ ID NO: | Sequence | PAM |
|---|---|---|---|
| gRNA K | 67 | ATGCAGAAGTCAAATTTGAA | AGG |
| gRNA L | 68 | TCAAATTTGACTTCTGCATG | TGG |
| gRNA M | 69 | TTTGACTTCTGCATGTGGAT | AGG |
| gRNA N | 70 | GGATAGGTGAGTGTCTTTTG | TGG |
| gRNA O | 71 | TTTCCATCTCACTGGAGTTC | TGG |
| gRNA P | 72 | TGGGATTTTTTCCATCTCAC | TGG |
| gRNA Q | 73 | ATGGAAAAAATCCCAGAAAT | TGG |
| gRNA R | 74 | ATCCCAGAAATTGGCAAATT | TGG |
| gRNA S | 75 | TCCCAGAAATTGGCAAATTT | GGG |
| gRNA T | 76 | CCCCAAATTTGCCAATTTCT | GGG |
| gRNA U | 77 | TCCCCAAATTTGCCAATTTC | TGG |
| gRNA V | 78 | ATTGGCAAATTTGGGGAAAA | AGG |
| gRNA W | 79 | AAAGGTAAGATTTTGAGTTA | TGG |
| gRNA X | 80 | AGAGGGAGCTGGAGGTGCTG | TGG |
| gRNA Y | 81 | CTCCAGCTCCCTCTCATGTA | TGG |
| gRNA Z | 82 | CATACATGAGAGGGAGCTGG | AGG |
| gRNA A1 | 83 | CGCCATACATGAGAGGGAGC | TGG |
| gRNA B1 | 84 | GCTGGACGCCATACATGAGA | GGG |
| gRNA C1 | 85 | TGCTGGACGCCATACATGAG | AGG |
| gRNA D1 | 86 | AGAGTCAGAAACAAGGCTGC | TGG |
| gRNA E1 | 87 | GCACAGAAGAGTCAGAAACA | AGG |
| gRNA F1 | 88 | CTGTGCCTTCTGTTGCTCAT | TGG |
| gRNA G1 | 89 | CCTTCTGTTGCTCATTGGAT | TGG |
| gRNA H1 | 90 | CTTCTGTTGCTCATTGGATT | GGG |
| gRNA I1 | 91 | CCAATCCAATGAGCAACAGA | AGG |
| gRNA J1 | 92 | GCTCATTGGATTGGGAGTCT | TGG |
| gRNA K1 | 93 | ATATAGCTCACGACATAATT | TGG |
| gRNA L1 | 94 | AAGGTAATCTTGTATTCTCT | TGG |
| gRNA M1 | 95 | CAAGGCTTACATTTGTGCTC | TGG |
| gRNA N1 | 96 | ACAAATGTAAGCCTTGTCCA | AGG |
| gRNA O1 | 97 | GTAAGCCTTGTCCAAGGAGA | TGG |
| gRNA P1 | 98 | CTTGTCCAAGGAGATGGATT | TGG |
| gRNA Q1 | 99 | CAAATCCATCTCCTTGGACA | AGG |
| gRNA R1 | 100 | AAGGAGATGGATTTGGCATA | AGG |
| gRNA S1 | 101 | TTATGCCAAATCCATCTCCT | TGG |
| gRNA T1 | 102 | TAAGTGATGATGTCCAAACA | TGG |
| gRNA U1 | 103 | ATGTTTGGACATCATCACTT | AGG |
| gRNA V1 | 104 | TGATGATGTCCAAACATGGC | AGG |
| gRNA W1 | 105 | AACATGGCAGGAGAGTAAAA | TGG |
| gRNA X1 | 106 | TTTTACTCTCCTGCCATGTT | TGG |
| gRNA Y1 | 107 | GCTGGCATTCTGAGCAGCAC | AGG |
| gRNA Z1 | 108 | GTTGTTTATCTTCAACAGGC | TGG |
| gRNA A2 | 109 | TTTTGTTGTTTATCTTCAAC | AGG |
| gRNA B2 | 110 | GATAAACAACAAAAATGCAT | TGG |
| gRNA C2 | 111 | AAAATGCATTGGTAAAGCCC | AGG |
| gRNA D2 | 112 | AGACTATCTGTATTTCCTGT | AGG |
| gRNA E2 | 113 | GGGATTTTATAAATTCCTAC | AGG |
| gRNA F2 | 114 | AGAGTAGATCATATGACTAT | TGG |
| gRNA G2 | 115 | ATAGTCATATGATCTACTCT | GGG |
| gRNA H2 | 116 | AATAGTCATATGATCTACTC | TGG |
| gRNA I2 | 117 | TAGATCATATGACTATTGGC | TGG |
| gRNA J2 | 118 | AGATCATATGACTATTGGCT | GGG |
| gRNA K2 | 119 | CCTGAAGAAGATTCCACTCG | TGG |
| gRNA L2 | 120 | CCACGAGTGGAATCTTCTTC | AGG |
| gRNA M2 | 121 | TTCCACTCGTGGTATGAGAG | TGG |
| gRNA N2 | 122 | ATCCACTCTCATACCACGAG | TGG |
| gRNA Q2 | 123 | AACAAGAATAGACACTTACC | AGG |
| gRNA R2 | 124 | AATTCTTTACTTTCTCTTTC | AGG |
| gRNA S2 | 125 | ATTCTTTACTTTCTCTTTCA | GGG |
| gRNA T2 | 126 | GACTTAAATAACATGTATTG | TGG |
| gRNA U2 | 127 | CAATACATGTTATTTAAGTC | AGG |
| gRNA V2 | 128 | AAGAATGATATGTGAGAAGA | TGG |
| gRNA W2 | 129 | ATGGCCAATCCAGTGCAGCT | TGG |
| gRNA X2 | 130 | AGAACCAAGCTGCACTGGAT | TGG |
| gRNA Y2 | 131 | TATGTAGAACCAAGCTGCAC | TGG |
| gRNA Z2 | 132 | AGCTTGGTTCTACATATTTT | AGG |
| gRNA A3 | 133 | GCTTGGTTCTACATATTTTA | GGG |
| gRNA B3 | 134 | TGGTTCTACATATTTTAGGG | AGG |
| gRNA C3 | 135 | ACATATTTTAGGGAGGCATG | AGG |
| gRNA D3 | 136 | TATTTTGAAGATCAGAGATG | TGG |
| gRNA E3 | 137 | ATCTTCAAAATATCATGATT | TGG |
| gRNA F3 | 138 | TCTTCAAAATATCATGATTT | GGG |
| gRNA G3 | 139 | TATCATGATTTGGGTTTTCC | TGG |
| gRNA H3 | 140 | TCTCTTCTTGTTTTACTTCC | AGG |
| gRNA I3 | 141 | CTGTCCACCTCTTTGCATTA | AGG |
| gRNA J3 | 142 | CTTAATGCAAAGAGGTGGAC | AGG |
| gRNA K3 | 143 | CCTCTTTGCATTAAGGTAGT | TGG |
| gRNA L3 | 144 | ACTACCTTAATGCAAAGAGG | TGG |
| gRNA M3 | 145 | CCAACTACCTTAATGCAAAG | AGG |
| gRNA N3 | 146 | TTAAGGTAGTTGGTCCACCT | TGG |
| gRNA O3 | 147 | GAAGGCTGGCATGACCAAGG | TGG |
| gRNA P3 | 148 | GAGGAAGGCTGGCATGACCA | AGG |
| gRNA Q3 | 149 | TTAATGTGTGAGAGGAAGGC | TGG |
| gRNA R3 | 150 | TTATTTAATGTGTGAGAGGA | AGG |
| gRNA S3 | 151 | AACATTATTTAATGTGTGAG | AGG |
| gRNA T3 | 152 | ATAATGTTTGTTCTCACAGA | AGG |
| gRNA U3 | 153 | TGTTTGTTCTCACAGAAGGT | AGG |
| gRNA V3 | 154 | GTTTGTTCTCACAGAAGGTA | GGG |
| gRNA W3 | 155 | ATTGTGCTTATTTTCATAAT | GGG |
| gRNA X3 | 156 | AATTGTGCTTATTTTCATAA | TGG |
| gRNA Y3 | 157 | GAGTTTTTGGAAGATGGAAA | AGG |
| gRNA Z3 | 158 | AGGTTTGAGTTTTTGGAAGA | TGG |
| gRNA A4 | 159 | CCAAAAACTCAAACCTGTGA | AGG |
| gRNA B4 | 160 | CAAAAACTCAAACCTGTGAA | GGG |
| gRNA C4 | 161 | CCTTCACAGGTTTGAGTTTT | TGG |
| gRNA D4 | 162 | TATTCTAACTTTCCCTTCAC | AGG |
| gRNA E4 | 163 | TCAGAAAATATGTAAGTCAC | TGG |
| gRNA F4 | 164 | CTGGAAGTGGGAGGATGGCT | CGG |
| gRNA G4 | 165 | GAGGTCTGGAAGTGGGAGGA | TGG |
| gRNA H4 | 166 | GAATGAGGTCTGGAAGTGGG | AGG |
| gRNA I4 | 167 | GTCGAATGAGGTCTGGAAGT | GGG |
| gRNA J4 | 168 | AGTCGAATGAGGTCTGGAAG | TGG |
| gRNA K4 | 169 | TCCAGACCTCATTCGACTTC | TGG |
| gRNA L4 | 170 | GCCAGAAGTCGAATGAGGTC | TGG |
| gRNA M4 | 171 | ATCTCGCCAGAAGTCGAATG | AGG |
| gRNA N4 | 172 | ACTCATTTAATTCGTTGGGT | CGG |
| gRNA O4 | 173 | CCAACGAATTAAATGAGTCC | TGG |
| gRNA P4 | 174 | CAGGACTCATTTAATTCGTT | GGG |
| gRNA Q4 | 175 | CCAGGACTCATTTAATTCGT | TGG |
| gRNA R4 | 176 | AATAGGAGTACATCAAAGCC | AGG |
| gRNA S4 | 177 | TGTTTATTTATGTTTACAAT | AGG |

The CLL1 (NM_138337.6) cDNA sequence is provided below as SEQ ID NO: 31. Underlining, bolding, or italics indicates the regions complementary to gRNA A, B, C, D, E, F, G, H, I, J, or O2 (or the reverse complement thereof). Bolding and italics are used where there is overlap between two or more such regions.

(SEQ ID NO: 31)
GGCTCATTTGCAGACATATGGGTGATTGGTACAGTAGGTTTATAAACAGA
AGTTTAAACTTGTAAGCTTAAGCTTCCGTTTATAAACAGAAGTTTAAAAT
TATAGGTCCTGTTTAACATTCAGCTCTGTTAACTCACTCATCTTTTTGTG
TTTTTACACTTTGTCAAGATTTCTTTACATATTCATCAATGTCTGAAGAA
GTTACTTATGCAGATCTTCAATTCCAGAACTCCAGTGAGATGGAAAAAAT
CCCAGAAATTGGCAAATTTGGGGAAAAAGCACCTCCAGCTCCCTCTCATG
TATGGCGTCCAGCAGCCTTGTTTCTGACTCTTCTGTGCCTTCTGTTGCTC
ATTGGATTGGGAGTCTTGGCAAGCATGTTTCACGTAACTTTGAAGATAGA
AATGAAAAAAATGAACAAACTACAAAACATCAGTGAAGAGCTCCAGAGAA
ATATTTCTCTACAACTGATGAGTAACATGAATATCTCCAACAAGATCAGG
AACCICTCCACCACACTGCAAACAATAGCCACCAAATTATGTCGTGAGCT
ATATAGCAAAGAACAAGAGCACAAATGTAAGCCTTGTCCAAGGAGATGGA
TTTGGCATAAGGACAGCTGTTATTTCCTAAGTGATGATGTCCAAACATGG
CAGGAGAGTAAAATGGCCTGTGCTGCTCAGAATGCCAGCCTGTTGAAGAT
AAACAACAAAAATGCATTGGAATTTATAAAATCCCAGAGTAGATCATATG
ACTATTGGCTGGGATTATCTCCTGAAGAAGATTCCACTCGTGGTATGAGA
GTGGATAATATAATCAACTCCTCTGCCTGGGTTATAAGAAACGCACCTGA
CTTAAATAACATGTATTGTGGATATATAAATAGACTATATGTTCAATATT
ATCACTGCACTTATAAAAAAAGAATGATATGTGAGAAGATGGCCAATCCA
GTGCAGCTTGGTTCTACATATTTTAGGGAGGCATGAGGCATCAATCAAAT
ACATTTAAGGAGTGTAGGGGGTGGGGGTTCTAGGCTATAGGTAAATTTAA
ATATTTTCTGGTTGACAATTAGTTGAGTTTGTCTGAAGACCTGGGATTTT
ATCATGCAGATGAAACATCCAGGTAGCAAGCTTCAGAGAGAATAGACTGT
GAATGTTAATGCCAGAGAGGTATAATGAAGCATGTCCCACCTCCCACTTT
CCATCATGGCCTGAACCCTGGAGGAAGAGGAAGTCCATTCAGATAGTTGT
GGGGGGCCTTCGAATTTTCATTTTCATTTACGTTCTTCCCCTTCTGGCCA
AGATTTGCCAGAGGCAACATCAAAAACCAGCAAATTTTAATTTTGTCCCA
CAGCGTTGCTAGGGTGGCATGGCTCCCCATCTCGGGTCCATCCTATACTT
CCATGGGACTCCCTATGGCTGAAGGCCTTATGAGTCAAAGGACTTATAGC
CAATTGATTGTTCTAGGCCAGGTAAGAATGGATATGGACATGCATTTATT
ACCTCTTAAAATTATTATTTTAAGTAAAAGCCAATAAACAAAAACGAAAA
GGCAA

An additional CLL1 isoform (ENST00000355690.8) cDNA is provided as:

(SEQ ID NO: 46)
GGAAGAACAGCCTTTCAAATTTGACTTCTGCATGTGGATAGATTTCTTTA
CATATTCATCAATGTCTGAAGAAGTTACTTATGCAGATCTTCAATTCCAG
AACTCCAGTGAGATGGAAAAAATCCCAGAAATTGGCAAATTTGGGGAAAA
AGCACCTCCAGCTCCCTCTCATGTATGGCGTCCAGCAGCCTTGTTTCTGA
CTCTTCTGTGCCTTCTGTTGCTCATTGGATTGGGAGTCTTGGCAAGCATG
TTTCACGTAACTTTGAAGATAGAAATGAAAAAAATGAACAAACTACAAAA
CATCAGTGAAGAGCTCCAGAGAAATATTTCTCTACAACTGATGAGTAACA
TGAATATCTCCAACAAGATCAGGAACCTCTCCACCACACTGCAAACAATA
GCCACCAAATTATGTCGTGAGCTATATAGCAAAGAACAAGAGCACAAATG
TAAGCCTTGTCCAAGGAGATGGATTTGGCATAAGGACAGCTGTTATTTCC
TAAGTGATGATGTCCAAACATGGCAGGAGAGTAAAATGGCCTGTGCTGCT
CAGAATGCCAGCCTGTTGAAGATAAACAACAAAAATGCATTGGAATTTAT
AAAATCCCAGAGTAGATCATATGACTATTGGCTGGGATTATCTCCTGAAG
AAGATTCCACTCGTGGTATGAGAGTGGATAATATAATCAACTCCTCTGCC
TGGGTTATAAGAAACGCACCTGACTTAAATAACATGTATTGTGGATATAT
AAATAGACTATATGTTCAATATTATCACTGCACTTATAAAAAAAGAATGA
TATGTGAGAAGATGGCCAATCCAGTGCAGCTTGGTTCTACATATTTTAGG
GAGGCATGAGGCATCAATCAAATACATTTAAGGAGTGTAGGGGGTGGGGG
TTCTAGGCTATAGGTAAATTTAAATATTTTCTGGTTGACAATTAGTTGAG
TTTGTCTGAAGACCTGGGATTTTATCATGCAGATGAAACATCCAGGTAGC
AAGCTTCAGAGAGAATAGACTGTGAATGTTAATGCCAGAGAGGTATAATG
AAGCATGTCCCACCTCCCACTTTCCATCATGGCCTGAACCCTGGAGGAAG
AGGAAGTCCATTCAGATAGTTGTGGGGGGCCTTCGAATTTTCATTTTCAT
TTACGTTCTTCCCCTTCTGGCCAAGATTTGCCAGAGGCAACATCAAAAAC
CAGCAAATTTTAATTTTGTCCCACAGCGTTGCTAGGGTGGCATGGCTCCC
CATCTCGGGTCCATCCTATACTTCCATGGGACTCCCTATGGCTGAAGGCC
TTATGAGTCAAAGGACTTATAGCCAATTGATTGTTCTAGGCCAGGTAAGA
ATGGATATGGACATGCATTTATTACCTCTTAAAATTATTATTTTAAGTAA
AAGCCAATAAACAAAAACGAAAAGGCAA

An additional CLL1 isoform (NM_001207010.2) cDNA is provided as:

(SEQ ID NO: 270)
CTATTTAGCATTGCTGCTGCCAGCCCCAACCACATTTCTGATTGCCTAGG
AAGAACAGCCTTTCAAATTTGACTTCTGCATGTGGATAGATTTCTTTACA
TATTCATCAATGTCTGAAGAAGTTACTTATGCAGATCTTCAATTCCAGAA
CTCCAGTGAGATGGAAAAAATCCCAGAAATTGGCAAATTTGGGGAAAAAG
CACCTCCAGCTCCCTCTCATGTATGGCGTCCAGCAGCCTTGTTTCTGACT
CTTCTGTGCCTTCTGTTGCTCATTGGATTGGGAGTCTTGGCAAGCATGTT
TCACGTAACTTTGAAGATAGAAATGAAAAAAATGAACAAACTACAAAACA
TCAGTGAAGAGCTCCAGAGAAATATTTCTCTACAACTGATGAGTAACATG
AATATCTCCAACAAGATCAGGAACCTCTCCACCACACTGCAAACAATAGC
CACCAAATTATGTCGTGAGCTATATAGCAAAGAACAAGAGCACAAATGTA
AGCCTTGTCCAAGGAGATGGATTTGGCATAAGGACAGCTGTTATTTCCTA
AGTGATGATGTCCAAACATGGCAGGAGAGTAAAATGGCCTGTGCTGCTCA
GAATGCCAGCCTGTTGAAGATAAACAACAAAAATGCATTGGAATTTATAA
AATCCCAGAGTAGATCATATGACTATTGGCTGGGATTATCTCCTGAAGAA
GATTCCACTCGTGGTATGAGAGTGGATAATATAATCAACTCCTCTGCCTG
GGTTATAAGAAACGCACCTGACTTAAATAACATGTATTGTGGATATATAA
ATAGACTATATGTTCAATATTATCACTGCACTTATAAAAAAAGAATGATA
TGTGAGAAGATGGCCAATCCAGTGCAGCTTGGTTCTACATATTTTAGGGA
GGCATGAGGCATCAATCAAATACATTTAAGGAGTGTAGGGGGTGGGGGTT
CTAGGCTATAGGTAAATTTAAATATTTTCTGGTTGACAATTAGTTGAGTT
TGTCTGAAGACCTGGGATTTTATCATGCAGATGAAACATCCAGGTAGCAA
GCTTCAGAGAGAATAGACTGTGAATGTTAATGCCAGAGAGGTATAATGAA
GCATGTCCCACCTCCCACTTTCCATCATGGCCTGAACCCTGGAGGAAGAG
GAAGTCCATTCAGATAGTTGTGGGGGGCCTTCGAATTTTCATTTTCATTT
ACGTTCTTCCCCTTCTGGCCAAGATTTGCCAGAGGCAACATCAAAAACCA
GCAAATTTTAATTTTGTCCCACAGCGTTGCTAGGGTGGCATGGCTCCCCA
TCTCGGGTCCATCCTATACTTCCATGGGACTCCCTATGGCTGAAGGCCTT
ATGAGTCAAAGGACTTATAGCCAATTGATTGTTCTAGGCCAGGTAAGAAT
GGATATGGACATGCATTTATTACCTCTTAAAATTATTATTTTAAGTAAAA
GCCAATAAACAAAAACGAAAAGGCAA

An additional CLL1 isoform (NM_001300730.2) cDNA is provided as:

(SEQ ID NO: 271)
GGCTCATTTGCAGACATATGGGTGATTGGTACAGTAGGTTTATAAACAGA
AGTTTAAACTTGTAAGCTTAAGCTTCCGTTTATAAACAGAAGTTTAAAAT
TATAGGTCCTGTTTAACATTCAGCTCTGTTAACTCACTCATCTTTTTGTG
TTTTTACACTTTGTCAAGATTTCTTTACATATTCATCAATGTCTGAAGAA
GTTACTTATGCAGATCTTCAATTCCAGAACTCCAGTGAGATGGAAAAAAT
CCCAGAAATTGGCAAATTTGGGGAAAAAGCACCTCCAGCTCCCTCTCATG
TATGGCGTCCAGCAGCCTTGTTTCTGACTCTTCTGTGCCTTCTGTTGCTC
ATTGGATTGGGAGTCTTGGCAAGCATGTTTCACGTAACTTTGAAGATAGA
AATGAAAAAAATGAACAAACTACAAAACATCAGTGAAGAGCTCCAGAGAA
ATATTTCTCTACAACTGATGAGTAACATGAATATCTCCAACAAGATCAGG
AACCTCTCCACCACACTGCAAACAATAGCCACCAAATTATGTCGTGAGCT
ATATAGCAAAGAACAAGAGCACAAATGTAAGCCTTGTCCAAGGAGATGGA
TTTGGCATAAGGACAGCTGTTATTTCCTAAGTGATGATGTCCAAACATGG
CAGGAGAGTAAAATGGCCTGTGCTGCTCAGAATGCCAGCCTGTTGAAGAT
AAACAACAAAAATGCATTGGAATTTATAAAATCCCAGAGTAGATCATATG
ACTATTGGCTGGGATTATCTCCTGAAGAAGATTCCACTCGTGGTATGAGA
GTGGATAATATAATCAACTCCTCTGCCTGAAAATATCAAACGAAGAAAGA
AACCAGAGTCTCAACCTGCTGGACACTATTGGAAGTCCATCATTTAACAC
GTTTTTAGTATATACTTTTAGCAGGAGACAGCTCTGAGTCAACTGTGTTG
AGGTGCCACCACAGCGAGTTTAGGCACTCAGATCCCTGCATACTCATCAC
ATTGGGCCATAATGGCAAATAGAATTTTTTGTTTTGTTTTGTTTGTTTGC
TTTTTCTTTCACATAGAAATAGTAAGTGTAGGAGTGTGGGTCAGAAAGAA
AAGGTGGCCCTACCTCTGATGGTTGGCAATGATAGGATACAATGGGAGAT
AAGCTATCTACAAATGGAGTGGAGAAGGATATATATTTCAAAGGCCTAAT
TTGTAGTGAAAGACTAGAGACAAAGGTAATGTGTGTGTCAGGAGAGAGTA
CAGATGGAATCTTGTTTTGCAAACGTAGAATATGTATGTGTTTGTAATTA
TTGCAAATGGAATGGTAATCTATAATGGAATGGAAAACATTGTAGATATT
TTCAGTTATCAAAAAGAAAACTGAAAAAGTATATAATAATTGTATGTATG
ATATATATATGTGTGTGTGTGTGTATATATATCTTCACTTTATAACTCTG
TGTTGTTTTGGGGTTTGTTTCTGAAAGGGGGTTGTAATAAATGACATCTG
TACTATGTCACCACAAATAAATCTCATTCTTAAACATTTAATTGATGAAC
TTA

An additional CLL1 isoform (NM_201623.4) cDNA is provided as:

(SEQ ID NO: 272)
GGCTCATTTGCAGACATATGGGTGATTGGTACAGTAGGTTTATAAACAGA
AGTTTAAACTTGTAAGCTTAAGCTTCCGTTTATAAACAGAAGTTTAAAAT
TATAGGTCCTGTTTAACATTCAGCTCTGTTAACTCACTCATCTTTTTGTG
TTTTTACACTTTGTCAAGATTTCTTTACATATTCATCAATGTCTGAAGAA
GTTACTTATGCAGATCTTCAATTCCAGAACTCCAGTGAGATGGAAAAAAT
CCCAGAAATTGGCAAATTTGGGGAAAAAGTTCACGTAACTTTGAAGATAG
AAATGAAAAAAATGAACAAACTACAAAACATCAGTGAAGAGCTCCAGAGA
AATATTTCTCTACAACTGATGAGTAACATGAATATCTCCAACAAGATCAG
GAACCTCTCCACCACACTGCAAACAATAGCCACCAAATTATGTCGTGAGC
TATATAGCAAAGAACAAGAGCACAAATGTAAGCCTTGTCCAAGGAGATGG
ATTTGGCATAAGGACAGCTGTTATTTCCTAAGTGATGATGTCCAAACATG
GCAGGAGAGTAAAATGGCCTGTGCTGCTCAGAATGCCAGCCTGTTGAAGA
TAAACAACAAAAATGCATTGGAATTTATAAAATCCCAGAGTAGATCATAT
GACTATTGGCTGGGATTATCTCCTGAAGAAGATTCCACTCGTGGTATGAG
AGTGGATAATATAATCAACTCCTCTGCCTGGGTTATAAGAAACGCACCTG
ACTTAAATAACATGTATTGTGGATATATAAATAGACTATATGTTCAATAT
TATCACTGCACTTATAAAAAAAGAATGATATGTGAGAAGATGGCCAATCC
AGTGCAGCTTGGTTCTACATATTTTAGGGAGGCATGAGGCATCAATCAAA
TACATTTAAGGAGTGTAGGGGGTGGGGGTTCTAGGCTATAGGTAAATTTA
AATATTTTCTGGTTGACAATTAGTTGAGTTTGTCTGAAGACCTGGGATTT
TATCATGCAGATGAAACATCCAGGTAGCAAGCTTCAGAGAGAATAGACTG
TGAATGTTAATGCCAGAGAGGTATAATGAAGCATGTCCCACCTCCCACTT
TCCATCATGGCCTGAACCCTGGAGGAAGAGGAAGTCCATTCAGATAGTTG
TGGGGGGCCTTCGAATTTTCATTTTCATTTACGTTCTTCCCCTTCTGGCC
AAGATTTGCCAGAGGCAACATCAAAAACCAGCAAATTTTAATTTTGTCCC
ACAGCGTTGCTAGGGTGGCATGGCTCCCCATCTCGGGTCCATCCTATACT
TCCATGGGACTCCCTATGGCTGAAGGCCTTATGAGTCAAAGGACTTATAG
CCAATTGATTGTTCTAGGCCAGGTAAGAATGGATATGGACATGCATTTAT
TACCTCTTAAAATTATTATTTTAAGTAAAAGCCAATAAACAAAAACGAAA
AGGCAA

Dual gRNA Compositions and Uses Thereof

In some embodiments, a gRNA described herein (e.g., a gRNA of Table 2, 6 or 8) can be used in combination with a second gRNA, e.g., for directing nucleases to two sites in a genome. For instance, in some embodiments it is desired to produce a hematopoietic cell that is deficient for CLL1 and a second lineage-specific cell surface antigen, e.g., so that the cell can be resistant to two agents: an anti-CLL1 agent and an agent targeting the second lineage-specific cell surface antigen. In some embodiments, it is desirable to contact a cell with two different gRNAs that target different regions of CLL1, in order to make two cuts and create a deletion between the two cut sites. Accordingly, the disclosure provides various combinations of gRNAs.

In some embodiments, two or more (e.g., 3, 4, or more) gRNAs described herein are admixed. In some embodiments, each gRNA is in a separate container. In some embodiments, a kit described herein (e.g., a kit comprising one or more gRNAs according to Table 2, 6, or 8) also comprises a Cas9 molecule, or a nucleic acid encoding the Cas9 molecule.

In some embodiments, the first and second gRNAs are gRNAs according to Table 2, 6, 8 or variants thereof.

In some embodiments, the first gRNA is a CLL1 gRNA described herein (e.g., a gRNA of Table 2, 6, 8 or a variant thereof) and the second gRNA targets a lineage-specific cell-surface antigen chosen from: BCMA, CD19, CD20, CD30, ROR1, B7H6, B7H3, CD23, CD38, C-type lectin like molecule-1, CS1, IL-5, L1-CAM, PSCA, PSMA, CD138, CD133, CD70, CD7, CD13, NKG2D, NKG2D ligand, CLEC12A, CD11, CD123, CD56, CD34, CD14, CD66b, CD41, CD61, CD62, CD235a, CD146, CD326, LMP2, CD22, CD52, CD10, CD3/TCR, CD79/BCR, and CD26.

In some embodiments, the first gRNA is a CLL1 gRNA described herein (e.g., a gRNA according to Table 2, 6, 8 or a variant thereof) and the second gRNA targets a lineage-specific cell-surface antigen associated with a specific type of cancer, such as, without limitation, CD20, CD22 (Non-Hodgkin's lymphoma, B-cell lymphoma, chronic lymphocytic leukemia (CLL)), CD52 (B-cell CLL), CD33 (Acute myelogenous leukemia (AML)), CD10 (gp100) (Common (pre-B) acute lymphocytic leukemia and malignant melanoma), CD3/T-cell receptor (TCR) (T-cell lymphoma and leukemia), CD79/B-cell receptor (BCR) (B-cell lymphoma and leukemia), CD26 (epithelial and lymphoid malignancies), human leukocyte antigen (HLA)-DR, HLA-DP, and HLA-DQ (lymphoid malignancies), RCAS1 (gynecological carcinomas, biliary adenocarcinomas and ductal adenocarcinomas of the pancreas) as well as prostate specific membrane antigen.

In some embodiments, the first gRNA is a CLL1 gRNA described herein (e.g., a gRNA according to Table 2, 6, 8 or a variant thereof) and the second gRNA targets a lineage-specific cell-surface antigen chosen from: CD7, CD13, CD19, CD22, CD20, CD25, CD32, CD38, CD44, CD45, CD47, CD56, 96, CD117, CD123, CD135, CD174, CLL-1, folate receptor β, IL1RAP, MUC1, NKG2D/NKG2DL, TIM-3, or WT1.

In some embodiments, the first gRNA is a CLL1 gRNA described herein (e.g., a gRNA according to Table 2, 6, 8 or a variant thereof) and the second gRNA targets a lineage-specific cell-surface antigen chosen from: CD1a, CD1b, CD1c, CD1d, CD1e, CD2, CD3, CD3d, CD3e, CD3g, CD4, CD5, CD6, CD7, CD8a, CD8b, CD9, CD10, CD11a, CD11b, CD11c, CD11d, CDw12, CD13, CD14, CD15, CD16, CD16b, CD17, CD18, CD19, CD20, CD21, CD22, CD23, CD24, CD25, CD26, CD27, CD28, CD29, CD30, CD31, CD32a, CD32b, CD32c, CD34, CD35, CD36, CD37, CD38, CD39, CD40, CD41, CD42a, CD42b, CD42c, CD42d, CD43, CD44, CD45, CD45RA, CD45RB, CD45RC, CD45RO, CD46, CD47, CD48, CD49a, CD49b, CD49c, CD49d, CD49e, CD49f, CD50, CD51, CD52, CD53, CD54, CD55, CD56, CD57, CD58, CD59, CD60a, CD61, CD62E, CD62L, CD62P, CD63, CD64a, CD65, CD65s, CD66a, CD66b, CD66c, CD66F, CD68, CD69, CD70, CD71, CD72, CD73, CD74, CD75, CD75S, CD77, CD79a, CD79b, CD80, CD81, CD82, CD83, CD84, CD85A, CD85C, CD85D, CD85E, CD85F, CD85G, CD85H, CD85I, CD85J, CD85K, CD86, CD87, CD88, CD89, CD90, CD91, CD92, CD93, CD94, CD95, CD96, CD97, CD98, CD99, CD99R, CD100, CD101, CD102, CD103, CD104, CD105, CD106, CD107a, CD107b, CD108, CD109, CD110, CD111, CD112, CD113, CD114, CD115, CD116, CD117, CD118, CD119, CD120a, CD120b, CD121a, CD121b, CD121a, CD121b, CD122, CD123, CD124, CD125, CD126, CD127, CD129, CD130, CD131, CD132, CD133, CD134, CD135, CD136, CD137, CD138, CD139, CD140a, CD140b, CD141, CD142, CD143, CD14, CDw145, CD146, CD147, CD148, CD150, CD152, CD152, CD153, CD154, CD155, CD156a, CD156b, CD156c, CD157, CD158b1, CD158b2, CD158d, CD158e1/e2, CD158f, CD158g, CD158h, CD158i, CD158j, CD158k, CD159a, CD159c, CD160, CD161, CD163, CD164, CD165, CD166, CD167a, CD168, CD169, CD170, CD171, CD172a, CD172b, CD172g, CD173, CD174, CD175, CD175s, CD176, CD177, CD178, CD179a, CD179b, CD180, CD181, CD182, CD183, CD184, CD185, CD186, CD191, CD192, CD193, CD194, CD195, CD196, CD197, CDw198, CDw199, CD200, CD201, CD202b, CD203c, CD204, CD205, CD206, CD207, CD208, CD209, CD210a, CDw210b, CD212, CD213a1, CD213a2, CD215, CD217, CD218a, CD218b, CD220, CD221, CD222, CD223, CD224, CD225, CD226, CD227, CD228, CD229, CD230, CD231, CD232, CD233, CD234, CD235a, CD235b, CD236, CD236R, CD238, CD239, CD240, CD241, CD242, CD243, CD244, CD245, CD246, CD247, CD248, CD249, CD252, CD253, CD254, CD256, CD257, CD258, CD261, CD262, CD263, CD264, CD265, CD266, CD267, CD268, CD269, CD270, CD272, CD272, CD273, CD274, CD275, CD276, CD277, CD278, CD279, CD280, CD281, CD282, CD283, CD284, CD286, CD288, CD289, CD290, CD292, CDw293, CD294, CD295, CD296, CD297, CD298, CD299, CD300a, CD300c, CD300e, CD301, CD302, CD303, CD304, CD305, CD306, CD307a, CD307b, CD307c, CD307d, CD307e, CD309, CD312, CD314, CD315, CD316, CD317, CD318, CD319, CD320, CD321, CD322, CD324, CD325, CD326, CD327, CD328, CD329, CD331, CD332, CD333, CD334, CD335, CD336, CD337, CD338, CD339, CD340, CD344, CD349, CD350, CD351, CD352, CD353, CD354, CD355, CD357, CD358, CD359, CD360, CD361, CD362 or CD363.

In some embodiments, the first gRNA is a CLL1 gRNA described herein (e.g., a gRNA according to Table 2, 6, 8 or a variant thereof) and the second gRNA targets a lineage-specific cell-surface antigen chosen from: CD19; CD123; CD22; CD30; CD171; CS-1 (also referred to as CD2 subset 1, CRACC, SLAMF7, CD319, and 19A24); C-type lectin-like molecule-1 (CLECL1); epidermal growth factor receptor variant III (EGFRvIII); ganglioside G2 (CD2); ganglioside GD3 (aNeu5Ac(2-8)aNeu5Ac(2-3)bDGalp(1-4)bDGlep(1-1)Cer); TNF receptor family member B cell maturation (BCMA), Tn antigen ((Tn Ag) or (GalNAc.alpha.-Ser/Thr)); prostate-specific membrane antigen (PSMA); Receptor tyrosine kinase-like orphan receptor 1 (ROR1); Fms-Like tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor I receptor (IGF-I receptor), carbonic anhydrase IX (CAIX), Proteasome (Prosome, Macropain) Subunit, Beta Type 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDG1cp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex; locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1), ETS translocation-variant gene 6, located on chromosome 12p (ETV6-AML); sperm protein 17 (SPA17); X Antigen Family, member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-1AP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAXS); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxy esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2), lymphocyte antigen 75 (LY75); Glypican-3 (GPC3); Fc receptor-like 5 (FCRL5); and immunoglobulin lambda-like polypeptide 1 (IGLL1).

In some embodiments, the first gRNA is a CLL1 gRNA described herein (e.g., a gRNA according to Table 2, 6, 8 or a variant thereof) and the second gRNA targets a lineage-specific cell-surface antigen chosen from: CD11a, CD18, CD19, CD20, CD31, CD34, CD44, CD45, CD47, CD51, CD58, CD59, CD63, CD97, CD99, CD100, CD102, CD123, CD127, CD133, CD135, CD157, CD172b, CD217, CD300a, CD305, CD317, CD321, and CLL1.

In some embodiments, the first gRNA is a CLL1 gRNA described herein (e.g., a gRNA according to Table 2, 6, 8 or a variant thereof) and the second gRNA targets a lineage-specific cell-surface antigen chosen from: CD123, CLL1, CD38, CD135 (FLT3), CD56 (NCAM1), CD117 (c-KIT), FRP (FOLR2), CD47, CD82, TNFRSF1B (CD120B), CD191, CD96, PTPRJ (CD148), CD70, LILRB2 (CD85D), CD25 (IL2Ralpha), CD44, CD96, NKG2D Ligand, CD45, CD7, CD15, CD19, CD20, CD22, CD37, and CD82.

In some embodiments, the first gRNA is a CLL1 gRNA described herein (e.g., a gRNA according to Table 2, 6, 8 or a variant thereof) and the second gRNA targets a lineage-specific cell-surface antigen chosen from: CD7, CD11a, CD15, CD18, CD19, CD20, CD22, CD25, CD31, CD34, CD37, CD38, CD44, CD45, CD47, CD51, CD56, CD58, CD59, CD63, CD70, CD82, CD85D, CD96, CD97, CD99, CD100, CD102, CD117, CD120B, CD123, CD127, CD133, CD135, CD148, CD157, CD172b, CD191, CD217, CD300a, CD305, CD317, CD321, CLL1, FRP (FOLR2), or NKG2D Ligand.

In some embodiments, the first gRNA is a CLL1 gRNA described herein (e.g., a gRNA according to Table 2, 6, 8 or a variant thereof) and the second gRNA targets CD33. In some embodiments, the first gRNA is a CLL1 gRNA described herein (e.g., a gRNA according to Table 2, 6, 8 or a variant thereof) and the second gRNA targets CD123.

In some embodiments, the first gRNA is a CLL1 gRNA described herein (e.g., a gRNA according to Table 2, 6, 8 or a variant thereof) and the second gRNA comprises a sequence from Table A. In some embodiments, the first gRNA is a CLL1 gRNA comprising a targeting domain, wherein the targeting domain comprises a sequence of any of SEQ ID NOs: 1-10, 40, or 42, and the second gRNA comprises a targeting domain corresponding to a sequence of Table A. In some embodiments, the first gRNA is a CLL1 gRNA comprising a targeting domain, wherein the targeting domain comprises a sequence of SEQ ID NO: 9, and the second gRNA comprises a targeting domain corresponding to a sequence of Table A. In some embodiments, the first gRNA is a CLL1 gRNA comprising a targeting domain, wherein the targeting domain comprises a sequence of SEQ ID NO: 10, and the second gRNA comprises a targeting domain corresponding to a sequence of Table A. In some embodiments, the first gRNA is a CLL1 gRNA comprising a targeting domain, wherein the targeting domain comprises a sequence of SEQ ID NO: 11, and the second gRNA comprises a targeting domain corresponding to a sequence of Table A. In some embodiments, the first gRNA is a CLL1 gRNA comprising a targeting domain, wherein the targeting domain comprises a sequence of SEQ ID NO: 12, and the second gRNA comprises a targeting domain corresponding to a sequence of Table A. In some embodiments, the second gRNA is a gRNA disclosed in any of WO2017/066760, WO2019/046285, WO/2018/160768, or Borot et al. PNAS Jun. 11, 2019 116 (24) 11978-11987, each of which is incorporated herein by reference in its entirety.

TABLE A
Exemplary human CD33 target sequences. Certain
target sequences are followed by a PAM sequence,
indicated by a space in the text. Suitable gRNAs
binding the target sequences provided will
typically comprise a targeting domain comprising
an RNA nucleotide sequence equivalent to the
respective target sequence (and excluding the PAM).

| | | SEQ |
|---|---|---|
| gRNA target | Target sequences | ID NO: |
| hCD33 | ACCTGTCAGGTGAAGTTCGC TGG | 178 |
| hCD33 | TGGCCGGGTTCTAGAGTGCC AGG | 179 |
| hCD33 | GGCCGGGTTCTAGAGTGCCA GGG | 180 |
| hCD33 | CACCGAGGAGTGAGTAGTCC TGG | 181 |
| hCD33 | TCCAGCGAACTTCACCTGAC AGG | 182 |
| CD33 (in intron | GCTGTGGGGAGAGGGGTTGT | 183 |
| 1) | | |
| CD33 (in intron | CTGTGGGGAGAGGGGTTGTC | 184 |
| 1) | | |
| CD33 (in intron | TGGGGAAACGAGGGTCAGCT | 185 |
| 1) | | |
| CD33 (in intron | GGGCCCCTGTGGGGAAACGA | 186 |
| 1) | | |
| CD33 (in intron | AGGGCCCCTGTGGGGAAACG | 187 |
| 1) | | |
| CD33 (in intron | GCTGACCCTCGTTTCCCCAC | 188 |
| 1) | | |
| CD33 (in intron | CTGACCCTCGTTTCCCCACA | 189 |
| 1) | | |
| CD33 (in intron | TGACCCTCGTTTCCCCACAG | 190 |
| 1) | | |
| CD33 (in intron | CCATAGCCAGGGCCCCTGTG | 191 |
| 1) | | |
| CD33 (in intron | GCATGTGACAGGTGAGGCAC | 192 |
| 2) | | |
| CD33 (in intron | TGAGGCACAGGCTTCAGAAG | 193 |
| 2) | | |
| CD33 (in intron | AGGCTTCAGAAGTGGCCGCA | 194 |
| 2) | | |
| CD33 (in intron | GGCTTCAGAAGTGGCCGCAA | 195 |
| 2) | | |
| CD33 (in intron | GTACCCATGAACTTCCCTTG | 196 |
| 2) | | |
| CD33 (in intron | GTGGCCGCAAGGGAAGTTCA | 197 |
| 2) | | |
| CD33 (in intron | TGGCCGCAAGGGAAGTTCAT | 198 |
| 2) | | |
| CD33 (in intron | GGAAGTTCATGGGTACTGCA | 199 |
| 2) | | |
| CD33 (in intron | TTCATGGGTACTGCAGGGCA | 200 |
| 2) | | |
| CD33 (in intron | CTAAACCCCTCCCAGTACCA | 201 |
| 2) | | |
| CD33 (in intron | CACTCACCTGCCCACAGCAG | 202 |
| 1) | | |
| CD33 (in intron | CCCTGCTGTGGGCAGGTGAG | 203 |
| 1) | | |
| CD33 (in intron | TGGGCAGGTGAGTGGCTGTG | 204 |
| 1) | | |
| CD33 (in intron | GGTGAGTGGCTGTGGGGAGA | 205 |
| 1) | | |
| CD33 (in intron | GTGAGTGGCTGTGGGGAGAG | 206 |
| 1) | | |
| CD33 (exon 2) | ATCCATAGCCAGGGCCCCTG | 207 |
| CD33 (exon 2) | TCCATAGCCAGGGCCCCTGT | 208 |
| CD33 (exon 2) | CCATAGCCAGGGCCCCTGTG | 191 |
| CD33 (exon 2) | TCGTTTCCCCACAGGGGCCC | 209 |
| CD33 (exon 2) | TGGCTATGGATCCAAATTTC | 210 |
| CD33 (exon 2) | TGGGGAAACGAGGGTCAGCT | 185 |
| CD33 (exon 2) | GGGCCCCTGTGGGGAAACGA | 186 |
| CD33 (exon 2) | AGAAATTTGGATCCATAGCC AGG | 211 |
| CD33 (exon 3) | ATCCCTGGCACTCTAGAACC CGG | 212 |
| CD33 (exon 3) | CCTCACTAGACTTGACCCAC AGG | 213 |

### Cells Comprising Two or More Mutations

In some embodiments, an engineered cell described herein comprises two or more mutations. In some embodiments, an engineered cell described herein comprises two mutations, the first mutation being in CLL1 and the second mutation being in a second lineage-specific cell surface antigen. Such a cell can, in some embodiments, be resistant to two agents: an anti-CLL1 agent and an agent targeting the second lineage-specific cell surface antigen. In some embodiments, such a cell can be produced using two or more gRNAs described herein, e.g., a gRNA of Table 2 and a second gRNA. In some embodiments, such a cell can be produced using two or more gRNAs described herein, e.g., a gRNA of Table 6 and a second gRNA. In some embodiments, such a cell can be produced using two or more gRNAs described herein, e.g., a gRNA of Table 8 and a second gRNA. In some embodiments, the cell can be produced using, e.g., a ZFN or TALEN. The disclosure also provides populations comprising cells described herein.

In some embodiments, the second mutation is at a gene encoding a lineage-specific cell-surface antigen, e.g., one listed in the preceding section. In some embodiments, the second mutation is at a site listed in Table A.

Typically, a mutation effected by the methods and compositions provided herein, e.g., a mutation in a target gene, such as, for example, CLL1 and/or any other target gene mentioned in this disclosure, results in a loss of function of a gene product encoded by the target gene, e.g., in the case of a mutation in the CLL1 gene, in a loss of function of a CLL1 protein. In some embodiments, the loss of function is a reduction in the level of expression of the gene product, e.g., reduction to a lower level of expression, or a complete abolishment of expression of the gene product. In some embodiments, the mutation results in the expression of a non-functional variant of the gene product. For example, in the case of the mutation generating a premature stop codon in the encoding sequence, a truncated gene product, or, in the case of the mutation generating a nonsense or missense mutation, a gene product characterized by an altered amino acid sequence, which renders the gene product non-functional. In some embodiments, the function of a gene product is binding or recognition of a binding partner. In some embodiments, the reduction in expression of the gene product, e.g., of CLL1, of the second lineage-specific cell-surface antigen, or both, is to less than or equal to 50%, less than or equal to 40%, less than or equal to 30%, less than or equal to 20%, less than or equal to 10%, less than or equal to 5%, less than or equal to 2%, or less than or equal to 1% of the level in a wild-type or non-engineered counterpart cell.

In some embodiments, at least 40%, at least 50%, at least 60%, at least 70%, at least 75%, at least 80%, at least 85%, at least 90%, or at least 95% of copies of CLL1 in the population of cells generated by the methods and/or using the compositions provided herein have a mutation. In some embodiments, at least at least 40%, at least 50%, at least 60%, at least 70%, at least 75%, at least 80%, at least 85%, at least 90%, or at least 95% of copies of the second lineage-specific cell surface antigen in the population of cells have a mutation. In some embodiments, at least 40%, at least 50%, at least 60%, at least 70%, at least 75%, at least 80%, at least 85%, at least 90%, or at least 95% of copies of CLL1 and of the second lineage-specific cell surface antigen in the population of cells have a mutation. In some embodiments, the population comprises one or more wild-type cells. In some embodiments, the population comprises one or more cells that comprise one wild-type copy of CLL1. In some embodiments, the population comprises one or more cells that comprise one wild-type copy of the second lineage-specific cell surface antigen.

### Cells

In some embodiments, a cell (e.g., an HSC or HPC) having a modification of CLL1 is made using a nuclease and/or a gRNA described herein. In some embodiments, a cell (e.g., an HSC or HPC) having a modification of CLL1 and a modification of a second lineage-specific cell surface antigen is made using a nuclease and/or a gRNA described herein. It is understood that the cell can be made by contacting the cell itself with the nuclease and/or a gRNA, or the cell can be the daughter cell of a cell that was contacted with the nuclease and/or a gRNA. In some embodiments, a cell described herein (e.g., an HSC) is capable of reconstituting the hematopoietic system of a subject. In some embodiments, a cell described herein (e.g., an HSC) is capable of one or more of (e.g., all of): engrafting in a human subject, producing myeloid lineage cell, and producing and lymphoid lineage cells.

In some embodiments, a cell described herein is a human cell having a mutation in exon 2 of CLL1. In some embodiments, a cell described herein is a human cell having a mutation in exon 4 of CLL1.

In some embodiments, a population of cells described herein comprises hematopoietic stem cells (HSCs), hematopoietic progenitor cells (HPCs), or both (HSPCs). In some embodiments, the cells are CD34+.

In some embodiments, the cell comprises only one genetic modification. In some embodiments, the cell is only genetically modified at the CLL1 locus. In some embodiments, the cell is genetically modified at a second locus. In some embodiments, the cell does not comprise a transgenic protein, e.g., does not comprise a CAR.

In some embodiments, a modified cell described herein comprises substantially no CLL1 protein. In some embodiments, a modified cell described herein comprises substantially no wild-type CLL1 protein, but comprises mutant CLL1 protein. In some embodiments, the mutant CLL1 protein is not bound by an agent that targets CLL1 for therapeutic purposes.

In some embodiments, the cells are hematopoietic cells, e.g., hematopoietic stem cells. Hematopoietic stem cells (HSCs) are typically capable of giving rise to both myeloid and lymphoid progenitor cells that further give rise to myeloid cells (e.g., monocytes, macrophages, neutrophils, basophils, dendritic cells, erythrocytes, platelets, etc) and lymphoid cells (e.g., T cells, B cells, NK cells), respectively. HSCs are characterized by the expression of the cell surface marker CD34 (e.g., CD34+), which can be used for the identification and/or isolation of HSCs, and absence of cell surface markers associated with commitment to a cell lineage.

In some embodiments, a population of cells described herein comprises a plurality of hematopoietic stem cells; in some embodiments, a population of cells described herein comprises a plurality of hematopoietic progenitor cells; and in some embodiments, a population of cells described herein comprises a plurality of hematopoietic stem cells and a plurality of hematopoietic progenitor cells.

In some embodiments, the HSCs are obtained from a subject, such as a human subject. Methods of obtaining HSCs are described, e.g., in PCT/US2016/057339, which is herein incorporated by reference in its entirety. In some embodiments, the HSCs are peripheral blood HSCs. In some embodiments, the mammalian subject is a non-human primate, a rodent (e.g., mouse or rat), a bovine, a porcine, an equine, or a domestic animal. In some embodiments, the HSCs are obtained from a human subject, such as a human subject having a hematopoietic malignancy. In some embodiments, the HSCs are obtained from a healthy donor. In some embodiments, the HSCs are obtained from the subject to whom the immune cells expressing the chimeric receptors will be subsequently administered. HSCs that are administered to the same subject from which the cells were obtained are referred to as autologous cells, whereas HSCs that are obtained from a subject who is not the subject to whom the cells will be administered are referred to as allogeneic cells.

In some embodiments, at least 40%, at least 50%, at least 60%, at least 70%, at least 75%, at least 80%, at least 85%, at least 90%, or at least 95% of copies of CLL1 in the population of cells have a mutation. By way of example, a population can comprise a plurality of different CLL1 mutations and each mutation of the plurality contributes to the percent of copies of CLL1 in the population of cells that have a mutation.

In some embodiments, the expression of CLL1 on the genetically engineered hematopoietic cell is compared to the expression of CLL1 on a naturally occurring hematopoietic cell (e.g., a wild-type counterpart). In some embodiments, the genetic engineering results in a reduction in the expression level of CLL1 by at least 50%, at least 60%, at least 70%, at least 80%, at least 90%, at least 91%, at least 92%, at least 93%, at least 94%, at least 95%, at least 96%, at least 97%, at least 98%, or at least 99% as compared to the expression of CLL1 on a naturally occurring hematopoietic cell (e.g., a wild-type counterpart). For example, in some embodiments, the genetically engineered hematopoietic cell expresses less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than or 1% of CLL1 as compared to a naturally occurring hematopoietic cell (e.g., a wild-type counterpart).

In some embodiments, the genetic engineering results in a reduction in the expression level of wild-type CLL1 by at least 50%, at least 60%, at least 70%, at least 80%, at least 90%, at least 91%, at least 92%, at least 93%, at least 94%, at least 95%, at least 96%, at least 97%, at least 98%, or at least 99% as compared to the expression of the level of wild-type CLL1 on a naturally occurring hematopoietic cell (e.g., a wild-type counterpart). That is, in some embodiments, the genetically engineered hematopoietic cell expresses less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, or less than 1% of CLL1 as compared to a naturally occurring hematopoietic cell (e.g., a wild-type counterpart).

In some embodiments, the genetic engineering results in a reduction in the expression level of wild-type lineage-specific cell surface antigen (e.g., CLL1) by at least 50%, at least 60%, at least 70%, at least 80%, at least 90%, at least 91%, at least 92%, at least 93%, at least 94%, at least 95%, at least 96%, at least 97%, at least 98%, or at least 99% as compared to a suitable control (e.g., a cell or plurality of cells). In some embodiments, the suitable control comprises the level of the wild-type lineage-specific cell surface antigen measured or expected in a plurality of non-engineered cells from the same subject. In some embodiments, the suitable control comprises the level of the wild-type lineage-specific cell surface antigen measured or expected in a plurality of cells from a healthy subject. In some embodiments, the suitable control comprises the level of the wild-type lineage-specific cell surface antigen measured or expected in a population of cells from a pool of healthy individuals (e.g., 10, 20, 50, or 100 individuals). In some embodiments, the suitable control comprises the level of the wild-type lineage-specific cell surface antigen measured or expected in a subject in need of a treatment described herein, e.g., an anti-CLL1 therapy, e.g., wherein the subject has a cancer, wherein cells of the cancer express CLL1

### Methods of Treatment and Administration

In some embodiments, an effective number of CLL1-modified cells described herein is administered to a subject in combination with an anti-CLL1 therapy, e.g., an anti-CLL1 cancer therapy. In some embodiments, an effective number of cells comprising a modified CLL1 and a modified second lineage-specific cell surface antigen are administered in combination with an anti-CLL1 therapy, e.g., an anti-CLL1 cancer therapy. In some embodiments, the anti-CLL1 therapy comprises an antibody, a bispecific T cell engager, an ADC, or an immune cell expressing a CAR.

It is understood that when agents (e.g., CLL1-modified cells and an anti-CLL1 therapy) are administered in combination, the agent may be administered at the same time or at different times in temporal proximity. Furthermore, the agents may be admixed or in separate volumes. For example, in some embodiments, administration in combination includes administration in the same course of treatment, e.g., in the course of treating a cancer with an anti-CLL1 therapy, the subject may be administered an effective number of CLL1-modified cells concurrently or sequentially, e.g., before, during, or after the treatment, with the anti-CLL1 therapy.

In some embodiments, the agent that targets a CLL1 as described herein is an immune cell that expresses a chimeric receptor, which comprises an antigen-binding fragment (e.g., a single-chain antibody) capable of binding to CLL1. The immune cell may be, e.g., a T cell (e.g., a CD4+ or CD8+ T cell) or an NK cell.

A Chimeric Antigen Receptor (CAR) can comprise a recombinant polypeptide comprising at least an extracellular antigen binding domain, a transmembrane domain and a cytoplasmic signaling domain comprising a functional signaling domain, e.g., one derived from a stimulatory molecule. In one some embodiments, the cytoplasmic signaling domain further comprises one or more functional signaling domains derived from at least one costimulatory molecule, such as 4-1BB (i.e., CD137), CD27 and/or CD28 or fragments of those molecules. The extracellular antigen binding domain of the CAR may comprise a CLL1-binding antibody fragment. The antibody fragment can comprise one or more CDRs, the variable region (or portions thereof), the constant region (or portions thereof), or combinations of any of the foregoing.

Amino acid and nucleic acid sequences of an exemplary heavy chain variable region and light chain variable region of an anti-human CLL1 antibody are provided below. The CDR sequences are shown in boldface in the amino acid sequences.

Amino acid sequence of anti-CLL1 Heavy Chain
Variable Region
(SEQ ID NO: 32)
DIQLQESGPGLVKPSQSLSLTCSVTGYSITSAYYWNWIRQFPGNKLEWMG
YISYDGRNNYNPSLKNRISITRDTSKNQFFLKLNSVTTEDTATYYCAKEG
DYDVGNYYAMDYWGQGTSVTVSS
Amino acid sequence of anti-CLL1 Light Chain
Variable Region
(SEQ ID NO: 33)
ENVLTQSPAIMSASPGEKVTMTCRASSNVISSYVHWYQQRSGASPKLWIY
STSNLASGVPARFSGSGSGTSYSLTISSVEAEDAATYYCQQYSGYPLTFG
AGTKLEL

Additional anti-CLL1 sequences are found, e.g., in U.S. Pat. No. 8,536,310, which is incorporated herein by reference in its entirety.

The anti-CLL1 antibody binding fragment for use in constructing the agent that targets CLL1 as described herein may comprise the same heavy chain and/or light chain CDR regions as those in SEQ ID NO:32 and SEQ ID NO:33. Such antibodies may comprise amino acid residue variations in one or more of the framework regions. In some instances, the anti-CLL1 antibody fragment may comprise a heavy chain variable region that shares at least 70% sequence identity (e.g., 75%, 80%, 85%, 90%, 95%, or higher) with SEQ ID NO:32 and/or may comprise a light chain variable region that shares at least 70% sequence identity (e.g., 75%, 80%, 85%, 90%, 95%, or higher) with SEQ ID NO:33.

Exemplary chimeric receptor component sequences are provided in Table 3 below.

TABLE 3
Exemplary components of a chimeric receptor

| Chimeric receptor component | Amino acid sequence |
|---|---|
| Antigen-binding fragment | Light chain-GSTSSGSGKPGSGEGSTKG |
| | (SEQ ID NO: 34)-Heavy chain |
| 4-1BB costimulatory domain | KRGRKKLLYIFKQPFMRPVQTTQEEDGCSC |
| | RFPEEEEGGCE (SEQ ID NO: 214) |
| CD28 costimulatory domain | IEVMYPPPYLDNEKSNGTIIHVKGKHLCPSP |
| | LFPGPSKPFWVLVVVGGVLACYSLLVTVA |
| | FIIFWVRSKRSRLLHSDYMNMTPRRPGPTR |
| | KHYQPYAPPRDFAAYRS (SEQ ID NO: 35) |
| ICOS costimulatory domain | LSIFDPPPFKVTLTGGYLHIYESQLCCQLKF |
| (boldface), ICOS transmembrane | WLPIGCAAFVVVCILGCILICWLTKKKYSSS |
| domain (italics) and a portion | VHDPNGEYMFMRAVNTAKKSRLTDVTL |
| of the extracellular domain of | (SEQ ID NO: 36) |
| ICOS (underlined) | |
| ICOS costimulatory domain | CWLTKKKYSSSVHDPNGEYMFMRAVNTA |
| | KKSRLTDVTL (SEQ ID NO: 37) |
| CD28/ICOS chimera (the ICOS | IEVMYPPPYLDNEKSNGTIIHVKGKHLCPSPL |
| portion shown in underline) | FPGPSKPFWVLVVVGGVLACYSLLVTVA |
| including the hinge domain | FIIFWVRSKRSRLLHSDYMFMRAVNTAKK |
| (italics) and transmembrane | SRLTDVTL (SEQ ID NO: 38) |
| domain (bold) from CD28 | |
| CD8α transmembrane domain | TTTPAPRPPTPAPTIASQPLSLRPEACRPAA |
| (italics) and a portion of the | GGAVHTRGLDFACDIYIWAPLAGTCGVLLLS |
| extracellular domain of CD8α | LVITLYC (SEQ ID NO: 215) |
| (underlined) | |
| CD3ζ cytoplasmic signaling | RVKFSRSADAPAYQQGQNQLYNELNLGRR |
| domain | EEYDVLDKRRGRDPEMGGKPQRRKNPQE |
| | GLYNELQKDKMAEAYSEIGMKGERRRGK |
| | GHDGLYQGLSTATKDTYDALHMQALPPR |
| | (SEQ ID NO: 39) |

In some embodiments, the CAR comprises a 4-1BB costimulatory domain (e.g., as shown in Table 3), a CD8α transmembrane domain and a portion of the extracellular domain of CD8α (e.g., as shown in Table 3), and a CD3t cytoplasmic signaling domain (e.g., as shown in Table 3).

A typical number of cells, e.g., immune cells or hematopoietic cells, administered to a mammal (e.g., a human) can be, for example, in the range of one million to 100 billion cells; however, amounts below or above this exemplary range are also within the scope of the present disclosure.

In some embodiments, the agent that targets CLL1 is an antibody-drug conjugate (ADC). The ADC may be a molecule comprising an antibody or antigen-binding fragment thereof conjugated to a toxin or drug molecule. Binding of the antibody or fragment thereof to the corresponding antigen allows for delivery of the toxin or drug molecule to a cell that presents the antigen on the its cell surface (e.g., target cell), thereby resulting in death of the target cell.

In some embodiments, the antigen-binding fragment of the antibody-drug conjugate has the same heavy chain CDRs as the heavy chain variable region provided by SEQ ID NO: 32 and the same light chain CDRs as the light chain variable region provided by SEQ ID NO: 33. In some embodiments, the antigen-bind fragment of the antibody-drug conjugate has the heavy chain variable region provided by SEQ ID NO: 32 and the same light chain variable region provided by SEQ ID NO: 33.

Toxins or drugs compatible for use in antibody-drug conjugates are known in the art and will be evident to one of ordinary skill in the art. See, e.g., Peters et al. Biosci. Rep. (2015) 35(4): e00225; Beck et al. Nature Reviews Drug Discovery (2017) 16:315-337; Marin-Acevedo et al. J. Hematol. Oncol. (2018)11: 8; Elgundi et al. Advanced Drug Delivery Reviews (2017) 122: 2-19.

In some embodiments, the antibody-drug conjugate may further comprise a linker (e.g., a peptide linker, such as a cleavable linker) attaching the antibody and drug molecule.

Examples of antibody-drug conjugates include, without limitation, brentuximab vedotin, glembatumumab vedotin/CDX-011, depatuxizumab mafodotin/ABT-414, PSMA ADC, polatuzumab vedotin/RG7596/DCDS4501A, denintuzumab mafodotin/SGN-CD19A, AGS-16C3F, CDX-014, RG7841/DLYE5953A, RG7882/DMUC406A, RG7986/DCDS0780A, SGN-LIV1A, enfortumab vedotin/ASG-22ME, AG-15ME, AGS67E, telisotuzumab vedotin/ABBV-399, ABBV-221, ABBV-085, GSK-2857916, tisotumab vedotin/HuMax-TF-ADC, HuMax-Axl-ADC, pinatuzumab veodtin/RG7593/DCDT2980S, lifastuzumab vedotin/RG7599/DNIB0600A, indusatumab vedotin/MLN-0264/TAK-264, vandortuzumab vedotin/RG7450/DSTP3086S, sofituzumab vedotin/RG7458/DMUC5754A, RG7600/DMOT4039A, RG7336/DEDN6526A, ME1547, PF-06263507/ADC 5T4, trastuzumab emtansine/T-DM1, mirvetuximab soravtansine/IMGN853, coltuximab ravtansine/SAR3419, naratuximab emtansine/IMGN529, indatuximab ravtansine/BT-062, anetumab ravtansine/BAY 94-9343, SAR408701, SAR428926, AMG 224, PCA062, HKT288, LY3076226, SAR566658, lorvotuzumab mertansine/IMGN901, cantuzumab mertansine/SB-408075, cantuzumab ravtansine/IMGN242, laprituximab emtansine/IMGN289, IMGN388, bivatuzumab mertansine, AVE9633, BIIB015, MLN2704, AMG 172, AMG 595, LOP 628, vadastuximab talirine/SGN-CLL1A, SGN-CD70A, SGN-CD19B, SGN-CD123A, SGN-CD352A, rovalpituzumab tesirine/SC16LD6.5, SC-002, SC-003, ADCT-301/HuMax-TAC-PBD, ADCT-402, MEDI3726/ADC-401, IMGN779, IMGN632, gemtuzumab ozogamicin, inotuzumab ozogamicin/CMC-544, PF-06647263, CMD-193, CMB-401, trastuzumab duocarmazine/SYD985, BMS-936561/MDX-1203, sacituzumab govitecan/IMMU-132, labetuzumab govitecan/IMMU-130, DS-8201a, U3-1402, milatuzumab doxorubicin/IMMU-110/hLL1-DOX, BMS-986148, RC48-ADC/hertuzumab-vc-MMAE, PF-06647020, PF-06650808, PF-06664178/RN927C, lupartumab amadotin/BAY1129980, aprutumab ixadotin/BAY1187982, ARX788, AGS62P1, XMT-1522, AbGn-107, MEDI4276, DSTA4637S/RG7861. In one example, the antibody-drug conjugate is gemtuzumab ozogamicin.

In some embodiments, binding of the antibody-drug conjugate to the epitope of the cell-surface lineage-specific protein induces internalization of the antibody-drug conjugate, and the drug (or toxin) may be released intracellularly. In some embodiments, binding of the antibody-drug conjugate to the epitope of a cell-surface lineage-specific protein induces internalization of the toxin or drug, which allows the toxin or drug to kill the cells expressing the lineage-specific protein (target cells). In some embodiments, binding of the antibody-drug conjugate to the epitope of a cell-surface lineage-specific protein induces internalization of the toxin or drug, which may regulate the activity of the cell expressing the lineage-specific protein (target cells). The type of toxin or drug used in the antibody-drug conjugates described herein is not limited to any specific type.

CLL1 Associated Diseases and/or Disorders

The present disclosure provides, among other things, compositions and methods for treating a disease associated with expression of CLL1 or a condition associated with cells expressing CLL1, including, e.g., a proliferative disease such as a cancer or malignancy (e.g., a hematopoietic malignancy), or a precancerous condition such as a myelodysplasia, a myelodysplastic syndrome or a preleukemia.

In some embodiments, the hematopoietic malignancy or a hematological disorder is associated with CLL1 expression. A hematopoietic malignancy has been described as a malignant abnormality involving hematopoietic cells (e.g., blood cells, including progenitor and stem cells). Examples of hematopoietic malignancies include, without limitation, Hodgkin lymphoma, non-Hodgkin lymphoma, leukemia, or multiple myeloma. Exemplary leukemias include, without limitation, acute myeloid leukemia, acute lymphoid leukemia, chronic myelogenous leukemia, acute lymphoblastic leukemia or chronic lymphoblastic leukemia, and chronic lymphoid leukemia.

In some embodiments, cells involved in the hematopoietic malignancy are resistant to conventional or standard therapeutics used to treat the malignancy. For example, the cells (e.g., cancer cells) may be resistant to a chemotherapeutic agent and/or CAR T cells used to treat the malignancy.

In some embodiments, the leukemia is acute myeloid leukemia (AML). AML is characterized as a heterogeneous, clonal, neoplastic disease that originates from transformed cells that have progressively acquired critical genetic changes that disrupt key differentiation and growth-regulatory pathways. (Dohner et al., NEJM, (2015) 373:1136). Without wishing to be bound by theory, it is believed in some embodiments, that CLL1 is expressed on myeloid leukemia cells as well as on normal myeloid and monocytic precursors and is an attractive target for AML therapy.

In some cases, a subject may initially respond to a therapy (e.g., for a hematopoietic malignancy) and subsequently experience relapse. Any of the methods or populations of genetically engineered hematopoietic cells described herein may be used to reduce or prevent relapse of a hematopoietic malignancy. Alternatively or in addition, any of the methods described herein may involve administering any of the populations of genetically engineered hematopoietic cells described herein and an immunotherapeutic agent (e.g., cytotoxic agent) that targets cells associated with the hematopoietic malignancy and further administering one or more additional immunotherapeutic agents when the hematopoietic malignancy relapses. In some embodiments, the subject has or is susceptible to relapse of a hematopoietic malignancy (e.g., AML) following administration of one or more previous therapies. In some embodiments, the methods described herein reduce the subject's risk of relapse or the severity of relapse.

In some embodiments, the hematopoietic malignancy or hematological disorder associated with CLL1 is a precancerous condition such as a myelodysplasia, a myelodysplastic syndrome or a preleukemia. Myelodysplastic syndromes (MDS) are hematological medical conditions characterized by disorderly and ineffective hematopoiesis, or blood production. Thus, the number and quality of blood-forming cells decline irreversibly. Some patients with MDS can develop severe anemia, while others are asymptomatic. The classification scheme for MDS is known in the art, with criteria designating the ratio or frequency of particular blood cell types, e.g., myeloblasts, monocytes, and red cell precursors. MDS includes refractory anemia, refractory anemia with ring sideroblasts, refractory anemia with excess blasts, refractory anemia with excess blasts in transformation, chronic myelomonocytic leukemia (CML). In some embodiments, MDS can progress to an acute myeloid leukemia (AML).

### EXAMPLES

### Example 1: Genetic Editing of CLL1 in Human Cells

Design of sgRNA Constructs

The sgRNAs indicated in Table 4 were designed by manual inspection for the SpCas9 PAM (5′-NGG-3′) with close proximity to the target region and prioritized according to predicted specificity by minimizing potential off-target sites in the human genome with an online search algorithm (e.g., the Benchling algorithm, Doench et al 2016, Hsu et al 2013). All designed synthetic sgRNAs were produced with chemically modified nucleotides at the three terminal positions at both the 5′ and 3′ ends. Modified nucleotides contained 2′-O-methyl-3′-phosphorothioate (abbreviated as “ms”) and the ms-sgRNAs were HPLC-purified. Cas9 protein was purchased from Aldervon.

TABLE 4
Sequences of target domains of human CLL1
that can be bound by suitable gRNAs. A
corresponding gRNA will typically comprise
a targeting domain that may comprise an
equivalent RNA sequence.

| gRNA | | | |
|---|---|---|---|
| Name | Sequence | PAM | Exon |
| gRNA A | CCCAGAAATTGGCAAATTTG (SEQ ID | GGG | Exon 2 |
| | NO: 1) | | |
| gRNA B | ATTCCAGAACTCCAGTGAGA (SEQ ID | TGG | Exon 2 |
| | NO: 2) | | |
| gRNA C | GAGCTATATAGCAAAGAACA (SEQ ID | AGG | Exon 4 |
| | NO: 3) | | |
| gRNA D | GGTGGCTATTGTTTGCAGTG (SEQ ID | TGG | Exon 4 |
| | NO: 4) | | |
| gRNA E | TTGTTTGCAGTGTGGTGGAG (SEQ ID | AGG | Exon 4 |
| | NO: 5) | | |
| gRNA F | TAGCTCACGACATAATTTGG (SEQ ID | TGG | Exon 4 |
| | NO: 6) | | |
| gRNA G | GGCTATTGTTTGCAGTGTGG (SEQ ID | TGG | Exon 4 |
| | NO: 7) | | |
| gRNA H | GGAGAGGTTCCTGATCTTGT (SEQ ID | TGG | Exon 4 |
| | NO: 8) | | |
| gRNA I | TGAATATCTCCAACAAGATC (SEQ ID | AGG | Exon 4 |
| | NO: 9) | | |
| gRNA J | GTTGTAGAGAAATATTTCTC (SEQ ID | TGG | Exon 4 |
| | NO: 10) | | |

### Human CD34+ Cell Culture and Electroporation

Cryopreserved human CD34+ cells were purchased from Hemacare and thawed according to manufacturer's instructions. Human CD34+ cells were cultured for 2 days in GMP SCGM media (CellGenix), supplemented with human cytokines (Flt3, SCF, and TPO, all purchased from Peprotech). Cas9 protein and ms-sgRNA (at a 1:1 weight ratio) were mixed and incubated at room temperature for 10 minutes prior to electroporation. CD34+ cells were electroporated with the Cas9 ribonucleoprotein complex (RNP) using Lonza 4D-Nucleofector and P3 Primary Cell Kit (Program CA-137). Cells were cultured at 37° C. until analysis. Cell viability was measured by Cellometer and ViaStain AOPI Staining (Nexcelom Biosciences).

### Genomic DNA Analysis

Genomic DNA was extracted from cells 2 days post electroporation using the prepGEM DNA extraction kit (ZyGEM). The genomic region of interest was amplified by PCR.

PCR amplicons were analyzed by Sanger sequencing (Genewiz) and allele modification frequency was calculated using TIDE (Tracking of Indels by Decomposition).

### In Vitro Colony Forming Unit (CFU) Assay

Two days after electroporation, 500 CD34+ cells were plated in 1.1 mL of methylcellulose (MethoCult H4034 Optimum, Stem Cell Technologies) on 6 well plates in duplicates and cultured for two weeks. Colonies were then counted and scored using StemVision (Stem Cell Technologies).

### Results

Human CD34+ cells were electroporated with Cas9 protein and the indicated CLL1-targeting gRNA as described above.

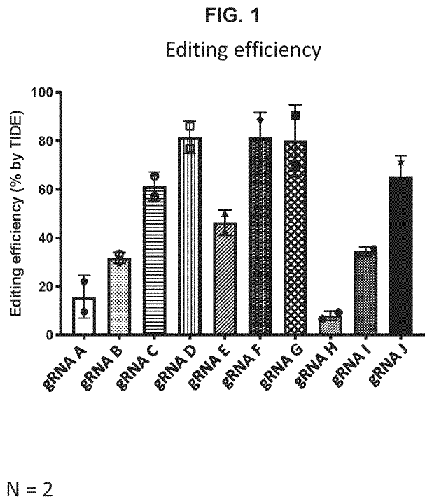

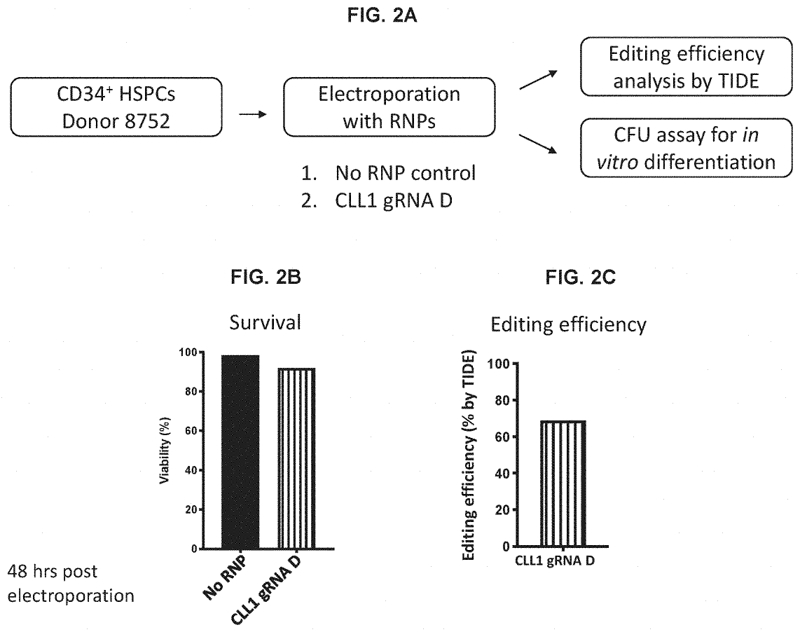

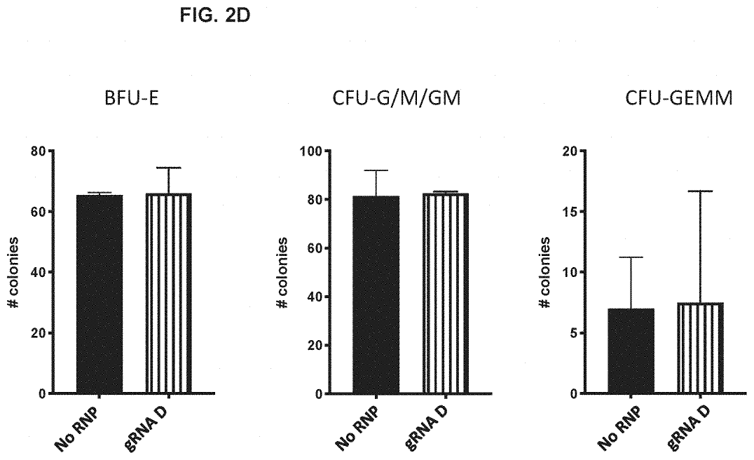

The percentage editing was determined by % INDEL as assessed by TIDE (FIGS. 1 and 2C).

As shown in FIG. 1, gRNAs D, F, and G showed a high proportion of indels, in the range of approximately 80-100% of cells. In comparison, gRNAs A and H gave much lower proportions of indels, in the range of approximately 10-20% of cells. gRNAs B, C, E, and I showed an intermediate proportion of indels, in the range of approximately 30-60% of cells.

CLL1 gRNA D was further assessed for cell viability and in vitro differentiation (FIG. 2A). As shown in FIG. 2B, cells electroporated with gRNA D showed comparable viability to negative control cells 48 hours after electroporation. These cells also showed strong editing efficiency of the CLL1/CLEC12A locus, with an indel percentage of approximately 70% (FIG. 2C). Furthermore, as shown in FIG. 2D, cells electroporated with gRNA D were able to differentiate in vitro. In particular, substantial numbers of BFU-E and CFU-G/M/GM colonies formed from cells receiving gRNA D. Lower levels of CFU-GEMM colony formation was observed in gRNA D-electroporated cells as well.

### Example 2: Generation and Evaluation of Cells Edited for Two Cell Surface Antigens

### Results

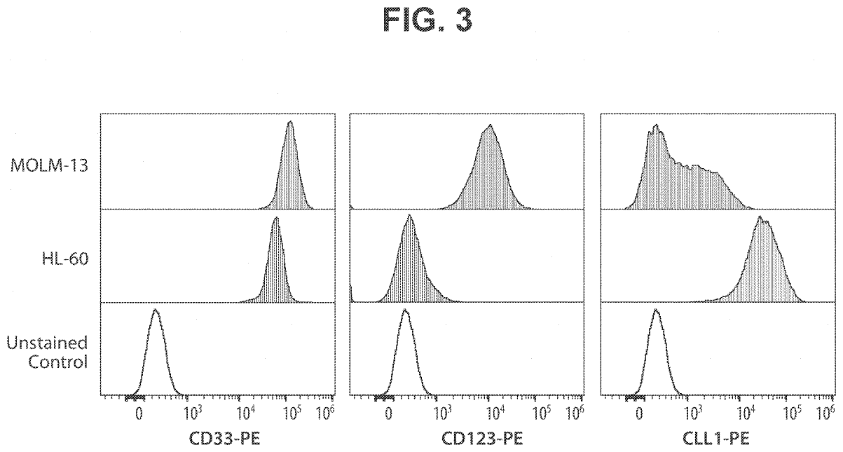

Cell surface levels of CD33, CD123 and CLL1 (CLEC12A) were measured in unedited MOLM-13 cells and THP-1 cells (both human AML cell lines) by flow cytometry. MOLM-13 cells had high levels of CD33 and CD123, and moderate-to-low levels of CLL1. HL-60 cells had high levels of CD33 and CLL1, and low levels of CD123 (FIG. 3.

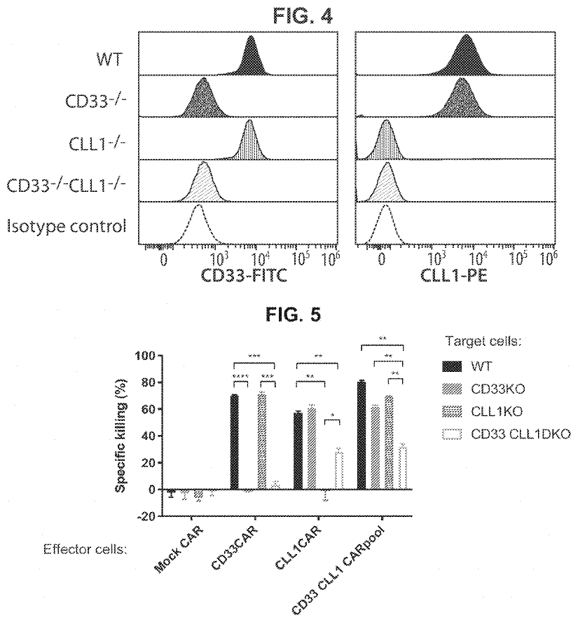

CD33 and CLL1 were mutated in HL-60 using gRNAs and Cas9 as described herein, CD33 and CLL1-modified cells were purified by flow cytometric sorting, and the cell surface levels of CD33 and CLL1 were measured. CD33 and CLL1 levels were high in wild-type HL-60 cells; editing of CD33 only resulted in low CD33 levels; editing of CLL1 only resulted in low CLL1 levels, and editing of both CD33 and CLL1 resulted in low levels of both CD33 and CLL1 (FIG. 4. The edited cells were then tested for resistance to CART effector cells using an in vitro cytotoxicity assay as described herein. All four cell types (wild-type, CD33^{-/-}, CLL1^{-/-}, and CD33^{-/-} CLL1^{-/-}) experienced low levels of specific killing in mock CAR control conditions (FIG. 5 leftmost set of bars). CD33 CAR cells effectively killed wild-type and CLL1^{-/-} cells, while CD33^{-/-} and CD33^{-/-} CLL1^{-/-} cells showed a statistically significant resistance to CD33 CAR (FIG. 5, second set of bars). CLL1 CAR cells effectively killed wild-type and CD33^{-/-} cells, while CLL1^{-/-} and CD33^{-/-} CLL1^{-/-} cells showed a statistically significant resistance to CLL1 CAR (FIG. 5, third set of bars). A pool of CD33 CAR and CLL1 CAR cells effectively killed wild-type cells, CD33^{-/-} cells, and CLL1^{-/-} cells, while CD33^{-/-} CLL1^{-/-} cells showed a statistically significant resistance to the pool of CAR cells (FIG. 5, rightmost set of bars). This experiment demonstrates that knockout of two antigens (CD33 and CLL1) protected the cells against CAR cells targeting both antigens. Furthermore, the population of edited cells contained a high enough proportion of cells that were edited at both alleles of both antigens, and had sufficiently low cell surface levels of cell surface antigens, that a statistically significant resistance to both types of CAR cells was achieved.

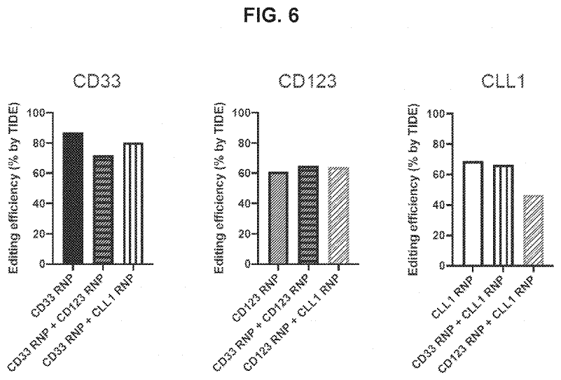

The efficiency of gene editing in human CD34+ cells was quantified using TIDE analysis as described herein. At the endogenous CD33 locus, editing efficiency of between about 70-90% was observed when CD33 was targeted alone or in combination with CD123 or CLL1 (FIG. 6, left graph). At the endogenous CD123 locus, editing efficiency of about 60% was observed when CD123 was targeted alone or in combination with CD33 or CLL1 (FIG. 6, center graph). At the endogenous CLL1 locus, editing efficiency of between about 40-70% was observed when CLL1 was targeted alone or in combination with CD33 or CD123 (FIG. 6, right graph). This experiment illustrates that human CD34+ cells can be edited at a high frequency at two cell surface antigen loci.

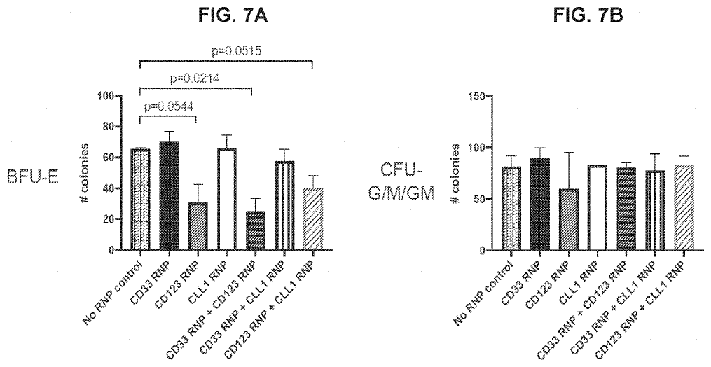

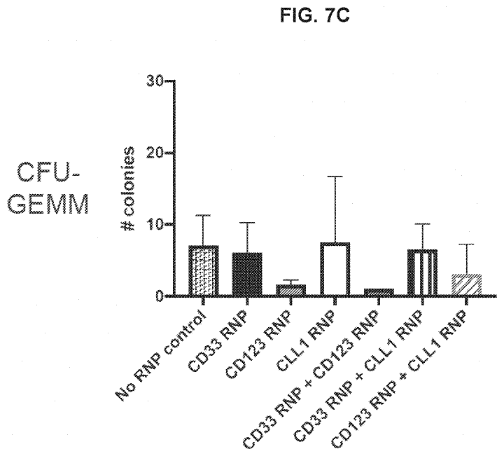

The differentiation potential of gene-edited human CD34+ cells as measured by colony formation assay as described herein. Cells edited for CD33, CD123, or CLL1, individually or in all pairwise combinations, produced BFU-E colonies, showing that the cells retain significant differentiation potential in this assay (FIG. 7A). The edited cells also produced CFU-G/M/GM colonies, showing that the cells retain differentiation potential in this assay that is statistically indistinguishable from the non-edited control (FIG. 7B). The edited cells also produced detectable CFU-GEMM colonies (FIG. 7C). Colony forming unit (CFU)-G/M/GM colonies refer to CFU-G (granulocyte), CFU-M (macrophage), and CFU-GM (granulocyte/macrophage) colonies. CFU-GEMM (granulocyte/erythroid/macrophage/megakaryocyte) colonies arise from a less differentiated cell that is a precursor to the cells that give rise to CFU-GM colonies. Taken together, the differentiation assays indicate that human CD34+ cells edited at two loci retain the capacity to differentiate into variety of cell types.

### Materials and Methods

### AML Cell Lines

Human AML cell line HL-60 was obtained from the American Type Culture Collection (ATCC). HL-60 cells were cultured in Iscove's Modified Dulbecco's Medium (IMDM, Gibco) supplemented with 20% heat-inactivated HyClone Fetal Bovine Serum (GE Healthcare). Human AML cell line MOLM-13 was obtained from AddexBio Technologies. MOLM-13 cells were cultured in RPMI-1640 media (ATCC) supplemented with 10% heat-inactivated HyClone Fetal Bovine Serum (GE Healthcare).

### Guide RNA Design

All sgRNAs were designed by manual inspection for the SpCas9 PAM (5′-NGG-3′) with close proximity to the target region and prioritized according to predicted specificity by minimizing potential off-target sites in the human genome with an online search algorithm (Benchling, Doench et al 2016, Hsu et al 2013). All designed synthetic sgRNAs were purchased from Synthego with chemically modified nucleotides at the three terminal positions at both the 5′ and 3′ ends. Modified nucleotides contained 2′-O-methyl-3′-phosphorothioate (abbreviated as “ms”) and the ms-sgRNAs were HPLC-purified. Cas9 protein was purchased from Aldevron. Typically, the gRNAs described in the Examples herein are sgRNAs comprising a 20 nucleotide (nt) targeting domain sequence, 12 nt of the crRNA repeat sequence, a 4 nt tetraloop sequence, and 64 nt of tracrRNA sequence.

TABLE 5
Sequences of target domains of human CD33,
CD123, or CLL-1 that can be bound by suitable
gRNAs. The adjacent PAM sequences are also
provided. A suitable gRNA typically comprises
a targeting domain that may comprise an RNA
sequence equivalent to the target domain
sequence.

| Target | | | |
|---|---|---|---|
| gene | Sequence | PAM | Target location |
| CD33 | CCCCAGGACTACTCACTCCT | CGG | CD33 exon 3 |
| | (SEQ ID NO: 64) | | |
| CD123 | TTTCTTGAGCTGCAGCTGGG | CGG | CD123 exon 5 |
| | (SEQ ID NO: 65) | | |
| | AGTTCCCACATCCTGGTGCG | GGG | CD123 exon 6 |
| | (SEQ ID NO: 66) | | |
| CLL1 | GGTGGCTATTGTTTGCAGTG | TGG | CLL1 exon 4 |
| | (SEQ ID NO: 4) | | |

### AML Cell Line Electroporation

Cas9 protein and ms-sgRNA (at a 1:1 weight ratio) were mixed and incubated at room temperature for 10 minutes prior to electroporation. MOLM-13 and HL-60 cells were electroporated with the Cas9 ribonucleoprotein complex (RNP) using the MaxCyte ATx Electroporator System with program THP-1 and Opt-3, respectively. Cells were incubated at 37° C. for 5-7 days until flow cytometric sorting.

### Human CD34+ Cell Culture and Electroporation

Cryopreserved human CD34+ cells were purchased from Hemacare and thawed according to manufacturer's instructions. Human CD34+ cells were cultured for 2 days in GMP SCGM media (CellGenix) supplemented with human cytokines (Flt3, SCF, and TPO, all purchased from Peprotech). CD34+ cells were electroporated with the Cas9 RNP (Cas9 protein and ms-sgRNA at a 1:1 weight ratio) using Lonza 4D-Nucleofector and P3 Primary Cell Kit (Program CA-137). For electroporation with dual ms-sgRNAs, equal amount of each ms-sgRNA was added. Cells were cultured at 37° C. until analysis.

### Genomic DNA Analysis

Genomic DNA was extracted from cells 2 days post electroporation using prepGEM DNA extraction kit (ZyGEM). Genomic region of interest was amplified by PCR. PCR amplicons were analyzed by Sanger sequencing (Genewiz) and allele modification frequency was calculated using TIDE (Tracking of Indels by Decomposition) software available on the World Wide Web at tide.deskgen.com.

### In Vitro Colony Forming Unit (CFU) Assay

Two days after electroporation, 500 CD34+ cells were plated in 1.1 mL of methylcellulose (MethoCult H4034 Optimum, Stem Cell Technologies) on 6 well plates in duplicates and cultured for two weeks. Colonies were then counted and scored using StemVision (Stem Cell Technologies).

### Flow Cytometric Analysis and Sorting

Flurochrome-conjugated antibodies against human CD33 (P67.6), CD123 (9F5), and CLL1 (REA431) were purchased from Biolegend, BD Biosciences and Miltenyi Biotec, respectively. All antibodies were tested with their respective isotype controls. Cell surface staining was performed by incubating cells with specific antibodies for 30 min on ice in the presence of human TruStain FcX. For all stains, dead cells were excluded from analysis by DAPI (Biolegend) stain. All samples were acquired and analyzed with Attune NxT flow cytometer (ThermoFisher Scientific) and FlowJo software (TreeStar).

For flow cytometric sorting, cells were stained with flurochrome-conjugated antibodies followed by sorting with Moflow Astrios Cell Sorter (Beckman Coulter).

### CAR Constructs and Lentiviral Production

Second-generation CARs were constructed to target CD33 and CLL-1, with the exception of the anti-CD33 CAR-T used in CD33/CLL-1 multiplex cytotoxicity experiment. Each CAR consisted of an extracellular scFv antigen-binding domain, using CD8α signal peptide, CD8α hinge and transmembrane regions, the 4-1BB costimulatory domain, and the CD3ξ signaling domain. The anti-CD33 scFv sequence was obtained from clone P67.6 (Mylotarg) and the CLL-1 scFv sequence from clone 1075.7. The anti-CD33 uses a heavy-to-light orientation of the scFv and the anti-CLL1 CAR construct uses a light-to-heavy orientation. The heavy and light chains were connected by (GGGS)3 linker (SEQ ID NO: 63). CAR cDNA sequences for each target were sub-cloned into the multiple cloning site of the pCDH-EF1α-MCS-T2A-GFP expression vector, and lentivirus was generated following the manufacturer's protocol (System Biosciences). Lentivirus can be generated by transient transfection of 293TN cells (System Biosciences) using Lipofectamine 3000 (ThermoFisher). The CAR construct was generated by cloning the light and heavy chain of anti-CD33 scFv (clone My96), to the CD8α hinge domain, the ICOS transmembrane domain, the ICOS signaling domain, the 4-1BB signaling domain and the CD3ξ signaling domain into the lentiviral plasmid pHIV-Zsgreen.

### CAR Transduction and Expansion

Human primary T cells were isolated from Leuko Pak (Stem Cell Technologies) by magnetic bead separation using anti-CD4 and anti-CD8 microbeads according to the manufacturer's protocol (Stem Cell Technologies). Purified CD4+ and CD8+ T cells were mixed 1:1, and activated using anti-CD3/CD28 coupled Dynabeads (Thermo Fisher) at a 1:1 bead to cell ratio. T cell culture media used was CTS Optimizer T cell expansion media supplemented with immune cell serum replacement, L-Glutamine and GlutaMAX (all purchased from Thermo Fisher) and 100 IU/mL of IL-2 (Peprotech). T cell transduction was performed 24 hours post activation by spinoculation in the presence of polybrene (Sigma). CAR-T cells were cultured for 9 days prior to cryopreservation. Prior to all experiments, T cells were thawed and rested at 37° C. for 4-6 hours.

### Flow Cytometry Based CAR-T Cytotoxicity Assay

The cytotoxicity of target cells was measured by comparing survival of target cells relative to the survival of negative control cells. For CD33/CLL1 multiplex cytotoxicity assays, wildtype and CRISPR/Cas9 edited HL60 cells were used as target cells for CD33/CLL-1 multiplex cytotoxicity assays. Wildtype Raji cell lines (ATCC) were used as negative control for both experiments. Target cells and negative control cells were stained with CellTrace Violet (CTV) and CFSE (Thermo Fisher), respectively, according to the manufacturer's instructions. After staining, target cells and negative control cells were mixed at 1:1.

Anti-CD33 or CLL1 CAR-T cells were used as effector T cells. Non-transduced T cells (mock CAR-T) were used as control. For the CARpool groups, appropriate CAR-T cells were mixed at 1:1. The effector T cells were co-cultured with the target cell/negative control cell mixture at a 1:1 effector to target ratio in duplicate. A group of target cell/negative control cell mixture alone without effector T cells was included as control. Cells were incubated at 37° C. for 24 hours before flow cytometric analysis. Propidium iodide (ThermoFisher) was used as a viability dye. For the calculation of specific cell lysis, the fraction of live target cell to live negative control cell (termed target fraction) was used. Specific cell lysis was calculated as ((target fraction without effector cells−target fraction with effector cells)/(target fraction without effectors))×100%.

### Example 3: Design and Screening of gRNAs for Editing CLL1 in Human Cells

Design of sgRNA Constructs

The gRNAs investigated in this Example were designed by inspection of the SpCas9 PAM with close proximity to the target region. All the 20 bp sequences in the coding region with an SpCas9 PAM (5′-NGG-3′) at the 3′ end were extracted. Using these methods, 123 total gRNAs targeting the target domains of human CLL1 as described in Tables 2 and 6 were designed.

Screening of gRNAs in THP-1 Cells

The 123 gRNAs were filtered according to an off-target prediction algorithm (based on number of mismatches), which identified 66 gRNAs for further investigation in THP-1 cells. Human AML cell line THP-1 was obtained from the American Type Culture Collection (ATCC). THP-1 cells were cultured and electroporated with the ribonucleoprotein RNP complexes composed of Cas9 protein and gRNA (mixed at a 1:1 weight ratio). Genomic DNA was extracted from cells and the genomic region of interest was amplified by PCR for all 66 of gRNAs investigated. PCR amplicons were then analyzed by Sanger sequencing to calculate editing frequency (ICE, or interference of CRISPR edits) in two replicates, which is shown in Table 7. In the first replicate, the editing frequency was obtained for all gRNAs that were amplified and sequenced. In the second replicate, the editing frequency was obtained for 42/66 gRNAs, and the results for each gRNA were comparable across the two replicates. As depicted in Table 7, 20 of the gRNAs investigated had an ICE value or editing frequency ≥80.

TABLE 7
Editing frequency of gRNAs designed to target human
CLL1 in THP-1 cells

| | | | Replicate | | | |
|---|---|---|---|---|---|---|
| | | | 1 | | Replicate 2 | |
| gRNA | SEQ ID NO: | Sequence | ICE | R² | ICE | R² |
| gRNA B | 22 | AUUCCAGAACUCCAGUGAGA | 62 | 0.94 | 71 | 0.95 |
| gRNA Q | 216 | AUGGAAAAAAUCCCAGAAAU | 37 | 0.97 | 36 | 0.98 |
| gRNA R | 217 | AUCCCAGAAAUUGGCAAAUU | 0 | 1 | 4 | 0.99 |
| gRNA S | 218 | UCCCAGAAAUUGGCAAAUUU | 21 | 0.98 | 21 | 0.98 |
| gRNA A | 21 | CCCAGAAAUUGGCAAAUUUG | 16 | 0.98 | 17 | 0.99 |
| gRNA V | 219 | AUUGGCAAAUUUGGGGAAAA | 27 | 0.98 | 28 | 0.98 |
| gRNA U | 220 | UCCCCAAAUUUGCCAAUUUC | 0 | 1 | 0 | 1 |
| gRNA P | 221 | UGGGAUUUUUUCCAUCUCAC | 71 | 0.93 | — | — |
| gRNA O | 222 | UUUCCAUCUCACUGGAGUUC | 79 | 0.97 | 80 | 0.96 |
| gRNA Y | 223 | CUCCAGCUCCCUCUCAUGUA | 95 | 0.95 | 96 | 0.96 |
| gRNA F1 | 224 | CUGUGCCUUCUGUUGCUCAU | 28 | 0.97 | 42 | 0.96 |
| gRNA G1 | 225 | CCUUCUGUUGCUCAUUGGAU | 76 | 0.95 | 75 | 0.97 |
| gRNA H1 | 226 | CUUCUGUUGCUCAUUGGAUU | 73 | 0.96 | 71 | 0.94 |
| gRNA J1 | 227 | GCUCAUUGGAUUGGGAGUCU | 91 | 0.93 | 94 | 0.94 |
| gRNA I1 | 228 | CCAAUCCAAUGAGCAACAGA | 66 | 0.94 | 74 | 0.94 |
| gRNA E1 | 229 | GCACAGAAGAGUCAGAAACA | 89 | 0.97 | 91 | 0.97 |
| gRNA D1 | 230 | AGAGUCAGAAACAAGGCUGC | 28 | 0.98 | 29 | 0.97 |
| gRNA C1 | 231 | UGCUGGACGCCAUACAUGAG | 88 | 0.94 | 90 | 0.94 |
| gRNA B1 | 232 | GCUGGACGCCAUACAUGAGA | 81 | 0.94 | 84 | 0.93 |
| gRNA A1 | 233 | CGCCAUACAUGAGAGGGAGC | 56 | 0.95 | 63 | 0.96 |
| gRNA Z | 234 | CAUACAUGAGAGGGAGCUGG | 80 | 0.94 | 83 | 0.94 |
| gRNA X | 235 | AGAGGGAGCUGGAGGUGCUG | 96 | 0.96 | 95 | 0.96 |
| gRNA I | 29 | UGAAUAUCUCCAACAAGAUC | 78 | 0.95 | 76 | 0.95 |
| gRNA C | 23 | GAGCUAUAUAGCAAAGAACA | 81 | 0.95 | 85 | 0.96 |
| gRNA K1 | 236 | AUAUAGCUCACGACAUAAUU | 55 | 0.98 | 53 | 0.97 |
| gRNA F | 26 | UAGCUCACGACAUAAUUUGG | 94 | 0.97 | 95 | 0.98 |
| gRNA D | 24 | GGUGGCUAUUGUUUGCAGUG | 86 | 0.95 | 85 | 0.92 |
| gRNA G | 27 | GGCUAUUGUUUGCAGUGUGG | 94 | 0.97 | 95 | 0.96 |
| gRNA E | 25 | UUGUUUGCAGUGUGGUGGAG | 50 | 0.97 | 51 | 0.97 |
| gRNA H | 28 | GGAGAGGUUCCUGAUCUUGU | 83 | 0.95 | 82 | 0.95 |
| gRNA J | 30 | GUUGUAGAGAAAUAUUUCUC | 84 | 0.96 | 88 | 0.96 |
| gRNA N1 | 237 | ACAAAUGUAAGCCUUGUCCA | 42 | 0.96 | 86 | 0.96 |
| gRNA O1 | 238 | GUAAGCCUUGUCCAAGGAGA | 77 | 0.97 | 58 | 0.96 |
| gRNA P1 | 239 | CUUGUCCAAGGAGAUGGAUU | 93 | 0.96 | 94 | 0.94 |
| gRNA R1 | 240 | AAGGAGAUGGAUUUGGCAUA | 57 | 0.92 | 59 | 0.93 |
| gRNA T1 | 241 | UAAGUGAUGAUGUCCAAACA | 36 | 0.97 | 41 | 0.97 |
| gRNA V1 | 242 | UGAUGAUGUCCAAACAUGGC | 60 | 0.96 | 59 | 0.96 |
| gRNA W1 | 243 | AACAUGGCAGGAGAGUAAAA | 58 | 0.96 | 66 | 0.97 |
| gRNA B2 | 244 | GAUAAACAACAAAAAUGCAU | 0 | 1 | 0 | 1 |
| gRNA C2 | 245 | AAAAUGCAUUGGUAAAGCCC | 85 | 0.93 | 92 | 0.96 |
| gRNA A2 | 246 | UUUUGUUGUUUAUCUUCAAC | 0 | 1 | 0 | 1 |
| gRNA Z1 | 247 | GUUGUUUAUCUUCAACAGGC | 0 | 1 | 3 | 0.99 |
| gRNA Y1 | 248 | GCUGGCAUUCUGAGCAGCAC | 69 | 0.97 | 72 | 0.95 |
| gRNA X1 | 249 | UUUUACUCUCCUGCCAUGUU | 55 | 0.95 | — | — |
| gRNA U1 | 250 | AUGUUUGGACAUCAUCACUU | 72 | 0.95 | — | — |
| gRNA S1 | 251 | UUAUGCCAAAUCCAUCUCCU | 77 | 0.97 | — | — |
| gRNA Q1 | 252 | CAAAUCCAUCUCCUUGGACA | 42 | 0.96 | — | — |
| gRNA M1 | 253 | CAAGGCUUACAUUUGUGCUC | 37 | 0.94 | — | — |
| gRNA F2 | 254 | AGAGUAGAUCAUAUGACUAU | 56 | 0.96 | — | — |
| gRNA I2 | 255 | UAGAUCAUAUGACUAUUGGC | 6 | 0.99 | — | — |
| gRNA J2 | 256 | AGAUCAUAUGACUAUUGGCU | 82 | 0.96 | — | — |
| gRNA K2 | 257 | CCUGAAGAAGAUUCCACUCG | 85 | 0.95 | — | — |
| gRNA M2 | 258 | UUCCACUCGUGGUAUGAGAG | 54 | 0.94 | — | — |
| gRNA O2 | 44 | AUAUAAUCAACUCCUCUGCC | 88 | 0.97 | — | — |
| gRNA P2 | 45 | AAUAGACACUUACCAGGCAG | 96 | 0.96 | — | — |
| gRNA N2 | 259 | AUCCACUCUCAUACCACGAG | 35 | 0.96 | — | — |
| gRNA L2 | 260 | CCACGAGUGGAAUCUUCUUC | 36 | 0.96 | — | — |
| gRNA H2 | 261 | AAUAGUCAUAUGAUCUACUC | 10 | 0.99 | — | — |
| gRNA G2 | 262 | AUAGUCAUAUGAUCUACUCU | 28 | 0.98 | — | — |
| gRNA E2 | 263 | GGGAUUUUAUAAAUUCCUAC | 0 | 1 | — | — |
| gRNA T2 | 264 | GACUUAAAUAACAUGUAUUG | 50 | 0.96 | — | |
| gRNA V2 | 265 | AAGAAUGAUAUGUGAGAAGA | 0 | 1 | — | — |
| gRNA W2 | 266 | AUGGCCAAUCCAGUGCAGCU | 88 | 0.96 | — | — |
| gRNA Y2 | 267 | UAUGUAGAACCAAGCUGCAC | 60 | 0.97 | — | — |
| gRNA X2 | 268 | AGAACCAAGCUGCACUGGAU | 79 | 0.97 | — | — |
| gRNA U2 | 269 | CAAUACAUGUUAUUUAAGUC | 37 | 0.97 | — | — |

Screening of gRNAs in Primary CD34+ Human Stem and Progenitor Cells (HSPCs)

Primary human CD34+ HSPCs were cultured and electroporated with ribonucleoprotein RNP complex composed of the Cas9 protein and one of the 14 gRNAs listed in Table 8. These 14 gRNAs screened include those that were selected from screening performed in the THP-1 cells and/or those gRNAs that had a favorable off-target profile.

TABLE 8
Sequences of target domains of CLL1 gRNAs screened in human
CD34+ cells. The corresponding gRNAs comprised a
targeting domain consisting of the equivalent RNA sequence.
The target region nomenclature is based on the CLL1 isoform
ENST00000355690.8.

| | | | | Target |
|---|---|---|---|---|
| gRNA | SEQ ID NO: | Sequence | PAM | Region |
| gRNA A | 1 | CCCAGAAATTGGCAAATTTG | GGG | Exon 2 |
| gRNA B | 2 | ATTCCAGAACTCCAGTGAGA | TGG | Exon 2 |
| gRNA C | 3 | GAGCTATATAGCAAAGAACA | AGG | Exon 4 |
| gRNA D | 4 | GGTGGCTATTGTTTGCAGTG | TGG | Exon 4 |
| gRNA E | 5 | TTGTTTGCAGTGTGGTGGAG | AGG | Exon 4 |
| gRNA F | 6 | TAGCTCACGACATAATTTGG | TGG | Exon 4 |
| gRNA G | 7 | GGCTATTGTTTGCAGTGTGG | TGG | Exon 4 |
| gRNA H | 8 | GGAGAGGTTCCTGATCTTGT | TGG | Exon 4 |
| gRNA I | 9 | TGAATATCTCCAACAAGATC | AGG | Exon 4 |
| gRNA J | 10 | GTTGTAGAGAAATATTTCTC | TGG | Exon 4 |
| gRNA K2 | 119 | CCTGAAGAAGATTCCACTCG | TGG | Exon 6 |
| gRNA O2 | 40 | ATATAATCAACTCCTCTGCC | TGG | Exon 6 |
| gRNA P1 | 98 | CTTGTCCAAGGAGATGGATT | TGG | Exon 5 |
| gRNA P2 | 42 | AATAGACACTTACCAGGCAG | AGG | Exon 6 |

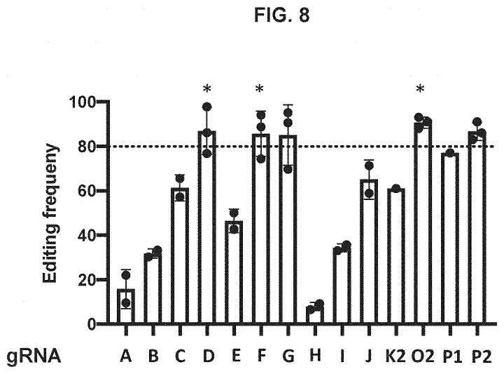

The editing frequency of these gRNAs in primary human CD34+ HSPCs was calculated and is depicted in FIG. 8 and FIG. 9. Of the 14 gRNAs tested, 5 demonstrated an editing efficiency above 80% (FIG. 8 and FIG. 9). These gRNAs included gRNA D, gRNA F, gRNA G, gRNA 02, and gRNA P2 and their calculated mean editing efficiencies are shown in Table 9.

TABLE 9
Mean editing efficiencies of gRNAs screened
in primary human CD34+ HSPCs

| Guide | Mean Editing Frequency |
|---|---|
| gRNA D | 86.9 |
| gRNA F | 85.7 |
| gRNA G | 85.1 |
| gRNA O2 | 90.7 |
| gRNA P2 | 86.7 |

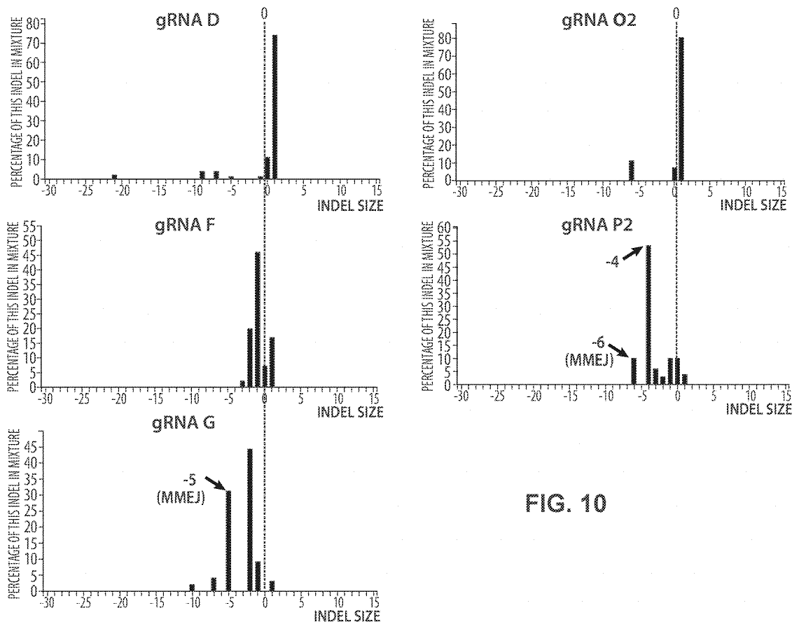

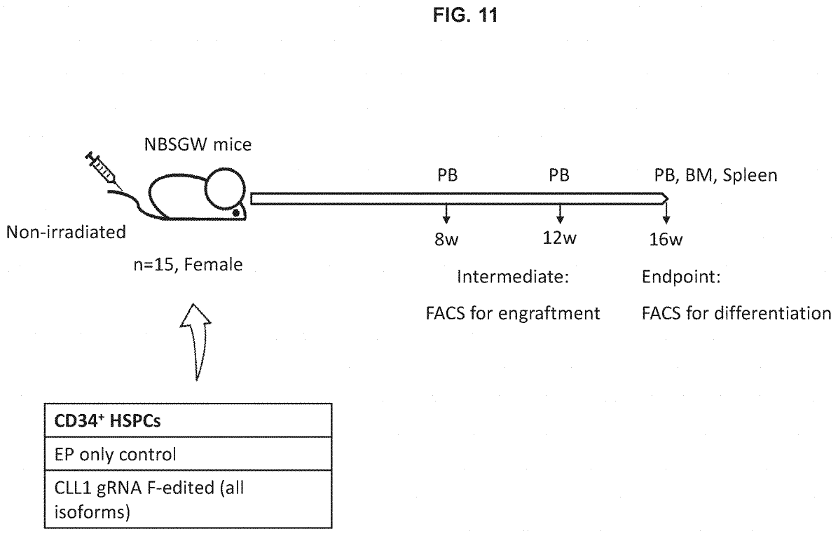

The INDEL (insertion/deletion) distributions for gRNA D, gRNA F, gRNA G, gRNA O2, and gRNA P2, as evaluated in the primary human CD34+ cells was quantified and are shown in FIG. 10. Each gRNA led to INDELs ranging from −21 to +1. gRNA D resulted INDELs of varying sizes including −21, −9, −7, −5, −1, 0, and +1, with an INDEL of +1 occurring at the highest frequency. gRNA F resulted INDELs of varying sizes including −3, −2, −1, 0, and +1, with an INDEL of −1 occurring at the highest frequency. gRNA G resulted in INDELs of varying sizes including −10, −7, −5, −2, −1, and +1, with an INDEL of −2 occurring at the highest frequency. gRNA 02 resulted in INDELs of varying sizes including −6, 0, and +1, with an INDEL of +1 occurring at the highest frequency. gRNA P2 resulted in INDELs of varying sizes including −6, −4, −3, −2, −1 0, and +1, with an INDEL of −4 occurring at the highest frequency.

The off-target effects of gRNA D, gRNA F, gRNA G, gRNA 02, and gRNA P2 were also predicted, as shown in Table 10. gRNAs were prioritized based on minimizing off-target effects. These off-target predictions were based on sequence complementarity with up to 1 nucleotide mismatch allowed between the PAM and the target or up to 3 nucleotide mismatch or gap between the guide and the target.

TABLE 10
Off-target predictions for gRNAs targeting human CLL1

| | No mismatch/gap in PAM | | 1 mismatch/gap in PAM | |
|---|---|---|---|---|
| | # of mismatch/gap in guide | | # of mismatch/gap in guide | |
| Guide | 2 | 3 | 2 | 3 |
| gRNA G | 8 | 224 | 11 | 319 |
| gRNA D | 4 | 214 | 8 | 364 |
| gRNA F | 1 | 98 | 5 | 217 |
| gRNA O2 | 2 | 168 | 20 | 441 |
| gRNA P2 | 8 | 349 | 18 | 478 |

Among other gRNAs targeting human CLL1 investigated in this Example, three gRNAs (gRNA D, gRNA F, and gRNA 02) demonstrated particularly efficient on-target editing in primary human CD34+ HSPCs, low level of predicted off-target effects, and a desirable INDEL distribution.

### Example 4: Evaluation of CLL1 KO CD34+ Cells In Vivo

### Editing in CD34+ Human HSPCs

gRNAs (Synthego) were designed as described in Example 1 and Example 3. The human CD34+ HSPCs were then edited via CRISPR/Cas9 as described in Example 1 using the CLL1 targeting guide RNA F. Non-edited, electroporated control (EP Ctrl) HPSCs were also generated.

After ex vivo editing, the genomic DNA was harvested from cells, PCR amplified with primers flanking the target region, purified, and analyzed, in order to determine their editing efficiency in the CD34+ HSPCs. As shown in Table 11, gRNA F had high editing efficiencies, specifically 75.4%.

TABLE 11
Gene editing efficiency of CLL1 gRNA

| CD34⁺ HSPCs | Editing efficiency |
|---|---|
| EP only control | N/A |
| CLL gRNA F-edited | 75.4% (TIDE) |

### Investigating Engraftment Efficiency and Persistence of CLL1 KO CD34+ HSPCs In Vivo

Female nonirradiated NOD,B6.SCID Il2rγ−/− Kit(W41/W41) (NBSGW) mice (n=15) were engrafted with the CLL1 KO HSPCs edited with gRNA F, or non-edited (EP Ctrl) (FIG. 11). At weeks 8 and 12 following engraftment, peripheral blood was collected from each mouse for analysis by FACs for measuring engraftment. At week 16, following engraftment, mice were sacrificed, and blood, spleens, and bone marrow were collected for FACS analysis for multilineage differentiation (FIG. 11).

Results from Cell Samples Obtained from the Bone Marrow of Engrafted Animals

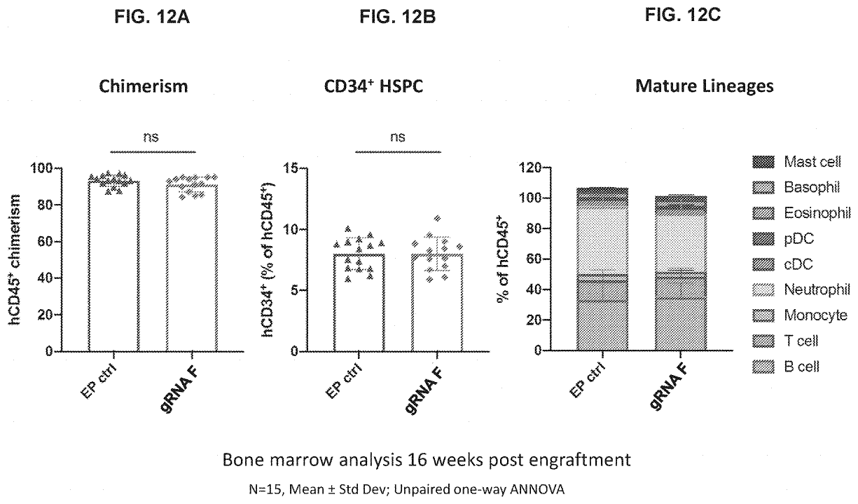

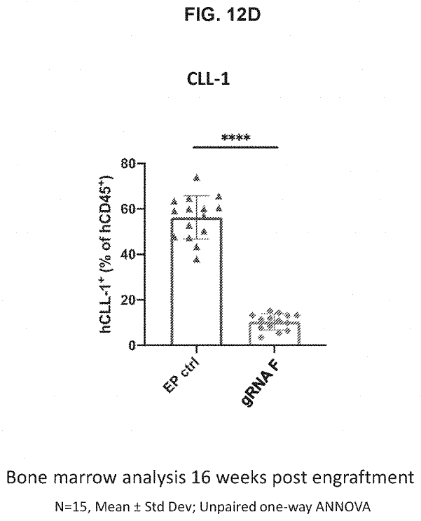

At week 16 following engraftment, rates of human leukocyte chimerism in mice were calculated as percentage of human CD45+ (hCD45+) cells in the total CD45+ cell population (the sum of human and mouse CD45+ cells) in the two groups of mice (n=15 mice/group) that received the non-edited control cells (EP Ctrl) or the CLL1 KO cells edited by gRNA F (FIG. 12A). As shown in FIG. 12A, the bone marrow chimerism was equivalent across control and CLL1 KO group, indicating no loss of nucleated bone marrow frequency.

Additionally, at week 16 post-engraftment, the percentage of hCD45+ cells that were also positive for human CD34 (hCD34+) in the bone marrow was quantified (FIG. 12B). As shown in FIG. 12B, the percentage of hCD45+ cells also expressing hCD34+ was equivalent across control or the CLL1 KO group.

At week 16 post engraftment, the percentage of hCD45+ cells that were B-cells, T cells, monocytes, neutrophils, conventional dendritic cells (cDCs), plasmacytoid dendritic cells (pDCs), eosinophils, basophils, and mast cells were quantified in the bone marrow (FIG. 12C). The percentages of these various immune cell subtypes were equivalent between the control and CLL1 KO groups. These data indicate multilineage human hematopoietic reconstitution from the edited CLL1 KO cells in the mice.

The percentages of CLL1 KO cells that were hCD45+ were quantified in the bone marrow of control and CLL1 KO cell engrafted mice at week 16 post-engraftment (FIG. 12D). The percentage of hCLL1+ hCD45+ cells was significantly lower in the CLL1 KO cells edited with gRNA F compared to the control group, indicating loss of CLL1 from nucleated blood cells in these groups. These data also demonstrate the long term persistence of CLL1 KO HSCs in the bone marrow of NBSGW mice.

### Example 5: Evaluation of CLL1 KO CD34+ Cells In Vitro

### Editing in CD34+ Human HSPCs

gRNAs (Synthego) were designed as described in Example 1 and Example 3. The human CD34+ HSPCs were then edited via CRISPR/Cas9 as described in Example 1 using the CLL1-targeting guide RNA F, as well as a non-edited, electroporated control (EP Ctrl).

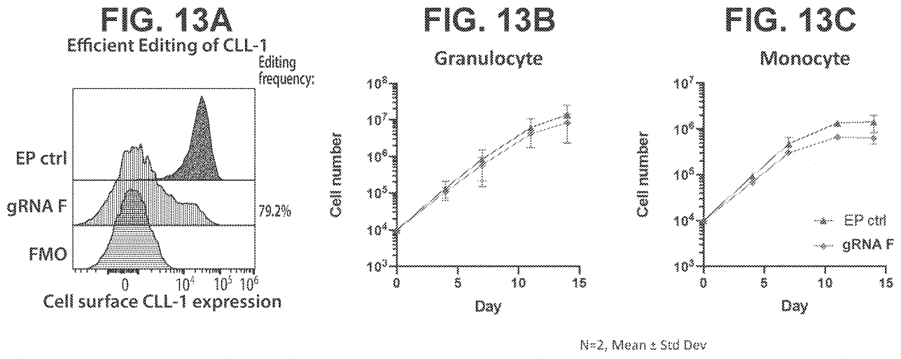

After ex vivo editing, the genomic DNA was harvested from cells, PCR amplified with primers flanking the target region, purified, and analyzed by TIDE to determine the editing frequency in the CD34+ HSPCs. As shown in FIG. 13A, gRNA F showed an editing frequency of 79.2%. Cell surface expression of CLL1 was also quantified by FACs in the CLL1 KO cells edited by gRNA F, the non-edited control (EP ctrl), or the FMO (fluorescent minus one) control. CD34+ HSPCs edited by gRNA F exhibited lower expression of CLL1 compared to the non-edited control (EP ctrl) (FIG. 13A).

Non-edited control cells (EP ctrl) or CLL1 KO cells edited by gRNA F were cultured with myeloid differentiation media, inducing either granulocytic (FIG. 13B) or monocytic (FIG. 13C) lineages, and the cell numbers were quantified over time. The CLL1 KO cells demonstrated comparable cell growth to the non-edited control cells in both granulocytic (FIG. 13B) and monocytic (FIG. 13C) differentiation culture.

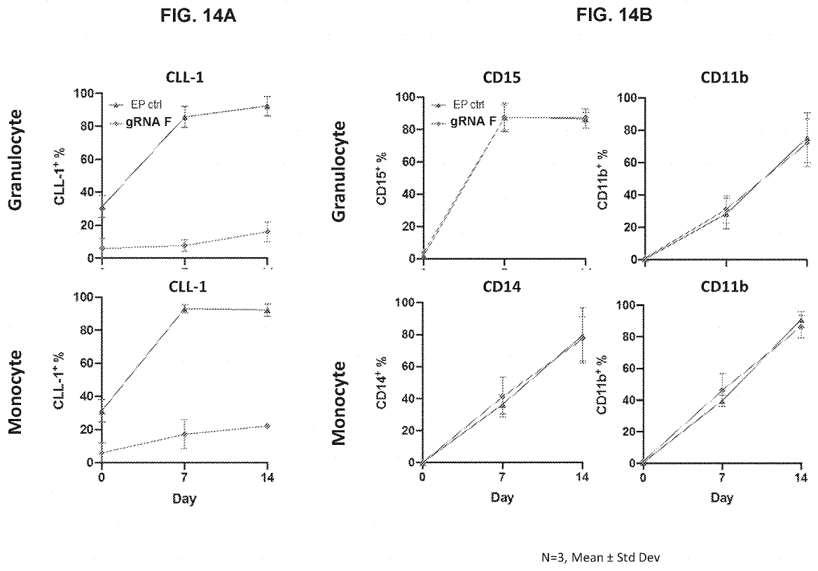

Additionally, the percentage of cells that were CLL+ in granulocytic differentiation (FIG. 14A, top) or cells that were CLL1+ in monocytic differentiation (FIG. 14A, bottom), were quantified at day 0, 7, and 14 following editing and culture of non-edited control cells or CLL1 KO cells edited by gRNA F. The granulocytes and monocytes generated from CLL1 KO cells exhibited sustained, loss of CLL1 expression over time, as compared to the non-edited control cells (FIG. 14A). The ability of the CLL1 KO cells to differentiate into myeloid cells in vitro was also evaluated. The percentage of CD15+ (FIG. 14B, top left) or CD11b+ positive granulocytes (FIG. 14B, top right) was quantified at day 0, 7, and 14 following editing and culture of non-edited control cells or CLL1KO cells edited by gRNA F. Expression of these granulocyte markers were not affected by loss of CLL1. The percentage of CD14+ (FIG. 14B, bottom left) or CD11b+ positive monocytes (FIG. 14B, bottom right) was also quantified at day 0, 7, and 14 following editing and culture of non-edited control cells or CLL1 KO cells edited by gRNA F. Similar to the granulocyte markers, expression of these monocyte markers were not affected by loss of CLL1. Expression of CD33 (marker for myeloid cells) and HLA-DR (antigen presentation) were also unaltered by CLL1 disruption. These data indicate the loss of CLL1 did not affect in vitro myeloid differentiation.

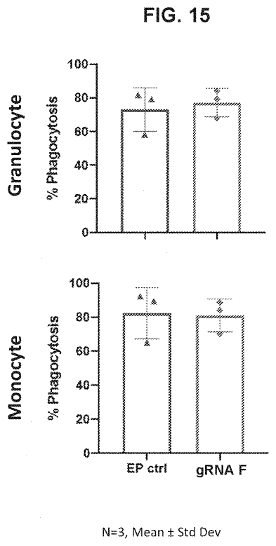

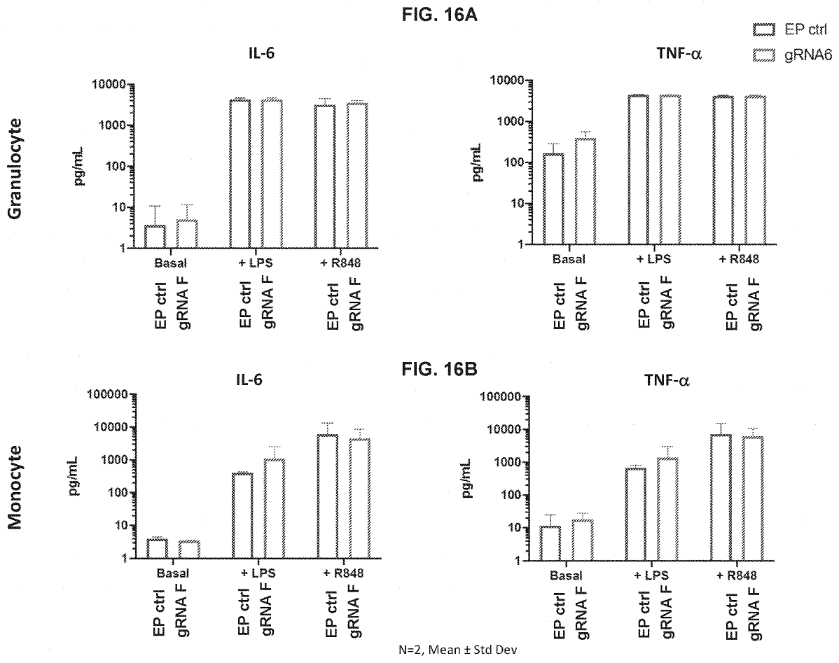

The function of CLL1 KO cells was also evaluated in vitro. The percentage of phagocytosis performed by granulocytes (FIG. 15, top) and monocytes (FIG. 15, bottom) was quantified in the control cell population and the CLL1 KO cell populations. Phagocytosis activity was equivalent between the control and CLL1 KO cells for both granulocytes and monocytes, demonstrating the CLL1 KO cells retained phagocytosis activity (FIG. 15). The ability of CLL1 KO cells to produce inflammatory cytokines upon stimulation was also evaluated. Granulocytes (FIG. 16A) and monocytes (FIG. 16B) produced from non-edited control cells or CLL1 KO cells edited by gRNA F were unstimulated or stimulated with LPS or R848. The levels of IL-6 (FIG. 16A or 16B, left) and TNF-α (FIG. 16A or 16B, right) were subsequently quantified. CLL1 KO granulocytes and monocytes exhibited intact inflammatory cytokine production upon TLR agonist stimulation and cytokine production was equivalent to non-edited control cells. Production of other cytokines, including IL-1β and MIP-1α was also not altered by CLL1 disruption. Taken together, these data demonstrate that loss of CLL1 did not affect in vitro myeloid cell function.

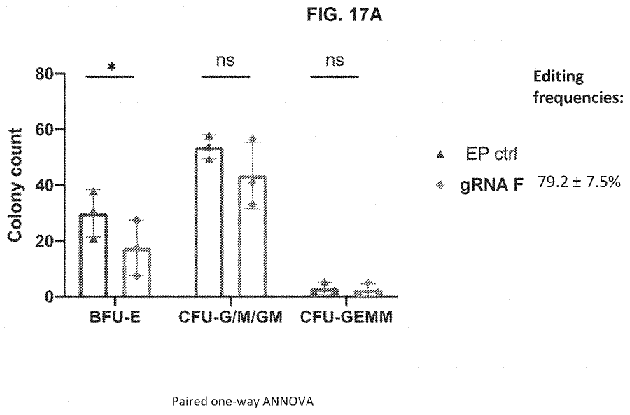

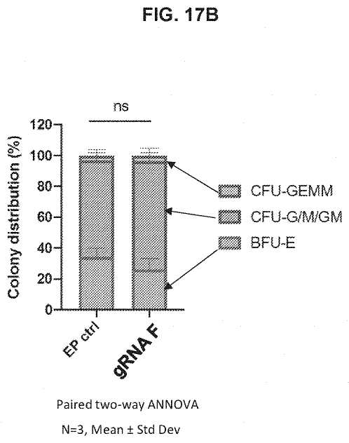

The differentiation potential of the gene-edited CD34+ CLL1 KO cells edited by gRNA F was also measured by a colony formulation assay. Following electroporation, CD34+ edited cells were plated and cultured for two weeks. Colonies were then counted and scored using StemVision (Stem Cell Technologies). Cells edited for CLL1 by gRNA F (editing frequency of 79.2%) produced fewer BFU-E, CFU-G/M/GM, and CFU-GEMM colonies compared to non-edited control cells (FIG. 17A). However, cells edited for CLL1 produced similar distributions and percentages of BFU-E colonies (Burst forming unit-erythroid), CFU-G/M/GM colonies, and CFU-GEMM colonies, as non-edited control cells, showing that the CLL1 edited cells retain significant differentiation potential in this assay (FIG. 17B). Colony forming unit (CFU)-G/M/GM colonies refer to CFU-G (granulocyte), CFU-M (macrophage), and CFU-GM (granulocyte/macrophage) colonies. CFU-GEMM (granulocyte/erythroid/macrophage/megakaryocyte) colonies arise from a less differentiated cell that is a precursor to the cells that give rise to CFU-GM colonies. Taken together, the differentiation assays indicate that human CD34+ cells edited at the CLL1 locus retain the capacity to differentiate into variety of cell types.

### Example 6 Evaluation for Resistance of CLL1 Edited Cells to CART Effector Cells

This Example describes evaluation of resistance of CLL1 edited cell to CART effector cells targeting CLL1. CLL1 KO cells that lack CLL1 expression are resistant to CLL1 CAR killing, compared to wild-type CLL1+ cells, as measured by the assays described herein.

Editing in CD34+ Human HSPCs

gRNAs (Synthego) are designed as described in Example 3. The human CD34+ HSPCs are then edited via CRISPR/Cas9 as described in Example 1 using the CLL1 targeting gRNAs, e.g., a CLL1 targeting gRNA of Table 2, 6, or 8.

CAR Constructs and Lentiviral Production

Second-generation CARs are constructed to target CLL1. The CAR consists of an extracellular scFv antigen-binding domain, using a CD8α signal peptide, a CD8α hinge and transmembrane region, a 4-1BB or CD28 costimulatory domain, and a CD3ξ signaling domain. The anti-CLL1 CAR uses the CLL-1 scFv sequence from clone 1075.7 in a light-to-heavy chain orientation. The heavy and light chains are connected by (GGGS)3 linker (SEQ ID NO: 63). The CLL1 CAR cDNA sequence is sub-cloned into the multiple cloning site of the pCDH-EF1α-MCS-T2A-GFP expression vector, and lentivirus is generated following the manufacturer's protocol (System Biosciences). Lentivirus can be generated by transient transfection of 293TN cells (System Biosciences) using Lipofectamine 3000 (ThermoFisher).

CAR Transduction and Expansion

Human primary T cells are isolated from Leuko Pak (Stem Cell Technologies) by magnetic bead separation using anti-CD4 and anti-CD8 microbeads according to the manufacturer's protocol (Stem Cell Technologies). Purified CD4+ and CD8+ T cells are mixed 1:1, and activated using anti-CD3/CD28 coupled Dynabeads (Thermo Fisher) at a 1:1 bead to cell ratio. The T cell culture media is CTS Optimizer T cell expansion media supplemented with immune cell serum replacement, L-Glutamine and GlutaMAX (all purchased from Thermo Fisher) and 100 IU/mL of IL-2 (Peprotech). T cell transduction is performed 24 hours post activation by spinoculation in the presence of polybrene (Sigma). CAR-T cells are cultured for 9 days prior to cryopreservation. Prior to all experiments, T cells are thawed and rested at 37° C. for 4-6 hours.

Flow Cytometry Based CAR-T Cytotoxicity Assay

The cytotoxicity of target cells is measured by comparing survival of target cells relative to the survival of negative control cells. For CLL1 assays, wildtype and CRISPR/Cas9 edited human CD34+ HSPCs are used as target cells. Wildtype Raji cell lines (ATCC) are used as a negative control. Target cells and negative control cells are stained with CellTrace Violet (CTV) and CFSE (Thermo Fisher), respectively, according to the manufacturer's instructions. After staining, target cells and negative control cells are mixed at 1:1.

Anti-CLL1 CAR-T cells are used as effector T cells. Non-transduced T cells (mock CAR-T) are used as control. The effector T cells are co-cultured with the target cell/negative control cell mixture at a 1:1 effector to target ratio in duplicate. A group of target cell/negative control cell mixture alone without effector T cells is included as control. Cells are incubated at 37° C. for 24 hours before flow cytometric analysis. Propidium iodide (ThermoFisher) is used as a viability dye. For the calculation of specific cell lysis, the fraction of live target cell to live negative control cell (termed target fraction) is used. Specific cell lysis is calculated as ((target fraction without effector cells−target fraction with effector cells)/(target fraction without effectors))×100%.

The analysis described above shows that CLL1 KO HPSCs (and their progeny) are resistant to anti-CLL1 CAR-T-mediated killing, while non-edited control HPSCs (and their progeny) are susceptible to anti-CLL1 CAR-T-mediated killing.

### Example 7: Treatment of Hematologic Disease

An exemplary treatment regimen using the methods, cells, and agents described herein for acute myeloid leukemia or MDS is provided. Briefly, a subject having AML or MDS that is a candidate for receiving a hematopoietic stem cell transplant (HSCT) is identified. A suitable HSC donor, e.g., an HLA-matched donor, is identified and HSCs are obtained from the donor, or, if suitable, autologous HSCs from the subject are obtained.

The HSCs so obtained are edited according to the protocols and using the strategies and compositions provided herein, e.g., a suitable guide RNA targeting a CLL1 target domain described in any of Tables 2, 6, or 8. In an exemplary embodiment, the editing is effected using a gRNA comprising a targeting domain described herein for gRNA D, gRNA F, or gRNA O2. Briefly, a targeted modification (deletion, truncation, substitution) of CLL1 is introduced via CRISPR gene editing using a suitable guide RNA and a suitable RNA-guided nuclease, e.g., a Cas9 nuclease, resulting in a loss of CLL1 expression in at least 80% of the edited HSC population.

The subject having AML or MDS may be preconditioned according to a clinical standard of care, which may include, for example, infusion of chemotherapy agents (e.g., etoposide, cyclophosphamide) and/or irradiation. Depending on the health status of the subject and the status of disease progression in the subject, such pre-conditioning may be omitted, however.

A CLL1-targeted immunotherapy, e.g., a CAR-T cell therapy targeting CLL1 is administered to the subject. The edited HSCs from the donor or the edited HSCs from the subject are administered to the subject, and engraftment, survival, and/or differentiation of the HSCs into mature cells of the hematopoietic lineages in the subject are monitored. The CLL1-targeted immunotherapy selectively targets and kills CLL1 expressing malignant or pre-malignant cells, and may also target some healthy cells expressing CLL1 in the subject, but does not target the edited HSCs or their progeny in the subject, as these cells are resistant to targeting and killing by a CLL1-targeted immunotherapy.

The health status and disease progression in the subject is monitored regularly after administration of the immunotherapy and edited HSCs to confirm a reduction in the burden of CLL1-expressing malignant or pre-malignant cells, and to confirm successful engraftment of the edited HSCs and their progeny.

### EQUIVALENTS AND SCOPE

Those skilled in the art will recognize, or be able to ascertain using no more than routine experimentation, many equivalents of the exemplary embodiments described herein. The scope of the present disclosure is not intended to be limited to the above description.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the invention encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, it is to be understood that every possible individual element or subgroup of the elements is also disclosed, and that any element or subgroup of elements can be removed from the group. It is also noted that the term “comprising” is intended to be open and permits the inclusion of additional elements, features, or steps. It should be understood that, in general, where an embodiment, is referred to as comprising particular elements, features, or steps, embodiments, that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

All publications, patent applications, patents, and other references (e.g., sequence database reference numbers) mentioned herein are incorporated by reference in their entirety. For example, all GenBank, Unigene, and Entrez sequences referred to herein, e.g., in any Table herein, are incorporated by reference. Unless otherwise specified, the sequence accession numbers specified herein, including in any Table herein, refer to the database entries current as of Aug. 28, 2019. When one gene or protein references a plurality of sequence accession numbers, all of the sequence variants are encompassed.

In addition, it is to be understood that any particular embodiment of the present invention may be explicitly excluded from any one or more of the claims. Where ranges are given, any value within the range may explicitly be excluded from any one or more of the claims. For purposes of brevity, all of the embodiments in which one or more elements, features, purposes, or aspects is excluded are not set forth explicitly herein.

1. A gRNA comprising a targeting domain, wherein the targeting domain comprises a sequence of SEQ ID NO: 21.

2. A gRNA comprising a targeting domain, wherein the targeting domain comprises a sequence of SEQ ID NO: 22.

3. A gRNA comprising a targeting domain, wherein the targeting domain comprises a sequence of SEQ ID NO: 23.

4. A gRNA comprising a targeting domain, wherein the targeting domain comprises a sequence of SEQ ID NO: 24.

5. A gRNA comprising a targeting domain, wherein the targeting domain comprises a sequence of SEQ ID NO: 25.

6. A gRNA comprising a targeting domain, wherein the targeting domain comprises a sequence of SEQ ID NO: 26.

7. A gRNA comprising a targeting domain, wherein the targeting domain comprises a sequence of SEQ ID NO: 27.

8. A gRNA comprising a targeting domain, wherein the targeting domain comprises a sequence of SEQ ID NO: 28.

9. A gRNA comprising a targeting domain, wherein the targeting domain comprises a sequence of SEQ ID NO: 29.

10. A gRNA comprising a targeting domain, wherein the targeting domain comprises a sequence of SEQ ID NO: 30.

11. A gRNA comprising a targeting domain, wherein the targeting domain comprises a sequence of SEQ ID NO: 44.

12. A gRNA comprising a targeting domain, wherein the targeting domain comprises a sequence of SEQ ID NO: 45.

13. A gRNA comprising a targeting domain, wherein the targeting domain comprises a sequence of SEQ ID NO: 257.

14. A gRNA comprising a targeting domain, wherein the targeting domain comprises a sequence of SEQ ID NO: 239.

15. A gRNA comprising a targeting domain which binds a target domain of Table 1, 2, 6, or 8.

16. A gRNA comprising a targeting domain capable of directing cleavage or editing of a target domain of Table 1, 2, 6, or 8.

17. The gRNA of any of claim 1-16, which comprises a first complementarity domain, a linking domain, a second complementarity domain which is complementary to the first complementarity domain, and a proximal domain.

18. The gRNA of any of claims 1-17, which is a single guide RNA (sgRNA).

19. The gRNA of any of claims 1-18, which comprises one or more 2′O-methyl nucleotide.

20. The gRNA of any of claims 1-19, which comprises one or more phosphorothioate or thioPACE linkage.

21. A method of producing a genetically engineered cell, comprising:
(i) providing a cell (e.g., a hematopoietic stem or progenitor cell, e.g., a wild-type hematopoietic stem or progenitor cell), and
(ii) introducing into the cell (a) a gRNA of any of claims 1-17; and (b) a Cas9 molecule that binds the gRNA,
thereby producing the genetically engineered cell.

22. The method of claim 21, wherein the Cas molecule comprises a SpCas9 endonuclease, a SaCas9 endonuclease, or a Cpf1 endonuclease.

23. The method of claim 21 or 22, wherein (i) and (ii) are introduced into the cell as a pre-formed ribonucleoprotein complex.

24. The method of claim 21, wherein the ribonucleoprotein complex is introduced into the cell via electroporation.

25. A genetically engineered hematopoietic stem or progenitor cell, which is produced by a method of claim 21.

26. A cell population, comprising a plurality of the genetically engineered hematopoietic stem or progenitor cells of claim 25.

27. The cell population of claim 26, which further comprises one or more cells that comprise one or more non-engineered CLL1 genes.

28. The cell population of claim 26 or 27, which expresses less than 20% of the CLL1 expressed by a wild-type counterpart cell population.

29. The cell population of any of claims 26-28, which comprises both of hematopoietic stem cells and hematopoietic progenitor cells.

30. The cell population of any of claims 26-29, which further comprises a second mutation at a gene encoding a lineage-specific cell surface antigen other than CLL1.

31. The cell population of claim 30, wherein the gene encoding a lineage-specific cell surface antigen other than CLL1 is CD33 or CD123.

32. A method, comprising administering to a subject in need thereof a cell population of any of claims 26-31.

33. The method of claim 32, wherein the subject has a hematopoietic malignancy.

34. The method of claim 32 or 33, which further comprises administering to the subject an effective amount of an agent that targets CLL1, wherein the agent comprises an antigen-binding fragment that binds CLL1.


SEQUENCE LISTING (272 sequences)

SEQ ID NO: 1 - DNA, 20 bp, Homo sapiens
cccagaaattggcaaatttg

SEQ ID NO: 2 - DNA, 20 bp, Homo sapiens
attccagaactccagtgaga

SEQ ID NO: 3 - DNA, 20 bp, Homo sapiens
gagctatatagcaaagaaca

SEQ ID NO: 4 - DNA, 20 bp, Homo sapiens
ggtggctattgtttgcagtg

SEQ ID NO: 5 - DNA, 20 bp, Homo sapiens
ttgtttgcagtgtggtggag

SEQ ID NO: 6 - DNA, 20 bp, Homo sapiens
tagctcacgacataatttgg

SEQ ID NO: 7 - DNA, 20 bp, Homo sapiens
ggctattgtttgcagtgtgg

SEQ ID NO: 8 - DNA, 20 bp, Homo sapiens
ggagaggttcctgatcttgt

SEQ ID NO: 9 - DNA, 20 bp, Homo sapiens
tgaatatctccaacaagatc

SEQ ID NO: 10 - DNA, 20 bp, Homo sapiens
gttgtagagaaatatttctc

SEQ ID NO: 11 - DNA, 20 bp, Homo sapiens
caaatttgccaatttctggg

SEQ ID NO: 12 - DNA, 20 bp, Homo sapiens
tctcactggagttctggaat

SEQ ID NO: 13 - DNA, 20 bp, Homo sapiens
tgttctttgctatatagctc

SEQ ID NO: 14 - DNA, 20 bp, Homo sapiens
cactgcaaacaatagccacc

SEQ ID NO: 15 - DNA, 20 bp, Homo sapiens
ctccaccacactgcaaacaa

SEQ ID NO: 16 - DNA, 20 bp, Homo sapiens
ccaaattatgtcgtgagcta

SEQ ID NO: 17 - DNA, 20 bp, Homo sapiens
ccacactgcaaacaatagcc

SEQ ID NO: 18 - DNA, 20 bp, Homo sapiens
acaagatcaggaacctctcc

SEQ ID NO: 19 - DNA, 20 bp, Homo sapiens
gatcttgttggagatattca

SEQ ID NO: 20 - DNA, 20 bp, Homo sapiens
gagaaatatttctctacaac

SEQ ID NO: 21 - RNA, 20 nt, Homo sapiens
cccagaaauuggcaaauuug

SEQ ID NO: 22 - RNA, 20 nt, Homo sapiens
auuccagaacuccagugaga

SEQ ID NO: 23 - RNA, 20 nt, Homo sapiens
gagcuauauagcaaagaaca

SEQ ID NO: 24 - RNA, 20 nt, Homo sapiens
gguggcuauuguuugcagug

SEQ ID NO: 25 - RNA, 20 nt, Homo sapiens
uuguuugcagugugguggag

SEQ ID NO: 26 - RNA, 20 nt, Homo sapiens
uagcucacgacauaauuugg

SEQ ID NO: 27 - RNA, 20 nt, Homo sapiens
ggcuauuguuugcagugugg

SEQ ID NO: 28 - RNA, 20 nt, Homo sapiens
ggagagguuccugaucuugu

SEQ ID NO: 29 - RNA, 20 nt, Homo sapiens
ugaauaucuccaacaagauc

SEQ ID NO: 30 - RNA, 20 nt, Homo sapiens
guuguagagaaauauuucuc

SEQ ID NO: 31 - DNA, 1555 bp, Homo sapiens
ggctcatttgcagacatatgggtgattggtacagtaggtttataaacagaagtttaaacttgtaagcttaagcttccgtttataaacagaagtttaaaattataggtcctgtttaacattcagctctgttaactcactcatctttttgtgtttttacactttgtcaagatttctttacatattcatcaatgtctgaagaagttacttatgcagatcttcaattccagaactccagtgagatggaaaaaatcccagaaattggcaaatttggggaaaaagcacctccagctccctctcatgtatggcgtccagcagccttgtttctgactcttctgtgccttctgttgctcattggattgggagtcttggcaagcatgtttcacgtaactttgaagatagaaatgaaaaaaatgaacaaactacaaaacatcagtgaagagctccagagaaatatttctctacaactgatgagtaacatgaatatctccaacaagatcaggaacctctccaccacactgcaaacaatagccaccaaattatgtcgtgagctatatagcaaagaacaagagcacaaatgtaagccttgtccaaggagatggatttggcataaggacagctgttatttcctaagtgatgatgtccaaacatggcaggagagtaaaatggcctgtgctgctcagaatgccagcctgttgaagataaacaacaaaaatgcattggaatttataaaatcccagagtagatcatatgactattggctgggattatctcctgaagaagattccactcgtggtatgagagtggataatataatcaactcctctgcctgggttataagaaacgcacctgacttaaataacatgtattgtggatatataaatagactatatgttcaatattatcactgcacttataaaaaaagaatgatatgtgagaagatggccaatccagtgcagcttggttctacatattttagggaggcatgaggcatcaatcaaatacatttaaggagtgtagggggtgggggttctaggctataggtaaatttaaatattttctggttgacaattagttgagtttgtctgaagacctgggattttatcatgcagatgaaacatccaggtagcaagcttcagagagaatagactgtgaatgttaatgccagagaggtataatgaagcatgtcccacctcccactttccatcatggcctgaaccctggaggaagaggaagtccattcagatagttgtggggggccttcgaattttcattttcatttacgttcttccccttctggccaagatttgccagaggcaacatcaaaaaccagcaaattttaattttgtcccacagcgttgctagggtggcatggctccccatctcgggtccatcctatacttccatgggactccctatggctgaaggccttatgagtcaaaggacttatagccaattgattgttctaggccaggtaagaatggatatggacatgcatttattacctcttaaaattattattttaagtaaaagccaataaacaaaaacgaaaaggcaa

SEQ ID NO: 32 - protein, 123 aa, Artificial Sequence
  source: /note=“Description of Artificial Sequence: Synthetic polypeptide”
DIQLQESGPGLVKPSQSLSLTCSVTGYSITSAYYWNWIRQFPGNKLEWMGYISYDGRNNYNPSLKNRISITRDTSKNQFFLKLNSVTTEDTATYYCAKEGDYDVGNYYAMDYWGQGTSVTVSS

SEQ ID NO: 33 - protein, 107 aa, Artificial Sequence
  source: /note=“Description of Artificial Sequence: Synthetic polypeptide”
ENVLTQSPAIMSASPGEKVTMTCRASSNVISSYVHWYQQRSGASPKLWIYSTSNLASGVPARFSGSGSGTSYSLTISSVEAEDAATYYCQQYSGYPLTFGAGTKLEL

SEQ ID NO: 34 - protein, 19 aa, Artificial Sequence
  source: /note=“Description of Artificial Sequence: Synthetic peptide”
GSTSSGSGKPGSGEGSTKG

SEQ ID NO: 35 - protein, 107 aa, Artificial Sequence
  source: /note=“Description of Artificial Sequence: Synthetic polypeptide”
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 36 - protein, 89 aa, Artificial Sequence
  source: /note=“Description of Artificial Sequence: Synthetic polypeptide”
LSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 37 - protein, 38 aa, Artificial Sequence
  source: /note=“Description of Artificial Sequence: Synthetic polypeptide”
CWLTKKKYSSSVHDPNGEYMFMRAVNTAKKSRLTDVTL

SEQ ID NO: 38 - protein, 97 aa, Artificial Sequence
  source: /note=“Description of Artificial Sequence: Synthetic polypeptide”
IEVMYPPPYLDNEKSNGTIIHVKGKHLCPSPLFPGPSKPFWVLVVVGGVLACYSLLVTVAFIIFWVRSKRSRLLHSDYMFMRAVNTAKKSRLTDVTL

SEQ ID NO: 39 - protein, 113 aa, Artificial Sequence
  source: /note=“Description of Artificial Sequence: Synthetic polypeptide”
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 40 - DNA, 20 bp, Homo sapiens
atataatcaactcctctgcc

SEQ ID NO: 41 - DNA, 20 bp, Homo sapiens
ggcagaggagttgattatat

SEQ ID NO: 42 - DNA, 20 bp, Homo sapiens
aatagacacttaccaggcag

SEQ ID NO: 43 - DNA, 20 bp, Homo sapiens
ctgcctggtaagtgtctatt

SEQ ID NO: 44 - RNA, 20 nt, Homo sapiens
auauaaucaacuccucugcc

SEQ ID NO: 45 - RNA, 20 nt, Homo sapiens
aauagacacuuaccaggcag

SEQ ID NO: 46 - DNA, 1428 bp, Homo sapiens
ggaagaacagcctttcaaatttgacttctgcatgtggatagatttctttacatattcatcaatgtctgaagaagttacttatgcagatcttcaattccagaactccagtgagatggaaaaaatcccagaaattggcaaatttggggaaaaagcacctccagctccctctcatgtatggcgtccagcagccttgtttctgactcttctgtgccttctgttgctcattggattgggagtcttggcaagcatgtttcacgtaactttgaagatagaaatgaaaaaaatgaacaaactacaaaacatcagtgaagagctccagagaaatatttctctacaactgatgagtaacatgaatatctccaacaagatcaggaacctctccaccacactgcaaacaatagccaccaaattatgtcgtgagctatatagcaaagaacaagagcacaaatgtaagccttgtccaaggagatggatttggcataaggacagctgttatttcctaagtgatgatgtccaaacatggcaggagagtaaaatggcctgtgctgctcagaatgccagcctgttgaagataaacaacaaaaatgcattggaatttataaaatcccagagtagatcatatgactattggctgggattatctcctgaagaagattccactcgtggtatgagagtggataatataatcaactcctctgcctgggttataagaaacgcacctgacttaaataacatgtattgtggatatataaatagactatatgttcaatattatcactgcacttataaaaaaagaatgatatgtgagaagatggccaatccagtgcagcttggttctacatattttagggaggcatgaggcatcaatcaaatacatttaaggagtgtagggggtgggggttctaggctataggtaaatttaaatattttctggttgacaattagttgagtttgtctgaagacctgggattttatcatgcagatgaaacatccaggtagcaagcttcagagagaatagactgtgaatgttaatgccagagaggtataatgaagcatgtcccacctcccactttccatcatggcctgaaccctggaggaagaggaagtccattcagatagttgtggggggccttcgaattttcattttcatttacgttcttccccttctggccaagatttgccagaggcaacatcaaaaaccagcaaattttaattttgtcccacagcgttgctagggtggcatggctccccatctcgggtccatcctatacttccatgggactccctatggctgaaggccttatgagtcaaaggacttatagccaattgattgttctaggccaggtaagaatggatatggacatgcatttattacctcttaaaattattattttaagtaaaagccaataaacaaaaacgaaaaggcaa

SEQ ID NO: 47-62 - skipped (16 entries)

SEQ ID NO: 63 - protein, 12 aa, Artificial Sequence
  source: /note=“Description of Artificial Sequence: Synthetic peptide”
GGGSGGGSGGGS

SEQ ID NO: 64 - DNA, 20 bp, Homo sapiens
ccccaggactactcactcct

SEQ ID NO: 65 - DNA, 20 bp, Homo sapiens
tttcttgagctgcagctggg

SEQ ID NO: 66 - DNA, 20 bp, Homo sapiens
agttcccacatcctggtgcg

SEQ ID NO: 67 - DNA, 20 bp, Homo sapiens
atgcagaagtcaaatttgaa

SEQ ID NO: 68 - DNA, 20 bp, Homo sapiens
tcaaatttgacttctgcatg

SEQ ID NO: 69 - DNA, 20 bp, Homo sapiens
tttgacttctgcatgtggat

SEQ ID NO: 70 - DNA, 20 bp, Homo sapiens
ggataggtgagtgtcttttg

SEQ ID NO: 71 - DNA, 20 bp, Homo sapiens
tttccatctcactggagttc

SEQ ID NO: 72 - DNA, 20 bp, Homo sapiens
tgggattttttccatctcac

SEQ ID NO: 73 - DNA, 20 bp, Homo sapiens
atggaaaaaatcccagaaat

SEQ ID NO: 74 - DNA, 20 bp, Homo sapiens
atcccagaaattggcaaatt

SEQ ID NO: 75 - DNA, 20 bp, Homo sapiens
tcccagaaattggcaaattt

SEQ ID NO: 76 - DNA, 20 bp, Homo sapiens
ccccaaatttgccaatttct

SEQ ID NO: 77 - DNA, 20 bp, Homo sapiens
tccccaaatttgccaatttc

SEQ ID NO: 78 - DNA, 20 bp, Homo sapiens
attggcaaatttggggaaaa

SEQ ID NO: 79 - DNA, 20 bp, Homo sapiens
aaaggtaagattttgagtta

SEQ ID NO: 80 - DNA, 20 bp, Homo sapiens
agagggagctggaggtgctg

SEQ ID NO: 81 - DNA, 20 bp, Homo sapiens
ctccagctccctctcatgta

SEQ ID NO: 82 - DNA, 20 bp, Homo sapiens
catacatgagagggagctgg

SEQ ID NO: 83 - DNA, 20 bp, Homo sapiens
cgccatacatgagagggagc

SEQ ID NO: 84 - DNA, 20 bp, Homo sapiens
gctggacgccatacatgaga

SEQ ID NO: 85 - DNA, 20 bp, Homo sapiens
tgctggacgccatacatgag

SEQ ID NO: 86 - DNA, 20 bp, Homo sapiens
agagtcagaaacaaggctgc

SEQ ID NO: 87 - DNA, 20 bp, Homo sapiens
gcacagaagagtcagaaaca

SEQ ID NO: 88 - DNA, 20 bp, Homo sapiens
ctgtgccttctgttgctcat

SEQ ID NO: 89 - DNA, 20 bp, Homo sapiens
ccttctgttgctcattggat

SEQ ID NO: 90 - DNA, 20 bp, Homo sapiens
cttctgttgctcattggatt

SEQ ID NO: 91 - DNA, 20 bp, Homo sapiens
ccaatccaatgagcaacaga

SEQ ID NO: 92 - DNA, 20 bp, Homo sapiens
gctcattggattgggagtct

SEQ ID NO: 93 - DNA, 20 bp, Homo sapiens
atatagctcacgacataatt

SEQ ID NO: 94 - DNA, 20 bp, Homo sapiens
aaggtaatcttgtattctct

SEQ ID NO: 95 - DNA, 20 bp, Homo sapiens
caaggcttacatttgtgctc

SEQ ID NO: 96 - DNA, 20 bp, Homo sapiens
acaaatgtaagccttgtcca

SEQ ID NO: 97 - DNA, 20 bp, Homo sapiens
gtaagccttgtccaaggaga

SEQ ID NO: 98 - DNA, 20 bp, Homo sapiens
cttgtccaaggagatggatt

SEQ ID NO: 99 - DNA, 20 bp, Homo sapiens
caaatccatctccttggaca

SEQ ID NO: 100 - DNA, 20 bp, Homo sapiens
aaggagatggatttggcata

SEQ ID NO: 101 - DNA, 20 bp, Homo sapiens
ttatgccaaatccatctcct

SEQ ID NO: 102 - DNA, 20 bp, Homo sapiens
taagtgatgatgtccaaaca

SEQ ID NO: 103 - DNA, 20 bp, Homo sapiens
atgtttggacatcatcactt

SEQ ID NO: 104 - DNA, 20 bp, Homo sapiens
tgatgatgtccaaacatggc

SEQ ID NO: 105 - DNA, 20 bp, Homo sapiens
aacatggcaggagagtaaaa

SEQ ID NO: 106 - DNA, 20 bp, Homo sapiens
ttttactctcctgccatgtt

SEQ ID NO: 107 - DNA, 20 bp, Homo sapiens
gctggcattctgagcagcac

SEQ ID NO: 108 - DNA, 20 bp, Homo sapiens
gttgtttatcttcaacaggc

SEQ ID NO: 109 - DNA, 20 bp, Homo sapiens
ttttgttgtttatcttcaac

SEQ ID NO: 110 - DNA, 20 bp, Homo sapiens
gataaacaacaaaaatgcat

SEQ ID NO: 111 - DNA, 20 bp, Homo sapiens
aaaatgcattggtaaagccc

SEQ ID NO: 112 - DNA, 20 bp, Homo sapiens
agactatctgtatttcctgt

SEQ ID NO: 113 - DNA, 20 bp, Homo sapiens
gggattttataaattcctac

SEQ ID NO: 114 - DNA, 20 bp, Homo sapiens
agagtagatcatatgactat

SEQ ID NO: 115 - DNA, 20 bp, Homo sapiens
atagtcatatgatctactct

SEQ ID NO: 116 - DNA, 20 bp, Homo sapiens
aatagtcatatgatctactc

SEQ ID NO: 117 - DNA, 20 bp, Homo sapiens
tagatcatatgactattggc

SEQ ID NO: 118 - DNA, 20 bp, Homo sapiens
agatcatatgactattggct

SEQ ID NO: 119 - DNA, 20 bp, Homo sapiens
cctgaagaagattccactcg

SEQ ID NO: 120 - DNA, 20 bp, Homo sapiens
ccacgagtggaatcttcttc

SEQ ID NO: 121 - DNA, 20 bp, Homo sapiens
ttccactcgtggtatgagag

SEQ ID NO: 122 - DNA, 20 bp, Homo sapiens
atccactctcataccacgag

SEQ ID NO: 123 - DNA, 20 bp, Homo sapiens
aacaagaatagacacttacc

SEQ ID NO: 124 - DNA, 20 bp, Homo sapiens
aattctttactttctctttc

SEQ ID NO: 125 - DNA, 20 bp, Homo sapiens
attctttactttctctttca

SEQ ID NO: 126 - DNA, 20 bp, Homo sapiens
gacttaaataacatgtattg

SEQ ID NO: 127 - DNA, 20 bp, Homo sapiens
caatacatgttatttaagtc

SEQ ID NO: 128 - DNA, 20 bp, Homo sapiens
aagaatgatatgtgagaaga

SEQ ID NO: 129 - DNA, 20 bp, Homo sapiens
atggccaatccagtgcagct

SEQ ID NO: 130 - DNA, 20 bp, Homo sapiens
agaaccaagctgcactggat

SEQ ID NO: 131 - DNA, 20 bp, Homo sapiens
tatgtagaaccaagctgcac

SEQ ID NO: 132 - DNA, 20 bp, Homo sapiens
agcttggttctacatatttt

SEQ ID NO: 133 - DNA, 20 bp, Homo sapiens
gcttggttctacatatttta

SEQ ID NO: 134 - DNA, 20 bp, Homo sapiens
tggttctacatattttaggg

SEQ ID NO: 135 - DNA, 20 bp, Homo sapiens
acatattttagggaggcatg

SEQ ID NO: 136 - DNA, 20 bp, Homo sapiens
tattttgaagatcagagatg

SEQ ID NO: 137 - DNA, 20 bp, Homo sapiens
atcttcaaaatatcatgatt

SEQ ID NO: 138 - DNA, 20 bp, Homo sapiens
tcttcaaaatatcatgattt

SEQ ID NO: 139 - DNA, 20 bp, Homo sapiens
tatcatgatttgggttttcc

SEQ ID NO: 140 - DNA, 20 bp, Homo sapiens
tctcttcttgttttacttcc

SEQ ID NO: 141 - DNA, 20 bp, Homo sapiens
ctgtccacctctttgcatta

SEQ ID NO: 142 - DNA, 20 bp, Homo sapiens
cttaatgcaaagaggtggac

SEQ ID NO: 143 - DNA, 20 bp, Homo sapiens
cctctttgcattaaggtagt

SEQ ID NO: 144 - DNA, 20 bp, Homo sapiens
actaccttaatgcaaagagg

SEQ ID NO: 145 - DNA, 20 bp, Homo sapiens
ccaactaccttaatgcaaag

SEQ ID NO: 146 - DNA, 20 bp, Homo sapiens
ttaaggtagttggtccacct

SEQ ID NO: 147 - DNA, 20 bp, Homo sapiens
gaaggctggcatgaccaagg

SEQ ID NO: 148 - DNA, 20 bp, Homo sapiens
gaggaaggctggcatgacca

SEQ ID NO: 149 - DNA, 20 bp, Homo sapiens
ttaatgtgtgagaggaaggc

SEQ ID NO: 150 - DNA, 20 bp, Homo sapiens
ttatttaatgtgtgagagga

SEQ ID NO: 151 - DNA, 20 bp, Homo sapiens
aacattatttaatgtgtgag

SEQ ID NO: 152 - DNA, 20 bp, Homo sapiens
ataatgtttgttctcacaga

SEQ ID NO: 153 - DNA, 20 bp, Homo sapiens
tgtttgttctcacagaaggt

SEQ ID NO: 154 - DNA, 20 bp, Homo sapiens
gtttgttctcacagaaggta

SEQ ID NO: 155 - DNA, 20 bp, Homo sapiens
attgtgcttattttcataat

SEQ ID NO: 156 - DNA, 20 bp, Homo sapiens
aattgtgcttattttcataa

SEQ ID NO: 157 - DNA, 20 bp, Homo sapiens
gagtttttggaagatggaaa

SEQ ID NO: 158 - DNA, 20 bp, Homo sapiens
aggtttgagtttttggaaga

SEQ ID NO: 159 - DNA, 20 bp, Homo sapiens
ccaaaaactcaaacctgtga

SEQ ID NO: 160 - DNA, 20 bp, Homo sapiens
caaaaactcaaacctgtgaa

SEQ ID NO: 161 - DNA, 20 bp, Homo sapiens
ccttcacaggtttgagtttt

SEQ ID NO: 162 - DNA, 20 bp, Homo sapiens
tattctaactttcccttcac

SEQ ID NO: 163 - DNA, 20 bp, Homo sapiens
tcagaaaatatgtaagtcac

SEQ ID NO: 164 - DNA, 20 bp, Homo sapiens
ctggaagtgggaggatggct

SEQ ID NO: 165 - DNA, 20 bp, Homo sapiens
gaggtctggaagtgggagga

SEQ ID NO: 166 - DNA, 20 bp, Homo sapiens
gaatgaggtctggaagtggg

SEQ ID NO: 167 - DNA, 20 bp, Homo sapiens
gtcgaatgaggtctggaagt

SEQ ID NO: 168 - DNA, 20 bp, Homo sapiens
agtcgaatgaggtctggaag

SEQ ID NO: 169 - DNA, 20 bp, Homo sapiens
tccagacctcattcgacttc

SEQ ID NO: 170 - DNA, 20 bp, Homo sapiens
gccagaagtcgaatgaggtc

SEQ ID NO: 171 - DNA, 20 bp, Homo sapiens
atctcgccagaagtcgaatg

SEQ ID NO: 172 - DNA, 20 bp, Homo sapiens
actcatttaattcgttgggt

SEQ ID NO: 173 - DNA, 20 bp, Homo sapiens
ccaacgaattaaatgagtcc

SEQ ID NO: 174 - DNA, 20 bp, Homo sapiens
caggactcatttaattcgtt

SEQ ID NO: 175 - DNA, 20 bp, Homo sapiens
ccaggactcatttaattcgt

SEQ ID NO: 176 - DNA, 20 bp, Homo sapiens
aataggagtacatcaaagcc

SEQ ID NO: 177 - DNA, 20 bp, Homo sapiens
tgtttatttatgtttacaat

SEQ ID NO: 178 - DNA, 23 bp, Homo sapiens
acctgtcaggtgaagttcgctgg

SEQ ID NO: 179 - DNA, 23 bp, Homo sapiens
tggccgggttctagagtgccagg

SEQ ID NO: 180 - DNA, 23 bp, Homo sapiens
ggccgggttctagagtgccaggg

SEQ ID NO: 181 - DNA, 23 bp, Homo sapiens
caccgaggagtgagtagtcctgg

SEQ ID NO: 182 - DNA, 23 bp, Homo sapiens
tccagcgaacttcacctgacagg

SEQ ID NO: 183 - DNA, 20 bp, Homo sapiens
gctgtggggagaggggttgt

SEQ ID NO: 184 - DNA, 20 bp, Homo sapiens
ctgtggggagaggggttgtc

SEQ ID NO: 185 - DNA, 20 bp, Homo sapiens
tggggaaacgagggtcagct

SEQ ID NO: 186 - DNA, 20 bp, Homo sapiens
gggcccctgtggggaaacga

SEQ ID NO: 187 - DNA, 20 bp, Homo sapiens
agggcccctgtggggaaacg

SEQ ID NO: 188 - DNA, 20 bp, Homo sapiens
gctgaccctcgtttccccac

SEQ ID NO: 189 - DNA, 20 bp, Homo sapiens
ctgaccctcgtttccccaca

SEQ ID NO: 190 - DNA, 20 bp, Homo sapiens
tgaccctcgtttccccacag

SEQ ID NO: 191 - DNA, 20 bp, Homo sapiens
ccatagccagggcccctgtg

SEQ ID NO: 192 - DNA, 20 bp, Homo sapiens
gcatgtgacaggtgaggcac

SEQ ID NO: 193 - DNA, 20 bp, Homo sapiens
tgaggcacaggcttcagaag

SEQ ID NO: 194 - DNA, 20 bp, Homo sapiens
aggcttcagaagtggccgca

SEQ ID NO: 195 - DNA, 20 bp, Homo sapiens
ggcttcagaagtggccgcaa

SEQ ID NO: 196 - DNA, 20 bp, Homo sapiens
gtacccatgaacttcccttg

SEQ ID NO: 197 - DNA, 20 bp, Homo sapiens
gtggccgcaagggaagttca

SEQ ID NO: 198 - DNA, 20 bp, Homo sapiens
tggccgcaagggaagttcat

SEQ ID NO: 199 - DNA, 20 bp, Homo sapiens
ggaagttcatgggtactgca

SEQ ID NO: 200 - DNA, 20 bp, Homo sapiens
ttcatgggtactgcagggca

SEQ ID NO: 201 - DNA, 20 bp, Homo sapiens
ctaaacccctcccagtacca

SEQ ID NO: 202 - DNA, 20 bp, Homo sapiens
cactcacctgcccacagcag

SEQ ID NO: 203 - DNA, 20 bp, Homo sapiens
ccctgctgtgggcaggtgag

SEQ ID NO: 204 - DNA, 20 bp, Homo sapiens
tgggcaggtgagtggctgtg

SEQ ID NO: 205 - DNA, 20 bp, Homo sapiens
ggtgagtggctgtggggaga

SEQ ID NO: 206 - DNA, 20 bp, Homo sapiens
gtgagtggctgtggggagag

SEQ ID NO: 207 - DNA, 20 bp, Homo sapiens
atccatagccagggcccctg

SEQ ID NO: 208 - DNA, 20 bp, Homo sapiens
tccatagccagggcccctgt

SEQ ID NO: 209 - DNA, 20 bp, Homo sapiens
tcgtttccccacaggggccc

SEQ ID NO: 210 - DNA, 20 bp, Homo sapiens
tggctatggatccaaatttc

SEQ ID NO: 211 - DNA, 23 bp, Homo sapiens
agaaatttggatccatagccagg

SEQ ID NO: 212 - DNA, 23 bp, Homo sapiens
atccctggcactctagaacccgg

SEQ ID NO: 213 - DNA, 23 bp, Homo sapiens
cctcactagacttgacccacagg

SEQ ID NO: 214 - protein, 41 aa, Artificial Sequence
  source: /note=“Description of Artificial Sequence: Synthetic polypeptide”
KRGRKKLLYIFKQPFMRPVQTTQEEDGCSCRFPEEEEGGCE

SEQ ID NO: 215 - protein, 69 aa, Artificial Sequence
  source: /note=“Description of Artificial Sequence: Synthetic polypeptide”
TTTPAPRPPTPAPTIASQPLSLRPEACRPAAGGAVHTRGLDFACDIYIWAPLAGTCGVLLLSLVITLYC

SEQ ID NO: 216 - RNA, 20 nt, Homo sapiens
auggaaaaaaucccagaaau

SEQ ID NO: 217 - RNA, 20 nt, Homo sapiens
aucccagaaauuggcaaauu

SEQ ID NO: 218 - RNA, 20 nt, Homo sapiens
ucccagaaauuggcaaauuu

SEQ ID NO: 219 - RNA, 20 nt, Homo sapiens
auuggcaaauuuggggaaaa

SEQ ID NO: 220 - RNA, 20 nt, Homo sapiens
uccccaaauuugccaauuuc

SEQ ID NO: 221 - RNA, 20 nt, Homo sapiens
ugggauuuuuuccaucucac

SEQ ID NO: 222 - RNA, 20 nt, Homo sapiens
uuuccaucucacuggaguuc

SEQ ID NO: 223 - RNA, 20 nt, Homo sapiens
cuccagcucccucucaugua

SEQ ID NO: 224 - RNA, 20 nt, Homo sapiens
cugugccuucuguugcucau

SEQ ID NO: 225 - RNA, 20 nt, Homo sapiens
ccuucuguugcucauuggau

SEQ ID NO: 226 - RNA, 20 nt, Homo sapiens
cuucuguugcucauuggauu

SEQ ID NO: 227 - RNA, 20 nt, Homo sapiens
gcucauuggauugggagucu

SEQ ID NO: 228 - RNA, 20 nt, Homo sapiens
ccaauccaaugagcaacaga

SEQ ID NO: 229 - RNA, 20 nt, Homo sapiens
gcacagaagagucagaaaca

SEQ ID NO: 230 - RNA, 20 nt, Homo sapiens
agagucagaaacaaggcugc

SEQ ID NO: 231 - RNA, 20 nt, Homo sapiens
ugcuggacgccauacaugag

SEQ ID NO: 232 - RNA, 20 nt, Homo sapiens
gcuggacgccauacaugaga

SEQ ID NO: 233 - RNA, 20 nt, Homo sapiens
cgccauacaugagagggagc

SEQ ID NO: 234 - RNA, 20 nt, Homo sapiens
cauacaugagagggagcugg

SEQ ID NO: 235 - RNA, 20 nt, Homo sapiens
agagggagcuggaggugcug

SEQ ID NO: 236 - RNA, 20 nt, Homo sapiens
auauagcucacgacauaauu

SEQ ID NO: 237 - RNA, 20 nt, Homo sapiens
acaaauguaagccuugucca

SEQ ID NO: 238 - RNA, 20 nt, Homo sapiens
guaagccuuguccaaggaga

SEQ ID NO: 239 - RNA, 20 nt, Homo sapiens
cuuguccaaggagauggauu

SEQ ID NO: 240 - RNA, 20 nt, Homo sapiens
aaggagauggauuuggcaua

SEQ ID NO: 241 - RNA, 20 nt, Homo sapiens
uaagugaugauguccaaaca

SEQ ID NO: 242 - RNA, 20 nt, Homo sapiens
ugaugauguccaaacauggc

SEQ ID NO: 243 - RNA, 20 nt, Homo sapiens
aacauggcaggagaguaaaa

SEQ ID NO: 244 - RNA, 20 nt, Homo sapiens
gauaaacaacaaaaaugcau

SEQ ID NO: 245 - RNA, 20 nt, Homo sapiens
aaaaugcauugguaaagccc

SEQ ID NO: 246 - RNA, 20 nt, Homo sapiens
uuuuguuguuuaucuucaac

SEQ ID NO: 247 - RNA, 20 nt, Homo sapiens
guuguuuaucuucaacaggc

SEQ ID NO: 248 - RNA, 20 nt, Homo sapiens
gcuggcauucugagcagcac

SEQ ID NO: 249 - RNA, 20 nt, Homo sapiens
uuuuacucuccugccauguu

SEQ ID NO: 250 - RNA, 20 nt, Homo sapiens
auguuuggacaucaucacuu

SEQ ID NO: 251 - RNA, 20 nt, Homo sapiens
uuaugccaaauccaucuccu

SEQ ID NO: 252 - RNA, 20 nt, Homo sapiens
caaauccaucuccuuggaca

SEQ ID NO: 253 - RNA, 20 nt, Homo sapiens
caaggcuuacauuugugcuc

SEQ ID NO: 254 - RNA, 20 nt, Homo sapiens
agaguagaucauaugacuau

SEQ ID NO: 255 - RNA, 20 nt, Homo sapiens
uagaucauaugacuauuggc

SEQ ID NO: 256 - RNA, 20 nt, Homo sapiens
agaucauaugacuauuggcu

SEQ ID NO: 257 - RNA, 20 nt, Homo sapiens
ccugaagaagauuccacucg

SEQ ID NO: 258 - RNA, 20 nt, Homo sapiens
uuccacucgugguaugagag

SEQ ID NO: 259 - RNA, 20 nt, Homo sapiens
auccacucucauaccacgag

SEQ ID NO: 260 - RNA, 20 nt, Homo sapiens
ccacgaguggaaucuucuuc

SEQ ID NO: 261 - RNA, 20 nt, Homo sapiens
aauagucauaugaucuacuc

SEQ ID NO: 262 - RNA, 20 nt, Homo sapiens
auagucauaugaucuacucu

SEQ ID NO: 263 - RNA, 20 nt, Homo sapiens
gggauuuuauaaauuccuac

SEQ ID NO: 264 - RNA, 20 nt, Homo sapiens
gacuuaaauaacauguauug

SEQ ID NO: 265 - RNA, 20 nt, Homo sapiens
aagaaugauaugugagaaga

SEQ ID NO: 266 - RNA, 20 nt, Homo sapiens
auggccaauccagugcagcu

SEQ ID NO: 267 - RNA, 20 nt, Homo sapiens
uauguagaaccaagcugcac

SEQ ID NO: 268 - RNA, 20 nt, Homo sapiens
agaaccaagcugcacuggau

SEQ ID NO: 269 - RNA, 20 nt, Homo sapiens
caauacauguuauuuaaguc

SEQ ID NO: 270 - DNA, 1476 bp, Homo sapiens
ctatttagcattgctgctgccagccccaaccacatttctgattgcctaggaagaacagcctttcaaatttgacttctgcatgtggatagatttctttacatattcatcaatgtctgaagaagttacttatgcagatcttcaattccagaactccagtgagatggaaaaaatcccagaaattggcaaatttggggaaaaagcacctccagctccctctcatgtatggcgtccagcagccttgtttctgactcttctgtgccttctgttgctcattggattgggagtcttggcaagcatgtttcacgtaactttgaagatagaaatgaaaaaaatgaacaaactacaaaacatcagtgaagagctccagagaaatatttctctacaactgatgagtaacatgaatatctccaacaagatcaggaacctctccaccacactgcaaacaatagccaccaaattatgtcgtgagctatatagcaaagaacaagagcacaaatgtaagccttgtccaaggagatggatttggcataaggacagctgttatttcctaagtgatgatgtccaaacatggcaggagagtaaaatggcctgtgctgctcagaatgccagcctgttgaagataaacaacaaaaatgcattggaatttataaaatcccagagtagatcatatgactattggctgggattatctcctgaagaagattccactcgtggtatgagagtggataatataatcaactcctctgcctgggttataagaaacgcacctgacttaaataacatgtattgtggatatataaatagactatatgttcaatattatcactgcacttataaaaaaagaatgatatgtgagaagatggccaatccagtgcagcttggttctacatattttagggaggcatgaggcatcaatcaaatacatttaaggagtgtagggggtgggggttctaggctataggtaaatttaaatattttctggttgacaattagttgagtttgtctgaagacctgggattttatcatgcagatgaaacatccaggtagcaagcttcagagagaatagactgtgaatgttaatgccagagaggtataatgaagcatgtcccacctcccactttccatcatggcctgaaccctggaggaagaggaagtccattcagatagttgtggggggccttcgaattttcattttcatttacgttcttccccttctggccaagatttgccagaggcaacatcaaaaaccagcaaattttaattttgtcccacagcgttgctagggtggcatggctccccatctcgggtccatcctatacttccatgggactccctatggctgaaggccttatgagtcaaaggacttatagccaattgattgttctaggccaggtaagaatggatatggacatgcatttattacctcttaaaattattattttaagtaaaagccaataaacaaaaacgaaaaggcaa

SEQ ID NO: 271 - DNA, 1553 bp, Homo sapiens
ggctcatttgcagacatatgggtgattggtacagtaggtttataaacagaagtttaaacttgtaagcttaagcttccgtttataaacagaagtttaaaattataggtcctgtttaacattcagctctgttaactcactcatctttttgtgtttttacactttgtcaagatttctttacatattcatcaatgtctgaagaagttacttatgcagatcttcaattccagaactccagtgagatggaaaaaatcccagaaattggcaaatttggggaaaaagcacctccagctccctctcatgtatggcgtccagcagccttgtttctgactcttctgtgccttctgttgctcattggattgggagtcttggcaagcatgtttcacgtaactttgaagatagaaatgaaaaaaatgaacaaactacaaaacatcagtgaagagctccagagaaatatttctctacaactgatgagtaacatgaatatctccaacaagatcaggaacctctccaccacactgcaaacaatagccaccaaattatgtcgtgagctatatagcaaagaacaagagcacaaatgtaagccttgtccaaggagatggatttggcataaggacagctgttatttcctaagtgatgatgtccaaacatggcaggagagtaaaatggcctgtgctgctcagaatgccagcctgttgaagataaacaacaaaaatgcattggaatttataaaatcccagagtagatcatatgactattggctgggattatctcctgaagaagattccactcgtggtatgagagtggataatataatcaactcctctgcctgaaaatatcaaacgaagaaagaaaccagagtctcaacctgctggacactattggaagtccatcatttaacacgtttttagtatatacttttagcaggagacagctctgagtcaactgtgttgaggtgccaccacagcgagtttaggcactcagatccctgcatactcatcacattgggccataatggcaaatagaattttttgttttgttttgtttgtttgctttttctttcacatagaaatagtaagtgtaggagtgtgggtcagaaagaaaaggtggccctacctctgatggttggcaatgataggatacaatgggagataagctatctacaaatggagtggagaaggatatatatttcaaaggcctaatttgtagtgaaagactagagacaaaggtaatgtgtgtgtcaggagagagtacagatggaatcttgttttgcaaacgtagaatatgtatgtgtttgtaattattgcaaatggaatggtaatctataatggaatggaaaacattgtagatattttcagttatcaaaaagaaaactgaaaaagtatataataattgtatgtatgatatatatatgtgtgtgtgtgtgtatatatatcttcactttataactctgtgttgttttggggtttgtttctgaaagggggttgtaataaatgacatctgtactatgtcaccacaaataaatctcattcttaaacatttaattgatgaactta

SEQ ID NO: 272 - DNA, 1456 bp, Homo sapiens
ggctcatttgcagacatatgggtgattggtacagtaggtttataaacagaagtttaaacttgtaagcttaagcttccgtttataaacagaagtttaaaattataggtcctgtttaacattcagctctgttaactcactcatctttttgtgtttttacactttgtcaagatttctttacatattcatcaatgtctgaagaagttacttatgcagatcttcaattccagaactccagtgagatggaaaaaatcccagaaattggcaaatttggggaaaaagttcacgtaactttgaagatagaaatgaaaaaaatgaacaaactacaaaacatcagtgaagagctccagagaaatatttctctacaactgatgagtaacatgaatatctccaacaagatcaggaacctctccaccacactgcaaacaatagccaccaaattatgtcgtgagctatatagcaaagaacaagagcacaaatgtaagccttgtccaaggagatggatttggcataaggacagctgttatttcctaagtgatgatgtccaaacatggcaggagagtaaaatggcctgtgctgctcagaatgccagcctgttgaagataaacaacaaaaatgcattggaatttataaaatcccagagtagatcatatgactattggctgggattatctcctgaagaagattccactcgtggtatgagagtggataatataatcaactcctctgcctgggttataagaaacgcacctgacttaaataacatgtattgtggatatataaatagactatatgttcaatattatcactgcacttataaaaaaagaatgatatgtgagaagatggccaatccagtgcagcttggttctacatattttagggaggcatgaggcatcaatcaaatacatttaaggagtgtagggggtgggggttctaggctataggtaaatttaaatattttctggttgacaattagttgagtttgtctgaagacctgggattttatcatgcagatgaaacatccaggtagcaagcttcagagagaatagactgtgaatgttaatgccagagaggtataatgaagcatgtcccacctcccactttccatcatggcctgaaccctggaggaagaggaagtccattcagatagttgtggggggccttcgaattttcattttcatttacgttcttccccttctggccaagatttgccagaggcaacatcaaaaaccagcaaattttaattttgtcccacagcgttgctagggtggcatggctccccatctcgggtccatcctatacttccatgggactccctatggctgaaggccttatgagtcaaaggacttatagccaattgattgttctaggccaggtaagaatggatatggacatgcatttattacctcttaaaattattattttaagtaaaagccaataaacaaaaacgaaaaggcaa